(19) United States
(12) Patent Application Publication
(10) Pub. No.: US 2021/0191606 A1
(43) Pub. Date: Jun. 24, 2021

(54) HEALTHCARE CONTENT MANAGEMENT SYSTEM

(71) Applicant: RICOH COMPANY, LTD., Tokyo (JP)

(72) Inventors: Jayasimha Nuggehalli, Sunnyvale, CA (US); James Woo, Los Altos, CA (US); Bhushan Nadkarni, Santa Clara, CA (US)

(21) Appl. No.: 17/129,336

(22) Filed: Dec. 21, 2020

Related U.S. Application Data
(63) Continuation of application No. 14/937,769, filed on Nov. 10, 2015.

(51) Int. Cl. G06F 3/0484; G16H 30/20

(52) U.S. Cl. CPC G06F 3/04845; G16H 30/20; G06F 3/04842

(57) ABSTRACT

An image and metadata for the image are received. The metadata includes image identification data. A first graphical user interface is generated and displayed for a user. The image is displayed in a first portion of the first graphical user interface. The metadata is displayed in a second portion of the first graphical user interface. One or more first interactive elements for processing the image and the metadata are displayed in a third portion of the first graphical user interface. Verification of whether the image is to be associated with the metadata is performed by: determining whether first input indicating a first request to associate the image with the metadata is received via one or more first interactive elements; and in response to determining that the first input is received: associating the image with the metadata; and transmitting the image in association with the metadata to a storage device.

### BENEFIT CLAIM

This application claims the benefit under 35 U.S.C. § 120 as a Continuation of application Ser. No. 14/937,769, filed Nov. 10, 2015, the entire contents of which is hereby incorporated by reference for all purposes as if fully set forth herein. The applicants hereby rescind any disclaimer of claim scope in the parent applications or the prosecution history thereof and advise the USPTO that the claims in this application may be broader than any claim in the parent applications.

### RELATED APPLICATION DATA AND CLAIM OF PRIORITY

This application is related to U.S. patent application Ser. No. 14/543,712 (Attorney Docket No. 49986-0811) titled IMAGE ACQUISITION AND MANAGEMENT, filed Nov. 17, 2014, U.S. patent application Ser. No. 14/543,725 (Attorney Docket No. 49986-0817) titled IMAGE ACQUISITION AND MANAGEMENT, filed Nov. 17, 2014, U.S. patent application Ser. No. 14/619,533 (Attorney Docket No. 49986-0821) titled MANAGING ACCESS TO IMAGES USING ROLES, filed Feb. 11, 2015, U.S. patent application Ser. No. 14/619,550 (Attorney Docket 49986-0822) titled MANAGING ACCESS TO WORKFLOWS USING ROLES, filed Feb. 11, 2015, and U.S. patent application Ser. No. 14/937,765 (Attorney Docket 49986-0847) titled HEALTHCARE CONTENT MANAGEMENT SYSTEM, filed Nov. 10, 2015, the contents all of which are incorporated by reference in their entirety for all purposes as if fully set forth herein.

### FIELD OF THE INVENTION

Embodiments relate generally to managing the process of assigning metadata to images and images to patient records in healthcare-related-applications.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Applicability of devices capable of capturing images for medical purposes is constantly increasing. These days, the images may be captured by cameras installed in mobile devices, such as smartphones and tablet computers, as well as by scanners installed in various mobile and stationary devices. However, the flexibility of receiving the images from different devices may cause difficulties in processing such images. One of the issues related to the processing of the images captured or otherwise provided by a plurality of devices is that it is often difficult to catalogue the images or assign them to the corresponding patient records.

The processing may be especially difficult if there is a vast amount of images and if there are many ways of providing image identifications for the images. For example, the received images may be represented in different formats. Some of the images may depict information about the image identification, while other images may be identified by referring to additional files or hyperlinks. The heterogenic nature of the images and the contents the images may make the process of assigning metadata to images and the images to the patient's record especially difficult.

### SUMMARY

According to an embodiment, a network device includes one or more processors, one or more memories, and an image management application configured to receive an image acquired by a device. Metadata for an image is also received.

In an embodiment, an image management application is configured to generate and display a first graphical user interface for displaying the image and the metadata. The image may be displayed in a first portion of the first graphical user interface and the metadata may be displayed in a second portion of the first graphical user interface. One or more first interactive elements for processing the image and the metadata may be displayed in a third portion of the first graphical user interface.

In an embodiment, an image management application verifies whether the image is to be associated with the metadata by determining whether first input indicating a first request to associate the image with the metadata is received via one or more first interactive elements. In response to determining that the first input indicating the first request to associate the image with the metadata is received, the image is associated with the metadata, and the image in association with the metadata is transmitted to a storage device.

### BRIEF DESCRIPTION OF THE DRAWINGS

In the figures of the accompanying drawings like reference numerals refer to similar elements.

FIG. 1 is a block diagram that depicts an arrangement for acquiring and managing images.

FIG. 2 is a flow diagram that depicts an approach for a mobile device to acquire images using a reference image as a background image and a distance at which the reference image was acquired.

FIG. 3A depicts an example reference image that includes one or more objects that are represented by different shapes.

FIG. 3B depicts a distance at which a reference image was acquired.

FIG. 3C depicts a preview image displayed on a mobile device display.

FIG. 3D depicts a mobile device that has been positioned and oriented so that the one or more objects in a reference image and one or more preview images overlap

FIG. 4A depicts top-level information that includes a patient identification field (“ID Scan”), an anatomy identification field (“Anatomy ID”), a department field (“Department”), a status field (“Status”) and a registered nurse name (“RN—Name”).

FIG. 4B depicts that a user has used one or more controls (graphical or physical) on a mobile device to navigate to the department field.

FIG. 4C depicts the department options available to the user after selecting the department field and that the user has navigated to the Dermatology department option.

FIG. 4D depicts a graphical user interface that allows the user to specify a wristband setting, a body part, a wound type and an indication of the seriousness of the injury.

FIG. 5A depicts a table of example types of memorandum data.

FIG. 5B is a table that depicts a textual representation of image data 552 that includes embedded audio data.

FIG. 6A depicts an example login screen that queries a user for user credentials that include a user login ID and password.

FIG. 6B depicts an example dashboard screen that provides access to various functionality for managing image data.

FIG. 6C depicts an example Approval Queue screen, or work queue, that allows a user to view and approve or reject images.

FIG. 6D depicts an example Rejected Image Processing screen that allows a user to view and update information for rejected images.

FIG. 7A is a table that depicts an example patient database, where each row of the table corresponds to a patient and specifies an identifier, a date of birth (DOB), a gender, an ID list, a social security number (SSN), a sending facility, a family name, a first (given) name and another given (middle) name.

FIG. 7B is a table that depicts an example patient database schema.

FIG. 8 depicts an example historical view screen generated by image management application.

FIG. 9 is a flow diagram that depicts an approach for managing access to images using logical entities.

FIG. 10 depicts a table of example types of memorandum data that may be included in the metadata for an image.

FIG. 11 depicts an example GUI screen after a user has been granted access to a requested image.

FIG. 12 depicts an example user table schema that defines an example data schema for users.

FIG. 13 depicts an example user table that specifies various types of user data.

FIG. 14 depicts an example GUI specifying user data.

FIG. 15 is a table that depicts four example levels of access to workflows and images.

FIG. 16A is a flow diagram that depicts an approach for managing access to a workflow using the access criteria for Level 1.

FIG. 16B is a flow diagram that depicts an approach for managing access to a workflow using the access criteria for Level 2.

FIG. 16C is a flow diagram that depicts an approach for managing access to a workflow using the access criteria for Level 3.

FIG. 16D is a flow diagram that depicts an approach for managing access to a workflow using the access criteria for Level 4.

FIG. 17 depicts an example user table that specifies various types of user data.

FIG. 18 depicts a table of example types of memorandum data that may be included in the metadata for an image.

FIG. 19 depicts an example workflow schema that defines an example data schema for workflows.

FIG. 20A depicts an example workflow for processing images.

FIG. 20B depicts an example workflow that includes all of the elements of the workflow of FIG. 20A, and also includes an additional Approval Queue at Level 3.

FIG. 20C depicts an example workflow that is the same as workflow of FIG. 20A, except that approved images are provided to storage instead of an EMR system.

FIG. 21 is a block diagram that depicts an example computer system upon which embodiments may be implemented.

FIG. 22A is a block diagram that depicts an arrangement for acquiring and managing digital images received from a multifunction peripheral device and transmitted to a file server.

FIG. 22B is a block diagram that depicts an arrangement for acquiring and managing digital images received from a multifunction peripheral device and stored in a data folder.

FIG. 22C is a block diagram that depicts an arrangement for acquiring and managing digital images received from a server and transmitted to a file server.

FIG. 22D is a block diagram that depicts an arrangement for acquiring and managing digital images received from a server and stored in a data folder.

FIG. 23 is an example digital image that includes metadata represented as barcodes and metadata represented as alphanumeric strings.

FIG. 24 is an example of a fax cover sheet containing metadata represented as alphanumerical strings.

FIG. 25 depicts an example graphical user interface that allows a user to review an image and metadata and determine whether the image is to be associated with a patient record.

FIG. 26 depicts an example graphical user interface that allows a user to review an image and metadata and determine whether the image is to be accepted or discarded.

FIG. 27 depicts an example graphical user interface that allows a user to search patient records to determine a patient record for an image.

FIG. 28 depicts an example graphical user interface that allows a user to validate an image if a patient record for the image has been verified.

FIG. 29 depicts an example graphical user interface that allows a user to augment metadata associated with the image.

FIG. 30 depicts an example workflow for a document integration process.

FIG. 31 depicts a block diagram that depicts an arrangement for capturing metadata for images using a desktop computer.

FIG. 32 depicts a block diagram that depicts an arrangement for capturing metadata for images using a portable device.

FIG. 33 depicts a block diagram that depicts an arrangement for transmitting images and metadata as electronic mail attachments.

FIG. 34 depicts a block diagram that depicts an arrangement for transmitting images and metadata to an electronic data folder.

FIG. 35 depicts an example workflow for a metadata assignment process.

FIG. 36 depicts an example data structure used to store metadata information.

FIG. 37 depicts an example interface for interactively assigning metadata to images.

### DETAILED DESCRIPTION

In the following description, for the purposes of explanation, numerous specific details are set forth in order to provide a thorough understanding of the embodiments. It will be apparent, however, to one skilled in the art that the embodiments may be practiced without these specific details. In other instances, well-known structures and devices are shown in block diagram form in order to avoid unnecessarily obscuring the embodiments.
I. OVERVIEW
II. SYSTEM ARCHITECTURE
   A. Mobile Device
   B. Application Server
III. ACQUIRING IMAGES USING A REFERENCE IMAGE AND DISTANCE
IV. MEMO AND AUDIO DATA
V. IMAGE DATA MANAGEMENT
VI. HISTORICAL VIEWS
VII. MANAGING ACCESS TO IMAGES USING ROLES
VIII. MANAGING ACCESS TO WORKFLOWS USING ROLES
   A. Access Levels
   B. Workflow Levels
IX. IMPLEMENTATION MECHANISMS
X. OVERVIEW OF A DOCUMENT INTERGRATION PROCESS
XI. WORKFLOW OF A DOCUMENT INTEGRATION PROCESS
XII. EXAMPLE DOCUMENT INTEGRATION PROCESS
XIII. ARRANGEMENTS FOR ACQUIRING AND MANAGING DIGITAL IMAGES
XIV. EXAMPLE ARRANGEMENTS FOR ACQUIRING AND MANAGING DIGITAL IMAGES RECEIVED FROM MULTIFUNCTION PERIPHERAL DEVICES
   A. File-Transfer-Based Arrangements
   B. Folder-Based Arrangements
XV. EXAMPLE ARRANGEMENTS FOR ACQUIRING AND MANAGING DIGITAL IMAGES RECEIVED FROM SERVERS
   A. File-transfer-based Arrangements
   B. Folder-based Arrangements
XVI. METADATA
   A. Example Metadata Represented as Barcodes and Alphanumerical Strings
   B. Example Metadata Represented as Alphanumerical Strings
XVII. VERIFICATION AND VALIDATION OF AN IMAGE INTEGRATION
   A. Association Validation
   B. Image Validation
   C. Patient Record Verification
   D. Image Validation When A Patient Record Has Been Verified
   E. Metadata Modification
XVIII. OVERVIEW OF A METADATA ASSIGNMENT PROCESS
XIX. WORKFLOW OF A METADATA ASSIGNMENT PROCESS
XX. EXAMPLE METADATA ASSIGNMENT PROCESS
XXI. ARRANGEMENTS FOR ASSIGNING METADATA TO IMAGES
   A. Example Arrangements For Providing Metadata For Images Using a Desktop Computer
   B. Example Arrangements For Assigning Metadata to Images Using a Portable Device
   C. Example Arrangements for Communicating Images and Metadata as Attachments
   D. Example Arrangements for Transmitting Images and Metadata to a Data Folder
XXII. EXAMPLE METADATA
XXIII. EXAMPLE INTERFACE FOR INTERACTIVE ASSIGNMENT

### I. OVERVIEW

An approach is provided for acquiring and managing images. According to the approach, a reference image of one or more objects is displayed on the display of a mobile device in a manner that allows a user of the mobile device to simultaneously view the reference image and a preview image of the one or more objects currently in a field of view of a camera of the mobile device. For example, the reference image may be displayed on the display of the mobile device at a different brightness level, color, or with special effects, relative to the preview image. An indication is provided to the user of the mobile device whether the camera of the mobile device is currently located within a specified amount of a distance at which the reference image was acquired. For example, a visual or audible indication may indicate whether the camera of the mobile device is too close, too far away, or within a specified amount of a distance at which the reference image was acquired. In response to a user request to acquire an image, the camera acquires a second image of the one or more objects and a distance between the camera and the one or more objects at the time the second image was acquired is recorded. The second image and metadata are transmitted to an image management application that is external to the mobile device. For example, the second image and metadata may be transmitted over one or more networks to the image management application executing on an application server. The image management application provides various functionalities for managing images. For example, the image management application may allow a user to review and accept images, reject images and update metadata for images. As another example, the image management application provides a historical view that allows a user to view a sequence of images of one or more objects that were acquired at approximately the same distance and angle, which allows a user to better discern changes over time in the one or more objects.

According to one embodiment, access to images, workflows and workflow levels is managed using roles. Users are assigned roles and users are permitted to access images, workflows and workflow levels for which they have been assigned the required roles.

### II. SYSTEM ARCHITECTURE

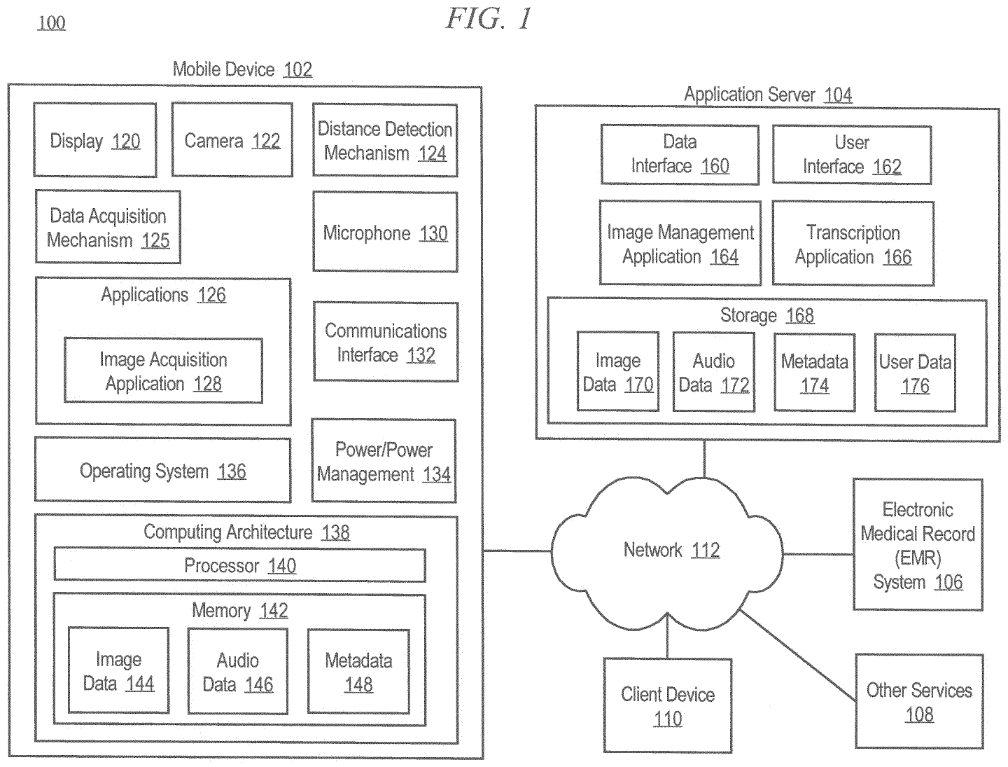

FIG. 1 is a block diagram that depicts an arrangement 100 for acquiring and managing images. Arrangement 100 includes a mobile device 102, an application server 104, an electronic medical record (EMR) system 106, other services 108 and a client device 110, communicatively coupled via a network 112. Arrangement 100 is not limited the particular elements depicted in FIG. 1 and may include fewer or additional elements depending upon a particular implementation. Embodiments are described herein in the context of a single mobile device 102 for purposes of explanation, but the approach is applicable to any number of mobile devices. Network 112 is depicted in FIG. 1 as a single network for purposes of explanation only and network 112 may include any number and type of wired or wireless networks, such as local area networks (LANs), wide area networks (WANs), the Internet, etc. The various elements depicted in FIG. 1 may also communicated with each other via direct communications links.

A. Mobile Device

Mobile device 102 may be any type of mobile device and examples of mobile device 102 include, without limitation, a smart phone, a camera, a tablet computing device, a personal digital assistant or a laptop computer. In the example depicted in FIG. 1, mobile device 102 includes a display 120, a camera 122, a distance detection mechanism 124, a data acquisition component 125, applications 126, including an image acquisition application 128, a microphone 130, a communications interface 132, a power/power management component 134, an operating system 136 and a computing architecture 138 that includes a processor 140 and memory 142, storing image data 144, audio data 146 and metadata 148. Mobile device 102 may include various other components that may vary depending upon a particular implementation and mobile device 102 is not limited to a particular set of components or features. For example, mobile device 102 may include a location component, such as one or more GPS components that is capable of determining a current location of mobile device 102 and generating location data that indicates the current location of mobile device 102. Mobile device 102 may also include manual controls, such as buttons, slides, etc., not depicted in FIG. 1, for performing various functions on mobile device, such as powering on/off or changing the state of mobile device 102 and/or display 120, or for acquiring digital images.

Display 120 may be implemented by any type of display that displays images and information to a user and may also be able to receive user input and embodiments are not limited to any particular implementation of display 120. Mobile device 102 may have any number of displays 120, of similar or varying types, located anywhere on mobile device 102. Camera 122 may be any type of camera and the type of camera may vary depending upon a particular implementation. As with display 120, mobile device 102 may be configured with any number of cameras 122 of similar or varying types, for example, on a front and rear surface of mobile device 102, but embodiments are not limited to any number or type of camera 122.

Distance detection mechanism 124 is configured to detect a distance between the camera 122 on mobile device 102 and one or more objects within the field of view of the camera 122. Example implementations of distance detection mechanism may be based upon, without limitation, infra-red, laser, radar, or other technologies that use electromagnetic radiation. Distance may be determined directly using the distance detection mechanism 124, or distance may be determined from image data. For example, the distance from the camera 122 to one or more objects on the ground and in the field of view of the camera 122 may be calculated based upon a height of the camera 122 and a current angle of the camera 122 with respect to the ground. For example, given a height (h) of the camera 122 and an acute angle (a) between the vertical and a line of sight to the one or more objects, the distance (d) may be calculated as follows: d=h*tan (a). As another example, if one or more dimensions of the one or more objects are known, the distance between the camera 122 and the one or more objects may be determined based upon a pixel analysis of the one or more objects for which the one or more dimensions are known.

Data acquisition component 125 may comprise hardware subcomponents, programmable subcomponents, or both. For example, data acquisition component 125 may include one or more cameras, scanners, memory units or other data storage units, buffers and code instructions for acquiring, storing and transmitting data, or any combination thereof. Data acquisition component 125 may be configured with a Wi-Fi interface and a barcode reader. The Wi-Fi interface may be used to transmit information to and from the data acquisition component 125. The barcode reader may be used to scan or otherwise acquire a code, such as a point of sale (POS) code displayed on an item.

Microphone 130 is configured to detect audio and in combination with other elements, may store audio data that represents audio detected by microphone 130. Communications interface 132 may include computer hardware, software, or any combination of computer hardware and software to provide wired and/or wireless communications links between mobile device 102 and other devices and/or networks. The particular components for communications interface 132 may vary depending upon a particular implementation and embodiments are not limited to any particular implementation of communications interface 132. Power/power management component 134 may include any number of components that provide and manage power for mobile device 102. For example, power/power management component 134 may include one or more batteries and supporting computer hardware and/or software to provide and manage power for mobile device 102.

Computing architecture 138 may include various elements that may vary depending upon a particular implementation and mobile device 102 is not limited to any particular computing architecture 138. In the example depicted in FIG. 1, computing architecture includes a processor 108 and a memory 142. Processor 108 may be any number and types of processors and memory 142 may be any number and types of memories, including volatile memory and non-volatile memory, which may vary depending upon a particular implementation. Computing architecture 138 may include additional hardware, firmware and software elements that may vary depending upon a particular implementation. In the example depicted in FIG. 1 memory 142 stores image data 144, audio data 146 and metadata 148, as described in more detail hereinafter, but memory 142 may store additional data depending upon a particular implementation.

Operating system 136 executes on computing architecture 138 and may be any type of operating system that may vary depending upon a particular implementation and embodiments are not limited to any particular implementation of operating system 136. Operating system 136 may include multiple operating systems of varying types, depending upon a particular implementation. Applications 126 may be any number and types of applications that execute on computing architecture 138 and operating system 136. Applications 126 may access components in mobile device 102, such as display 120, camera 122, distance detection mechanism 124, computing architecture 138, microphone 130, communications interface 132, power/power management component 134 and other components not depicted in FIG. 1, via one or more application program interfaces (APIs) for operating system 136.

Applications 126 may provide various functionalities that may vary depending upon a particular application and embodiments are not limited to applications 126 providing any particular functionality. Common non-limiting examples of applications 126 include social media applications, navigation applications, telephony, email and messaging applications, and Web service applications. In the example depicted in FIG. 1, applications 126 include an image acquisition application 128 that provides various functionalities for acquiring images. Example functionality includes allowing a user to acquire images via camera 122 while a reference image is displayed as a background image. In this example, the image acquisition application 128 is also configured to provide an indication to a user, e.g., a visual or audible indication, to indicate whether the camera 122 of the mobile device 102 is too close, too far away, or within a specified amount of a distance at which the reference image was acquired. Other example functionality includes acquiring metadata, memorandum data and/or audio data that corresponds to the acquired images, and transmitting this information with the acquired images to an image management application that is external to the mobile device 102. These and other example functionalities of image acquisition application 128 are described in more detail hereinafter. Image acquisition application 128 may be implemented in computer hardware, computer software, or any combination of computer hardware and software.

B. Application Server

In the example depicted in FIG. 1, application server 104 includes a data interface 160, a user interface 162, an image management application 164, a transcription application 166 and storage 168 that includes image data 170, audio data 172 and metadata 174. Application server 104 may include various other components that may vary depending upon a particular implementation and application server 104 is not limited to a particular set of components or features. Application server 104 may include various hardware and software components that may vary depending upon a particular implementation and application server 104 is not limited to any particular hardware and software components.

Data interface 160 is configured to receive data from mobile device 102 and may do so using various communication protocols and from various media. Example protocols include, without limitation, the File Transfer Protocol (FTP), the Telnet Protocol, the Transmission Control Protocol (TCP), the TCP/Internet Protocol (TCP/IP), the Hypertext Transfer Protocol (HTTP), the Simple Mail Transfer Protocol (SMTP), or any other data communications protocol. Data receiver 118 may be configured to read data from an FTP folder, an email folder, a Web server, a remote media such as a memory stick, or any other media. Data interface 160 may include corresponding elements to support these transport methods. For example, data interface 160 may include, or interact with, an FTP server that processes requests from an FTP client on mobile device 102. As another example, data interface 160 may include, or interact with, an email client for retrieving emails from an email server on mobile device 102 or external to mobile device 102. As yet another example, data interface 160 may include, or interact with, a Web server that responds to requests from an http client on mobile device 102. Data interface 160 is further configured to support the transmission of data from application server 104 to other devices and processes, for example, EMR system 106, other services 108 and client device 110.

User interface 160 provides a mechanism for a user, such as an administrator, to access application server 104 and data stored on storage 168, as described in more detail hereinafter. User interface 160 may be implemented as an API for application server 104. Alternatively, user interface 160 may be implemented by other mechanisms. For example, user interface 160 may be implemented as a Web server that serves Web pages to provide a user interface for application server 104.

Image management application 164 provides functionality for managing images received from mobile device 102 and stored in storage 168. Example functionality includes reviewing images, accepting images, rejecting images, processing images, for example to improve blurriness or otherwise enhance the quality of images, crop or rotate images, etc., as well as update metadata for images. Example functionality also includes providing a historical view of a sequence of images of one or more objects, where the images in the sequence were acquired using a reference image as a background image and at approximately the same distance from the one or more objects. According to one embodiment, image management application 164 provides a graphical user interface to allow user access to the aforementioned functionality. The graphical user interface may be provided by application software on client device 110, application software on application server 104, or any combination of application software on client device 110 and application server 104. As one example, the graphical user interface may be implemented by one or more Web pages generated on application server 104 and provided to client device 110. Image management application 164 may be implemented in computer hardware, computer software, or any combination of computer hardware and software. For example, image management application 164 may be implemented as an application, e.g., a Web application, executing on application server 104.

Transcription application 166 processes audio data acquired by mobile device 102 and generates a textual transcription. The textual transcription may be represented by data in any format that may vary depending upon a particular implementation. Storage 168 may include any type of storage, such as volatile memory and/or non-volatile memory. Application server 104 is configured to provide image and/or video data and identification data to EMR system 106, other services 108 and client device 110. Application server 104 transmits the data to EMR system 106, other services 108 and client device 110 using standard techniques or alternatively, Application server 104 may transmit data to EMR system 106, other services 108 and client device 110 in accordance with Application Program Interfaces (APIs) supported by EMR system 106, other services 108 and client device 110. Application server 104 may be implemented as a stand-alone network element, such as a server or intermediary device. Application server 104 may also be implemented on a client device, including mobile device 102.

### III. ACQUIRING IMAGES USING A REFERENCE IMAGE AND DISTANCE

According to one embodiment, mobile device 102 is configured to acquire image data using a reference image as a background image and a distance at which the reference image was acquired.

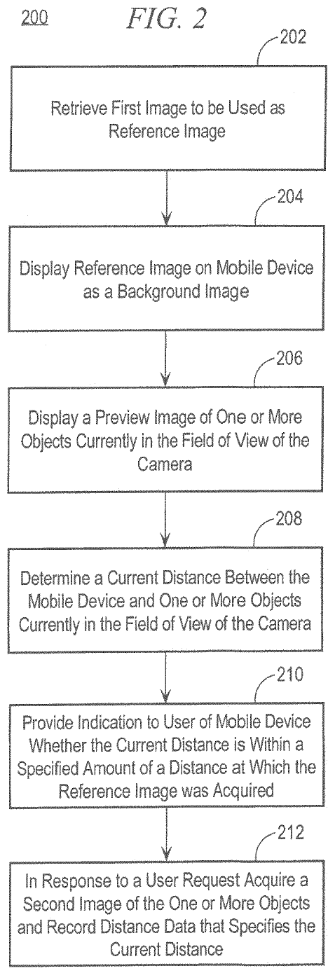

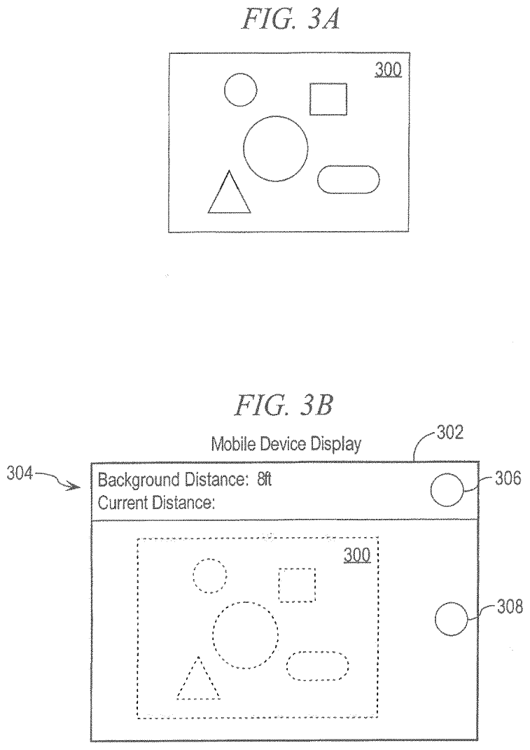

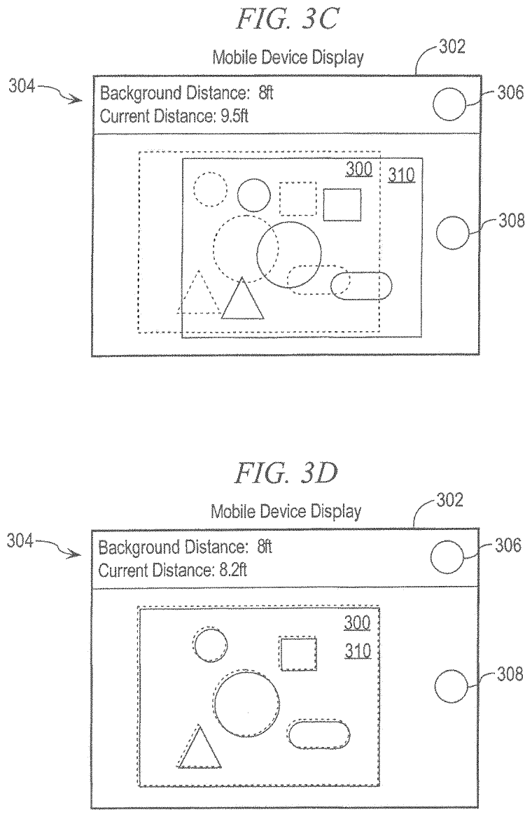

FIG. 2 is a flow diagram 200 that depicts an approach for a mobile device to acquire images using a reference image as a background image and a distance at which the reference image was acquired, according to an embodiment. In step 202, a reference image to be used as a reference image is retrieved. The reference image may be retrieved in response to a user invoking the image acquisition application 128 and specifying an image to be used as the reference image. For example, a user may select an icon on display 120 that corresponds to the image acquisition application 128 to invoke the image acquisition application 128 and the user is then queried for an image to be used as a reference image. The user may then select an image to be used as the reference image, or specify a location, e.g., a path, of an image to be used as the reference image. The reference image may originate and be retrieved from any source. For example, the reference image may have been acquired by mobile device 102 via camera 122 and be stored as image data 144 in memory 142, or at a location external to mobile device 102. As another example, the reference image may have been acquired by a device external to mobile device, such as client device 110, a scanner, or other services 108. The reference image data may be any type or format of image data. Example image data formats include, without limitation, raster formats such as JPEG, Exif, TIFF, RAW, GIF, BMP, PNG, PPM, PGM, PBM, PNM, etc., and vector formats such as CGM, SVG, etc. The reference image may have corresponding metadata 148 that describes one or more attributes of the reference image. Example attributes include, without limitation, camera settings used to acquire the reference image, and a distance from the camera used to acquire the reference image to the one or more objects in the reference image. FIG. 3A depicts an example reference image 300 that includes one or more objects that are represented by different shapes.

In step 204, the reference image is displayed on the mobile device as a background image. For example, image acquisition application 128 may cause the reference image to be displayed on display 120 of mobile device 102. FIG. 3B depicts an example mobile device display 302 that may be, for example, display 120 of mobile device 102. In this example, the reference image 300, which includes the one or more objects, is displayed on the mobile device display 302 as a background image in a manner that allows a user of the mobile device to simultaneously view a preview image of the one or more objects currently in a field of view of the camera. This may be accomplished using a wide variety of techniques that may vary depending upon a particular implementation and embodiments are not limited to any particular technique for displaying the reference image as a background image. For example, one or more attribute values for the reference image 300 may be changed. The attribute values may correspond to one or more attributes that affect the way in which the reference image appears on the mobile device display to a user. Example attributes include, without limitation, brightness, color or special effects. The reference image 300 may be displayed on mobile device display 302 using a lower brightness or intensity than would normally be used to display images on mobile device display 302. As another example, the reference image 300 may be displayed using a different color, shading, outline, or any other visual effect that visually identifies the reference image 300 to a user as a background image.

According to one embodiment, a distance at which the reference image was acquired is indicated on the display of the mobile device. For example, as depicted in FIG. 3B, the distance at which the reference image was acquired may be displayed on the mobile device display 302 by “Background distance: 8 ft”, as indicated by reference numeral 304. In this example, the “Current Distance” is the current distance between the mobile device 102 and the one or more objects currently in the field of view of the camera and viewable by a user as a preview image, as described in more detail hereinafter. The background distance and/or the current distance may be indicated by other means that may vary depending upon a particular implementation, and embodiments are not limited to any particular means for indicating the background distance and the current distance. For example, the background distance and current distance may be indicated by symbols, colors, shading and other visual effects on mobile device display 302.

In step 206, one or more preview images are displayed of one or more objects currently in the field of view of the camera. For example, image acquisition application 128 may cause one or more preview images to be acquired and displayed on display 120. In FIG. 3C, a preview image 310 is displayed on the mobile device display 302. Embodiments are described herein in the context of displaying a single preview image 310 for purposes of explanation only and multiple preview images may be displayed, as described in more detail hereafter. According to one embodiment, the preview image 310 is displayed in a manner to be visually discernable by a user from the reference image 300 displayed as a background image. For example, the preview image 310 may be displayed on the mobile device display 302 using normal intensity, brightness, color, shading, outline, other special effects, etc. Displaying the preview image 310 simultaneously with the reference image 300 displayed as a background image allows a user to visually discern any differences between the distance, height and angle at which the reference image was acquired and the distance, height and angle of the preview image currently displayed on the mobile device display 302. For example, differences in distance may be readily discerned from differences in sizes of the one or more objects, represented in FIG. 3C by the triangle, rectangle, oval and circles in both the reference image 300 and the preview image 310. Differences in angle may be readily discerned when the one or more objects in the reference image 300 and the preview image 310 are three dimensional objects. This allows a user to move and/or orient the mobile device 102 so that the one or more objects depicted in the preview image 310 overlap, or are aligned with, the one or more objects depicted in the reference image 300. Furthermore, successive preview images 310 may be displayed on mobile device display 302, for example on a continuous basis, to allow a user to move and/or reorient the mobile device 102 so that the distance, height and angle of the one or more objects in the reference image 300 and the one or more preview images 310 are at least substantially the same. For example, as depicted in FIG. 3D, the mobile device 102 has been positioned and oriented so that the one or more objects in the reference image 300 and the one or more preview images overlap, indicating that the distance, height and angle of the one or more objects in the reference image 300 and the one or more preview images 310 are at least substantially the same.

In step 208, a determination is made of a current distance between the mobile device and the one or more objects currently in the field of view of the camera. For example, image acquisition application 128 may cause the distance detection mechanism to measure a current distance between the mobile device 102 and the one or more objects in the field of view of the camera 122. As another example, a current distance between the mobile device 102 and the one or more objects in the field of view of the camera 122 may be determined using a GPS component in mobile device 102 and a known location of the one or more objects. In this example, the GPS coordinates of the mobile device 102 may be compared to the GPS coordinates of the one or more objects to determine the current distance between the mobile device 102 and the one or more objects in the field of view of the camera 122.

In step 210, an indication is provided to a user of the mobile device whether the current distance is within a specified amount of the distance at which the reference image was acquired. For example, the image acquisition application 128 may compare the current distance between the mobile device 102 and the one or more objects, as determined in step 208, to the distance at which the reference image was acquired. The result of this comparison may be indicated to a user of the mobile device 102 in a wide variety of ways that may vary depending upon a particular implementation and embodiments are not limited to any particular manner of notification. For example, the image acquisition application 128 may visually indicate on the display 120 whether the current distance is within a specified amount of the distance at which the reference image was acquired. This may include, for example, displaying one or more icons on display 120 and/or changing one or more visual attributes of icons displayed on display 120. As one example, icon 306 may be displayed in red when the current distance is not within the specified amount of the distance at which the reference image was acquired, displayed in yellow when the current distance is close to being within the specified amount of the distance at which the reference image was acquired and displayed in green when the current distance is within the specified amount of the distance at which the reference image was acquired. As another example, an icon, such as a circle may be displayed and the diameter reduced as the current distance approaches the specified amount of the distance at which the reference image was acquired. The diameter of the circle may increase as the difference between the current distance and distance at which the reference image was acquired increases, indicating that the mobile device 102 is getting farther away from the distance at which the reference image was acquired. As another example, different icons or symbols may be displayed to indicate whether the current distance is within the specified amount of the distance at which the reference image was acquired. As one example, a rectangle may be displayed when the mobile device 102 is beyond a specified distance from the distance at which the reference image was acquired and then changed to a circle as the mobile device 102 approaches the distance at which the reference image was acquired.

Image acquisition application 128 may audibly indicate whether the current distance is within a specified amount of the distance at which the reference image was acquired, for example, by generating different sounds. As one example, the mobile device 102 may generate a sequence of sounds, and the amount of time between each sound is decreased as the mobile device approaches the distance at which the reference image was acquired. The current distance between the mobile device 102 and the one or more objects in the field of view of the camera 122 may also be displayed on the display, for example, as depicted in FIGS. 3C and 3D. In this example, the current distance has changed from 9.5 ft to 8.2 ft as the user moved and/or reoriented the mobile device 102, to be closer to the 8.0 ft at which the reference image was acquired.

In step 212, a second image of the one or more objects is acquired in response to a user request. For example, in response to a user selection of a button 308, the second image of the one or more objects that are currently in the field of view is acquired. Metadata is also generated for the second image and may specify, for example, camera parameter values used to acquire the second image, and a timestamp or other data, such as a sequence identifier, that indicates a sequence in which images were acquired. According to one embodiment, the metadata for the second image includes a reference to the reference image so that the reference image and the second image can be displayed together, as described in more detail hereinafter. The reference may be in any form and may vary depending upon a particular implementation. For example, the reference may include the name or identifier of the reference image. The metadata for the reference image may also be updated to include a reference to the second image.

According to one embodiment, camera settings values used to acquire the reference image are also used to acquire the second image. This ensures, for example, that the same camera settings, such as focus, aperture, exposure time, etc., are used to acquire both the reference image and the second image. This reduces the likelihood that differences in the one or more objects in the sequence of images are attributable to different camera settings used to acquire the images, rather than actual changes in the one or more objects. Camera settings used to acquire an image may be stored in the metadata for the acquired image, for example, in metadata 148, 174.

The current distance may optionally be reacquired and recorded in association with the second image, for example, in the metadata for the second image. Alternatively, the distance at which the reference image was acquired may be used for the second image, since the current distance is within the specified amount of the distance at which the reference image was acquired.

Image data, representing the second image, and optionally the current distance, may be stored locally on mobile device, for example, in memory 142, and/or may be transmitted by mobile device 102 for storage and/or processing on one or more of application server 104, EMR system 106, other services 108 or client device 110. Image data may be transmitted to application server 104, EMR system 106, other services 108 or client device 110 using a wide variety of techniques, for example, via FTP, via email, via http POST commands, or other approaches. The transmission of image data, and the corresponding metadata, may involve the verification of credentials. For example, a user may be queried for credential information that is verified before image data may be transmitted to application server 104, EMR system 106, other services 108 or client device 110. Although the foregoing example is depicted in FIG. 2 and described in the context of acquiring a second image, embodiments are not limited to acquiring a single image using a reference image and any number of subsequent images may be acquired using a reference image as a background image. When more than one subsequent images are acquired using a reference image, the metadata for the subsequent images may include a reference to the reference image and the other subsequent images that were acquired using the reference image. For example, suppose that a second and third image were acquired using the reference image. The metadata for the second image may include a reference to the reference image and to the third image. The metadata for the third image may include a reference to the reference image and the second image. The metadata for the reference image may include no references the second and third images, a reference to the second image, a reference to the third image, or both. The reference data and timestamp data are used to display the reference image and one or more subsequent images acquired using the reference image as a background image as an ordered sequence, as described in more detail hereinafter.

### IV. MEMO AND AUDIO DATA

According to one embodiment, memorandum (memo) and/or audio data may be acquired to supplement image data. Memorandum data may be automatically acquired by data acquisition component 125, for example, by scanning encoded data associated with the one or more objects in the acquired image. For example, a user of mobile device 102 may scan a bar code or QR code attached to or otherwise associated with the one or more objects, or by scanning a bar code or QR code associated with a patient, e.g., via a patient bracelet or a patient identification card. Memorandum data may be manually specified by a user of mobile device 102, for example, by selecting from one or more specified options, e.g., via pull-down menus or lists, or by entering alphanumeric characters and/or character strings.

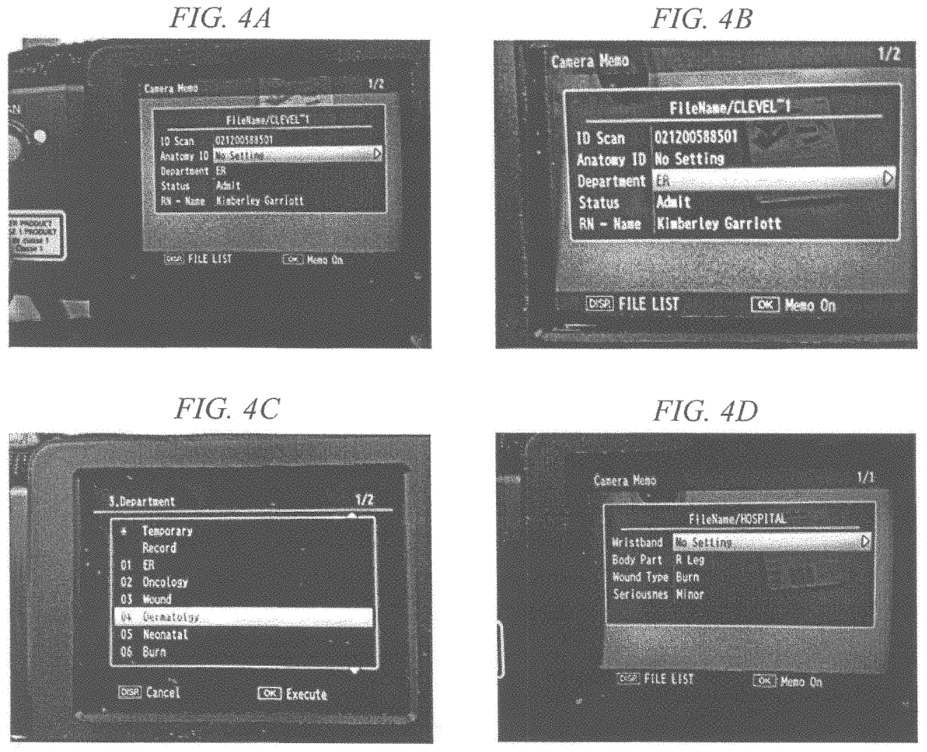

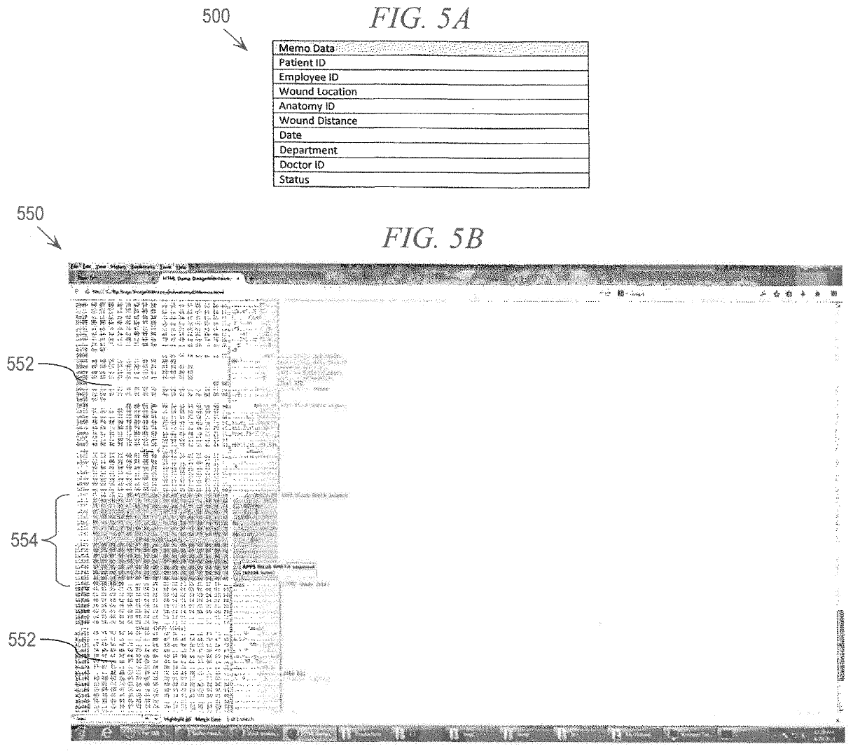

FIGS. 4A-D depict an example graphical user interface displayed on display 120 of mobile device 102 that allows a user to specify memorandum data in a medical context. The graphical user interface may be generated, for example, by image acquisition application 128. FIG. 4A depicts top-level information that includes a patient identification field (“ID Scan”), an anatomy identification field (“Anatomy ID”), a department field (“Department”), a status field (“Status”) and a registered nurse name (“RN—Name”). FIG. 4B depicts that a user has used one or more controls (graphical or physical) on mobile device 102 to navigate to the department field. FIG. 4C depicts the department options available to the user after selecting the department field and that the user has navigated to the Dermatology department option. In FIG. 4D, the graphical user interface allows the user to specify a wristband setting, a body part, a wound type and an indication of the seriousness of the injury.

FIG. 5A depicts a table 500 of example types of memorandum data. Although embodiments are described in the context of example types of memorandum data for purposes of explanation, embodiments are not limited to any particular types of memorandum data. In the example table 500 depicted in FIG. 5A, the memorandum data is in the context of images of a human wound site and includes a patient ID, an employee ID, a wound location, an anatomy ID, a wound distance, i.e., a distance between the camera 122 and the wound site, a date, a department, a doctor ID and a status.

Audio data may be acquired, for example, by image acquisition application 128 invoking functionality provided by operating system 136 and/or other applications 126 and microphone 130. The acquisition of audio data may be initiated by user selection of a graphical user interface control or other control on mobile device 102. For example, a user may initiate the acquisition of audio data at or around the time of acquiring one or more images to supplement the one or more images. As described in more detail hereinafter, audio data may be processed by transcription application 166 to provide an alphanumeric representation of the audio data.

Memorandum data and/or audio data may be stored locally on mobile device, for example, in memory 142, and/or may be transmitted by mobile device 102 for storage and/or processing on one or more of application server 104, EMR system 106, other services 108 or client device 110. Memorandum data may be stored as part of metadata 148, 174. Audio data may be stored locally on mobile device 102 as audio data 146 and on application server 104 as audio data 172. In addition, memorandum data and/or audio data may be transmitted separate from or with image data, e.g., as an attachment, embedded, etc.

FIG. 5B is a table 550 that depicts a textual representation of image data 552 that includes embedded audio data 554. In this example, audio data 146, 172 is stored as part of image data 144, 170. Memorandum data may similarly be embedded in image data. The way in which memorandum data and audio data is stored may vary from image data to image data and not all memorandum data and audio data must be stored in the same manner. For example, audio data that corresponds to a reference image may be embedded in the image data for the reference image, while audio data that corresponds to a second image may be stored separate from the image data for the second image.

### V. IMAGE DATA MANAGEMENT

Various approaches are provided for managing image data. According to one embodiment, image management application 164 provides a user interface for managing image data. The user interface may be implemented, for example, as a Web-based user interface. In this example, a client device, such as client device 110, accesses image management application 164 and the user interface is implemented by one or more Web pages provided by image management application 164 to client device 110.

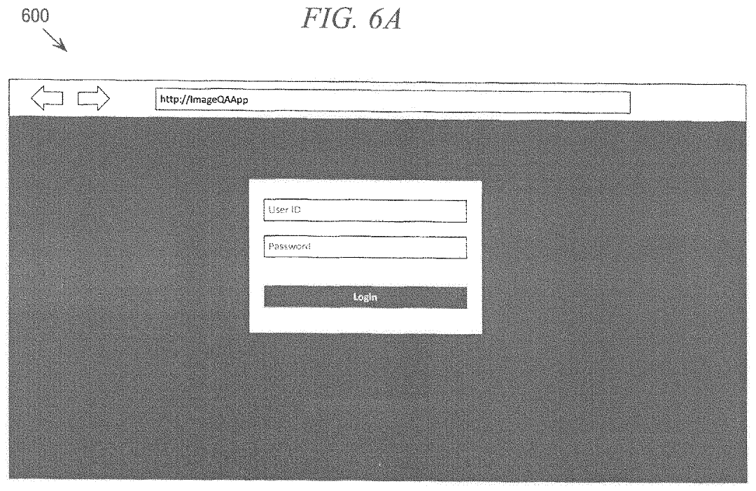

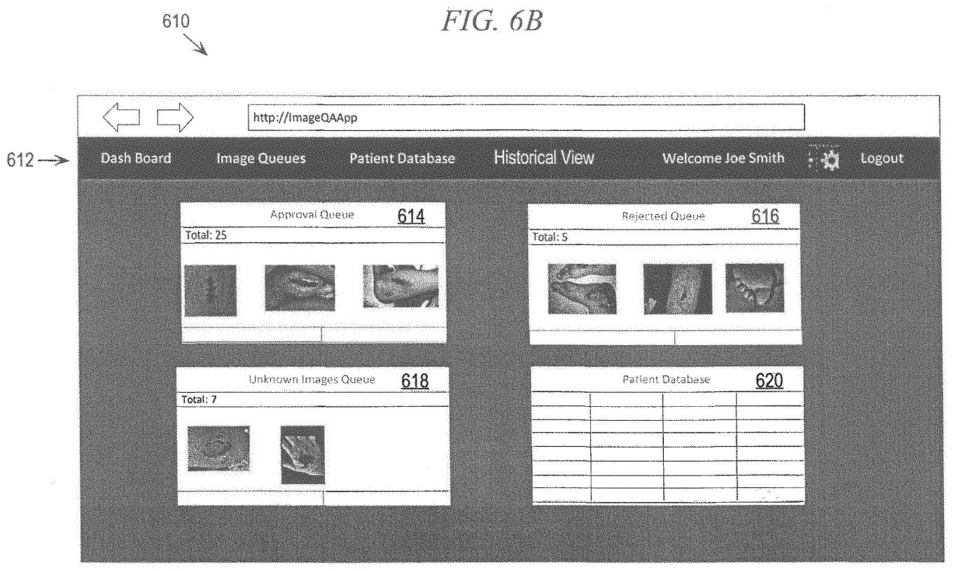

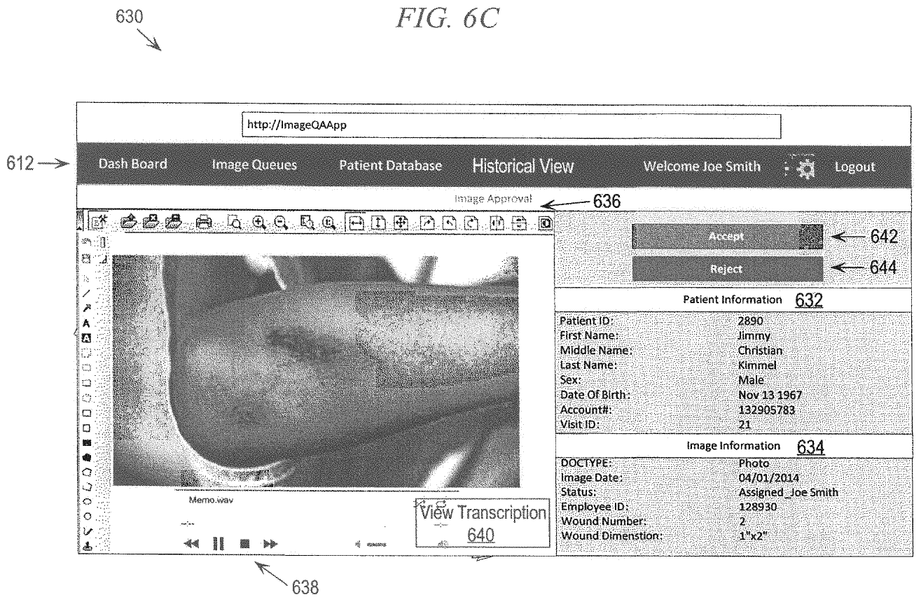

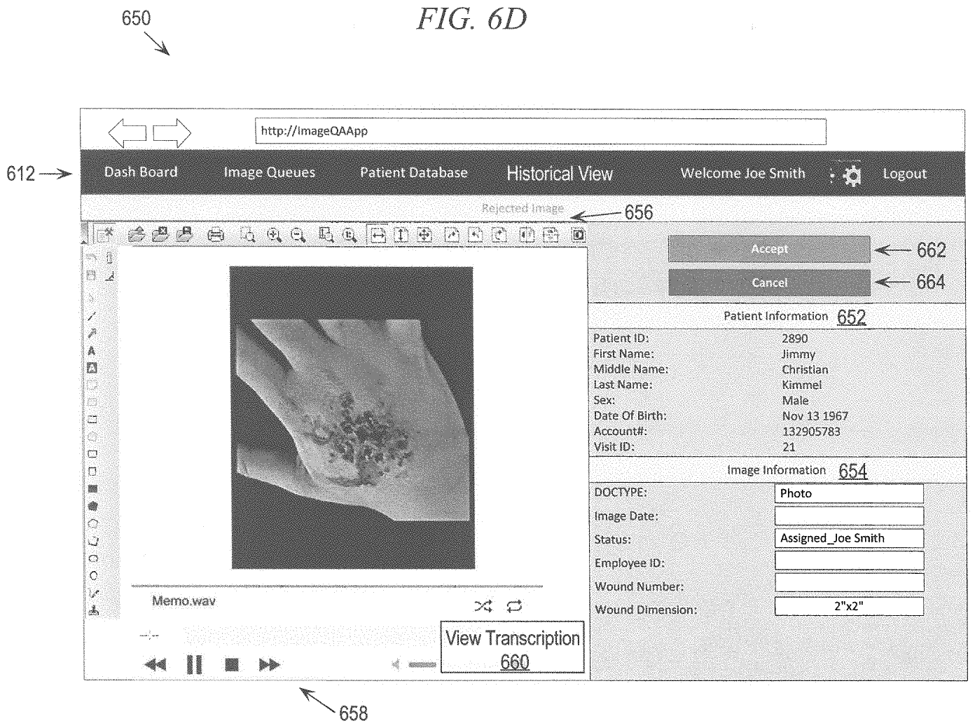

FIGS. 6A-6D depict an example graphical user interface for managing image data according to an embodiment. The example graphical user interface depicted in FIGS. 6A-6D may be provided by one or more Web pages generated on application server 104 and provided to client device 110. FIG. 6A depicts an example login screen 600 that queries a user for user credentials that include a user login ID and password.

FIG. 6B depicts an example main screen 610, referred to hereinafter as a “dashboard 610”, that provides access to various functionality for managing image data. In the example depicted in FIG. 6B, the dashboard 610 provides access, via graphical user interface controls 612, to logical collections of images referred to hereinafter as “queues,” a user database in the form of a patient database and historical views of images. Although embodiments are described hereinafter in the medical/accident context for purposes of explanation, embodiments are not limited to this context. The queues include an Approval Queue, a Rejected Queue and an Unknown Images Queue that may be accessed via graphical user interface icons 614, 616, 618, respectively. The patient database may be accessed via graphical user interface icon 620.

FIG. 6C depicts an example Approval Queue screen 630, or work queue, that allows a user to view and approve or reject images. Approval Queue screen 630 displays patient information 632 of a patient that corresponds to the displayed image and image information 634 for the displayed image. Approval Queue screen 630 includes controls 636 for managing the displayed image, for example, by expanding (horizontally or vertically) or rotating the displayed image. Controls 638 allow a user to play an audio recording that corresponds to the displayed image. Control 640 allows a user to view an alphanumeric transcription of the audio recording that corresponds to the displayed image. The alphanumeric transcription may be generated by transcription application 166 and displayed to a user in response to a user selection of control 640. Approval Queue screen 630 also includes controls 642, 644 for approving (accepting) or rejecting, respectively, the displayed image. A displayed image might be rejected for a wide variety of reasons that may vary depending upon a particular situation. For example, a user might choose to reject a displayed image because the image is out of focus, the image is otherwise of poor quality, the image does not show the area of interest, or the information associated with the image, such as the patient information 632 or the image information 634 is incomplete.

FIG. 6D depicts an example Rejected Image Processing screen 650 that allows a user to view and update information for rejected images. Rejected Image Processing screen 650 displays patient information 652 of a patient that corresponds to the displayed image and image information 654 for the displayed image. A user may correct or add to the metadata or memorandum data for the displayed image. For example, the user may correct or add to the patient information 652 or the image information 654, e.g., by selecting on a field and manually entering alphanumeric information. Rejected Image Processing screen 650 includes controls 656 for managing the displayed image, for example, by expanding (horizontally or vertically) or rotating the displayed image. Controls 658 allow a user to play an audio recording that corresponds to the displayed image. Control 660 allows a user to view an alphanumeric transcription of the audio recording that corresponds to the displayed image. Rejected Image Processing screen 650 also includes controls 662, 664 for approving (accepting) or rejecting, respectively, the displayed image. For example, after making changes to the displayed image, the patient information 652 or the image information 654, a user may select control 662 to accept the displayed image and cause the displayed image to be added to the Approval queue. Alternatively, a user may maintain the displayed image as rejected by selecting control 664 to cancel.

The unknown images queue accessed via control 618 includes images for which there are incomplete information or other problems, which may occur for a variety of reasons. For example, a particular image may have insufficient metadata to associate the particular image with other images. As another example, a particular image may be determined to not satisfy specified quality criteria, such as sharpness, brightness, etc. Users may perform processing on images in the unknown images queue to provide incomplete information and/or address problems with the images. For example, a user may edit the metadata for a particular image in the unknown images queue to supply missing data for the particular image. As another example, a user may process images in the unknown image queue to address quality issues, such as poor focus, insufficient brightness or color contrast, etc. The images may then be approved and moved to the approval queue or rejected and moved to the rejected queue.

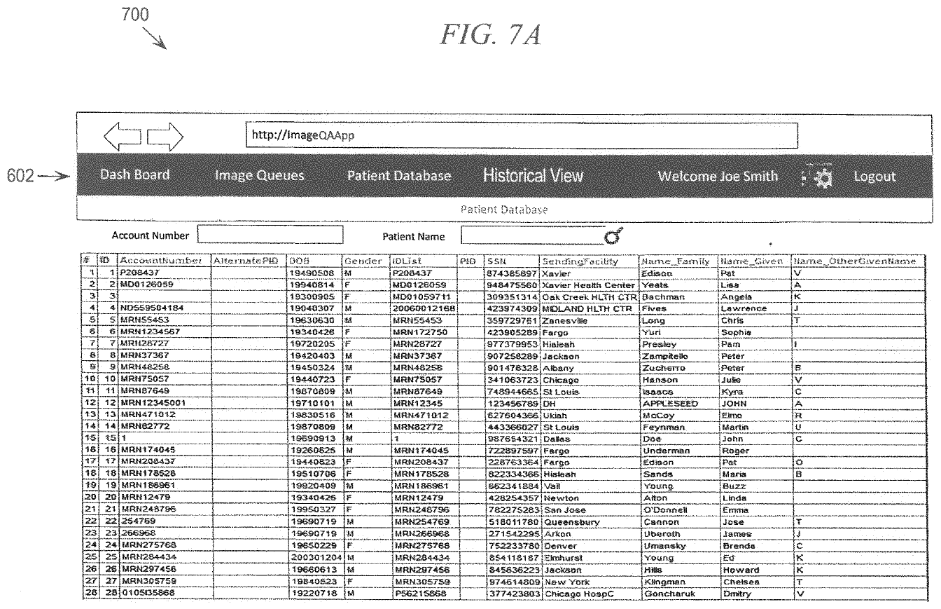

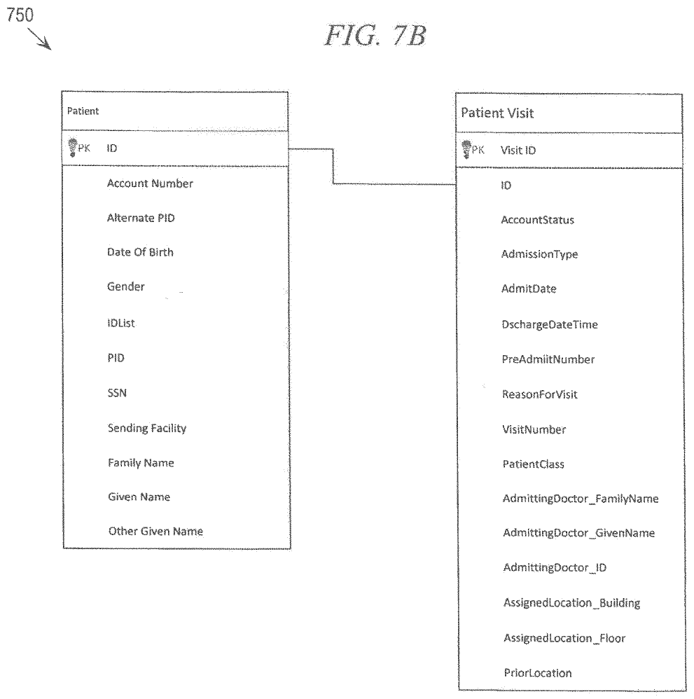

FIG. 7A is a table 700 that depicts an example patient database, where each row of the table 700 corresponds to a patient and specifies an identifier, a date of birth (DOB), a gender, an ID list, a social security number (SSN), a sending facility, a family name, a first (given) name and another given (middle) name. Table 700 may be displayed in response to a user selecting the “Patient Database” control 612. FIG. 7B is a table 750 that depicts an example patient database schema.

### VI. HISTORICAL VIEWS

According to one embodiment, images are displayed to a user using a historical view. In general, a historical view displays a sequence of images that includes a reference image and one or more other images acquired using the reference image as a background image as described herein.

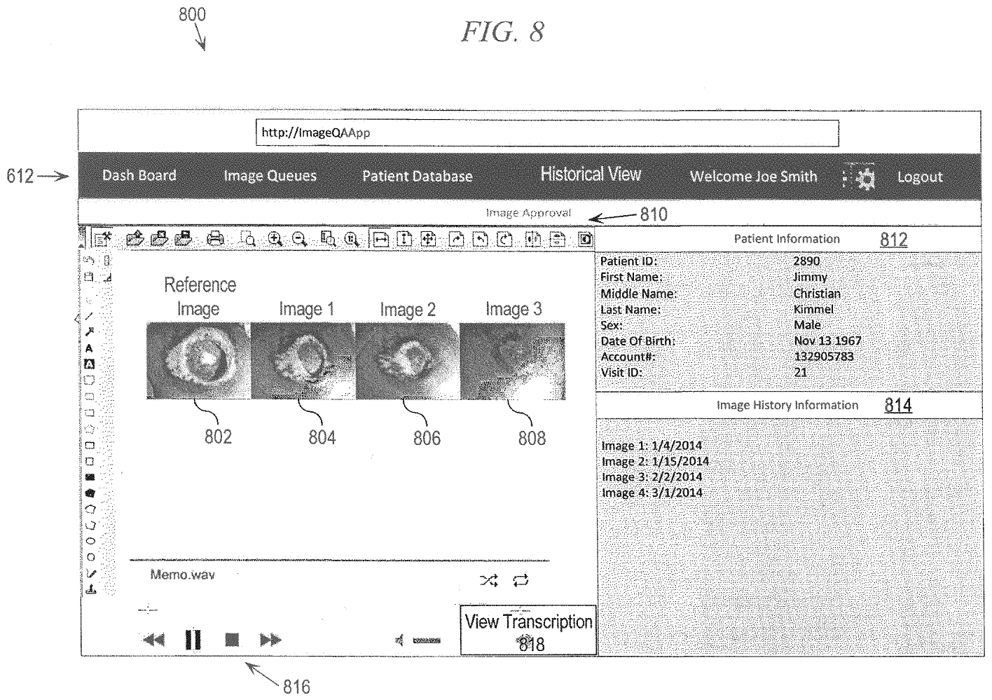

FIG. 8 depicts an example historical view screen 800 generated by image management application 164 according to an embodiment. A user of client device 110 may access image management application 164 and request access to a historical view of images, for example, by selecting the “Historical View” control 612. In response to this request, image management application 164 may provide access to historical view screen 800. As one non-limiting example, historical view screen 800 may be represented by one or more Web pages provided by image management application 164 to client device 110.

In the example depicted in FIG. 8, historical view screen 800 includes a plurality of graphical user interface objects that include graphical user interface controls 612 that provide access to the dashboard, the image queues and the patient database previously described herein. The historical view screen 800 includes a sequence of images 802-808 of one or more objects selected by a user. When the historical view screen 800 is first displayed, a user may be shown a collection of image sequences, where each image sequence is represented by one or more graphical user interface objects, such as an icon, textual description, thumbnail image or other information. The user selects a graphical user interface object, for example an icon, which corresponds to a particular image sequence of interest, and the images in the particular sequence are displayed.

One or more graphical user interface controls may be provided to arrange the image sequences by a time of information selected, e.g., user identification, organization, event, subject, date/time, etc. The graphical user interface controls may also allow a user to enter particular criteria and have the image sequences that correspond to the particular criteria be displayed. In the example depicted in FIG. 8, the images 802-808 correspond to a particular patient identified in patient information 812. Each image sequence includes the reference image and one or more subsequent images acquired using the reference image, as previously described herein. Note that in the example depicted in FIG. 8, multiple image sequences may be provided for a single user, i.e., a single patient. For example, suppose that a patient sustained injuries on two locations of their body, e.g., an arm and a leg. In this example, one image sequence may correspond to the patient's arm and another image sequence may correspond to the patient's leg.

The images 802-808 include a reference image 802 and three subsequent images acquired using the reference image 802, namely, Image 1 804, Image 2 806 and Image 3 808. In this example, Image 1 804, Image 2 806 and Image 3 808 were acquired using the reference image 802 displayed on the mobile device 102 as a background image, as previously described herein. In addition, the images 802-808 are arranged on historical view screen 800 in chronological order, based upon the timestamp or other associated metadata, starting with the reference image 802, followed by Image 1 804, Image 2 806 and Image 3 808.

Historical view screen 800 also includes controls 810 for managing displayed images 802-808 and information about a user that corresponds to the images 802-808, which in the present example is represented by patient information 812. Image history information 814 displays metadata for images 802-808. In the example depicted in FIG. 8, the metadata includes a date at which each image 802-808 was acquired, but the metadata may include other data about images 802-808, for example, a distance at which the images were acquired 802-808, timestamps, memorandum data, etc. Metadata may also be displayed near or on a displayed image. For example, the timestamp that corresponds to each image 802-808 may be superimposed on, or be displayed adjacent to, each image 802-808.

Controls 816 allow a user to play an audio recording that corresponds to the displayed image and a control 818 allows a user to view an alphanumeric transcription of the audio recording that corresponds to the displayed image.

The historical view approach for displaying a sequence of images that includes a reference image and one or more other images that were acquired using the reference image as a background image and at approximately the same distance is very beneficial to see changes over time in the one or more objects captured in the images. For example, the approach allows medical personnel to view changes over time of a wound or surgical sight. As another example, the approach allows construction personnel to monitor progress of a project, or identify potential problems, such as cracks, improper curing of concrete, etc. As yet another example, the approach allows a user to monitor changes in natural settings, for example, to detect beach or ground erosion.

### VII. MANAGING ACCESS TO IMAGES USING ROLES

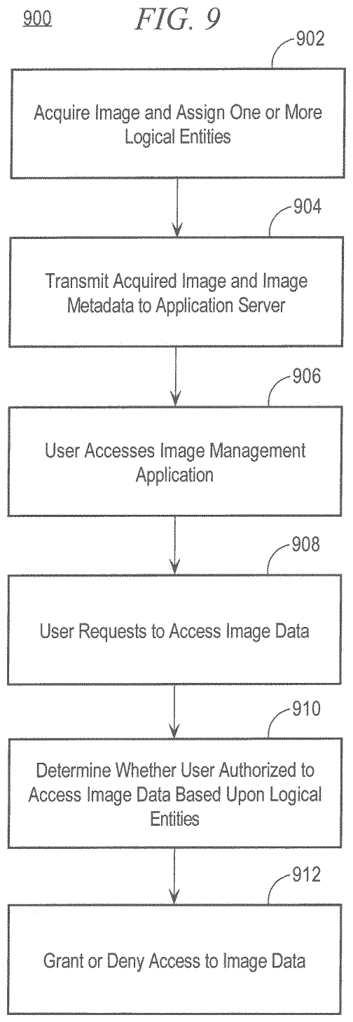

According to one embodiment, access to images acquired using mobile devices is managed using roles. Images acquired by a mobile device are assigned one or more logical entities. Users are also assigned one or more roles. The term “role” is used herein to refer to a logical entity and users may have any number of roles. As described in more detail hereinafter, a role for a user may specify one or more logical entities assigned to the user, as well as additional information for the user, such as one or more workflows assigned to the user. Users are allowed to access image data for which they have been assigned the required logical entities. The approach provides a flexible and extensible system for managing access to image data and is particularly beneficial in situations when images contain sensitive information. The approach may be used to satisfy business organization policies/procedure and legal and regulatory requirements. The approaches described herein are applicable to any type of logical entities. Examples of logical entities include, without limitation, a business organization, a division, department, group or team of a business organization. FIG. 9 is a flow diagram 900 that depicts an approach for managing access to images using logical entities. The approach of FIG. 9 is described in the context of a single image for purposes of explanation, but the approach is applicable to any number and types of images.

In step 902, an image is acquired by a client device. For example, a user of mobile device 102 may acquire an image using image acquisition application 128 and metadata for the acquired image is generated. As previously described herein, the metadata for the acquired image may specify the camera settings used to acquire the image, as well as memorandum data for the image. According to one embodiment, metadata for the acquired image specifies one or more logical entities assigned to the acquired image. The one or more logical entities may be specified in a wide variety of ways that may vary depending upon a particular implementation. For example, mobile device 102 may be configured to automatically assign one or more particular logical entities to images captured by mobile device 102. This may be useful, for example, when mobile device 102 is associated with a particular logical entity, such as a department of a business organization, so that images captured with the mobile device 102 are automatically assigned to the department of the business organization. Alternatively, logical entities may be specified by a user of the mobile device. For example, a user of mobile device 102 may manually specify one or more logical entities to be assigned to a captured image. This may be accomplished by the user selecting particular logical entities from a list of available logical entities. For example, image acquisition application 128 may provide graphical user interface (GUI) controls for selecting logical entities. As another example, mobile device 102 may include manual controls that can be used to select logical entities. Alternatively, a user may manually enter data, such as the names, IDs, etc., of one or more logical item groups to be assigned to an acquired image. As another example, a user of a mobile device may use the mobile device to scan encoded data to assign one or more logical groups to an acquired image. For example, a user may use data acquisition mechanism 125 of mobile device 102 to scan encoded data that corresponds to one or more logical entities. Logical entities may be assigned to images in a similar manner for other types of image acquisition devices. For example, images acquired by a scanning device, MFP or camera may be assigned logical entities by a user of the scanning device, MFP or camera, e.g., via a graphical user interface or controls provided by the scanning device, MFP or camera.

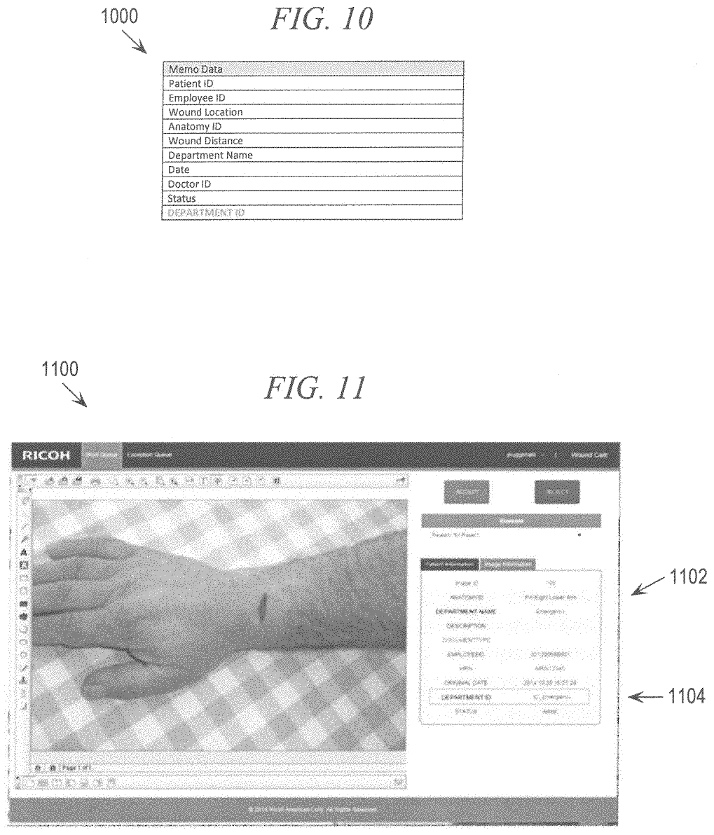

FIG. 10 depicts a table 1000 of example types of memorandum data that may be included in the metadata for an image. Although embodiments are described in the context of example types of memorandum data for purposes of explanation, embodiments are not limited to any particular types of memorandum data. In the example table 1000 depicted in FIG. 10, the memorandum data is in the context of images of a human wound site and includes a patient ID, an employee ID, a wound location, an anatomy ID, a wound distance, i.e., a distance between the camera 122 and the wound site, a date, a department name, a doctor ID, a status, and a logical entity in the form of a department ID. The department ID field of the memorandum data depicted in FIG. 10 may specify any number of departments. For example, the department ID field may specify an emergency room department as “ID_ER” or a pediatrics department as “ID_Pediatrics.”

In step 904, the acquired image and metadata for the acquired image are transmitted to application server 104. For example, image acquisition application 128 on mobile device 102 may cause the acquired image and corresponding metadata to be transmitted to application server 104 and stored in storage 168. The location where the image data and metadata are stored may be automatically configured in mobile device 102 or the location may be specified by a user, for example, by selecting one or more locations via a GUI displayed by image acquisition application 128. Image data and metadata may be immediately transmitted to application server 104 as soon as the image data and metadata are acquired. Alternatively, image data and metadata may be stored locally on mobile device 102 and transmitted to application server 104 when requested by a user. This may allow a user an opportunity to select particular images, and their corresponding metadata, that are to be transmitted to application server 104.

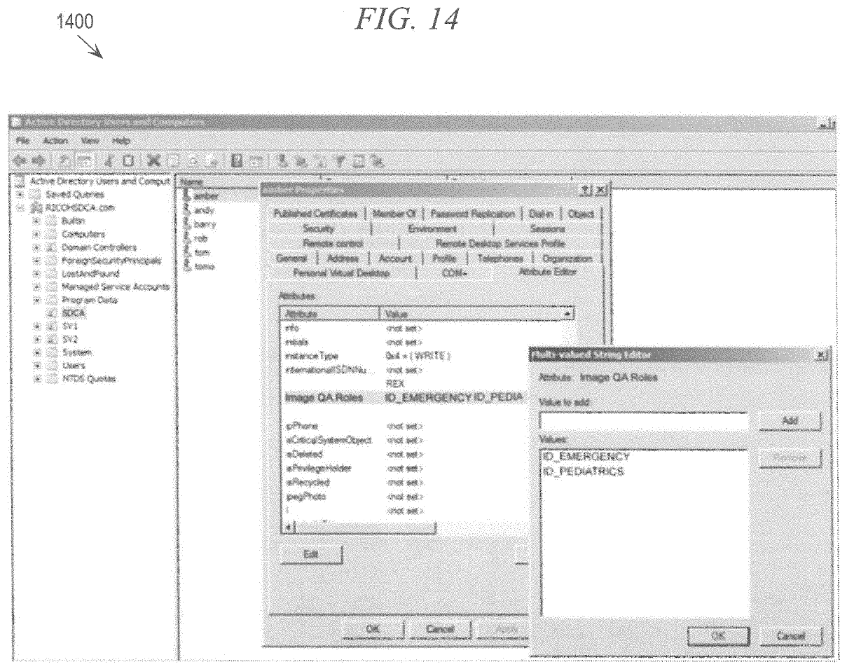

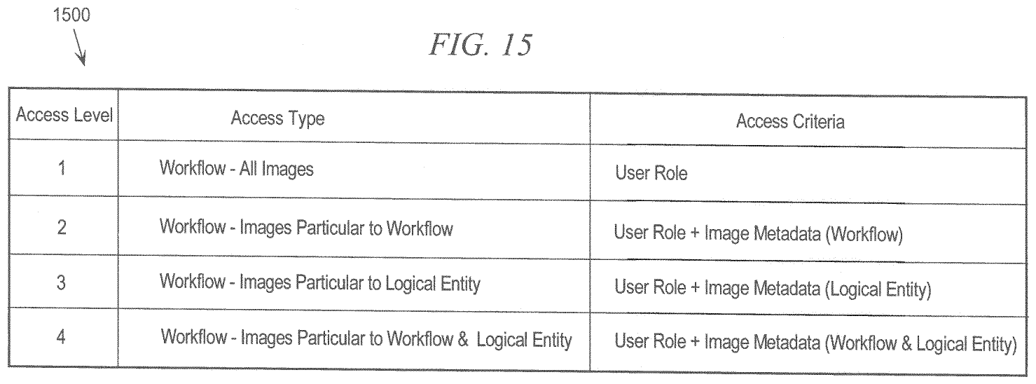

In step 906, a user wishing to view images acquired by mobile device 102 accesses image management application 164. For example, a user of client device 110 accesses image management application 164 on application server 104. The user of client device 110 may be the same user that acquired the images using mobile device 102, or a different user. As previously described herein, users may be required to be authenticated before being allowed to access image management application 164. For example, as depicted later herein with respect to FIG. 14, in the context of a system that implements Active Directory, a user requesting access to image management application 164 may be queried for user credentials and the Active Directory determines, based upon the user credentials, whether the user is a normal user or an administrator. The authentication required to access image management application 164 to specify roles, i.e., logical entities, for users may be different than the authentication required to access EMR system 106.

In step 908, the user requests to access image data. As previously described herein, users may access images in a wide variety of ways, e.g., via dashboard 610 to access logical collections of images, such as Approval Queue, Rejected Queue, Unknown Queue, etc.

In step 910, a determination is made whether the user is authorized to access the requested image data using logical entities. According to one embodiment, this includes determining one or more roles, i.e., logical entities, assigned to the user and determining one or more logical entities assigned to the image data that the user requested to access. The determination whether the user is authorized to access the requested image data is then made based upon the one or more roles, i.e., logical entities, assigned to the user and the one or more logical entities assigned to the image data that the user requested to access. Consider an example in which a particular image has been acquired via mobile device 102 and stored on application server 104, and a particular user wishes to access the particular image. After being authenticated to access image management application 164 and requesting access to the particular image, one or more roles, i.e., logical entities, assigned to the user and one or more logical entities assigned to the particular image are determined. According to one embodiment, if any of the one or one or more roles, i.e., logical entities, assigned to the user match the one or more logical entities assigned to the particular image, then the user is granted access to the particular image. For example, suppose that the particular image has been assigned the logical entities “Emergency Room” and “Pediatrics.” In this example, if the particular user has been assigned either the role, i.e., logical entity, “Emergency Room” or “Pediatrics,” then in step 912, the user is granted access to the particular image. Otherwise, in step 912, the user is not granted access to the particular image.

FIG. 11 depicts an example GUI screen 1100 after a user has been granted access to a requested image. In this example, GUI screen 1100 includes information 1102 about the image. The information 1102 may include data from the metadata for the image, such as memorandum data. The information 1102 includes a logical entity in the form of a Department ID assigned to the image which, in the present example, is “ID_EMERGENCY.” According to one embodiment, the logical entities assigned to images may be changed. For example, image management application 164 may provide an administrative GUI for adding, editing and deleting logical entities assigned to images.

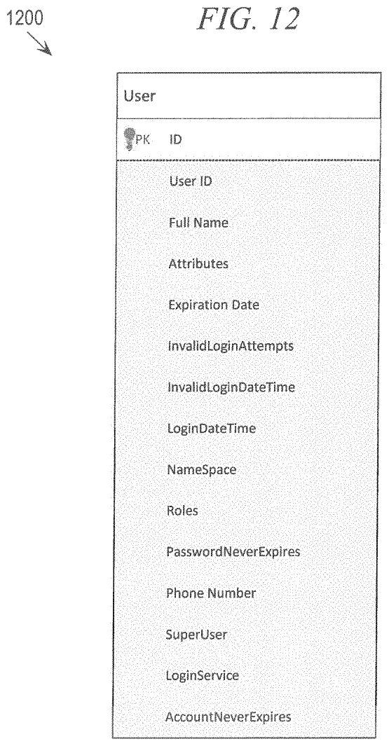

FIG. 12 depicts an example user table schema 1200 that defines an example data schema for users. In this example, the user data includes a user ID, a full name, one or more attributes of the user, an expiration date, invalid login attempts, invalid login dates and times, login dates and times, a namespace, one or more roles, data indicating whether the user's password never expires, a phone number, data indicating whether the user is a super user, a login service and data indicating whether the user's account never expires. As previously described herein, the roles for a user may specify one or more logical entities assigned to the user, as well as additional information, such as one or more workflows. Additional data, or less data, may be included in a user table schema, depending upon a particular implementation, and embodiments are not limited to the data depicted in the example user table schema of FIG. 12.

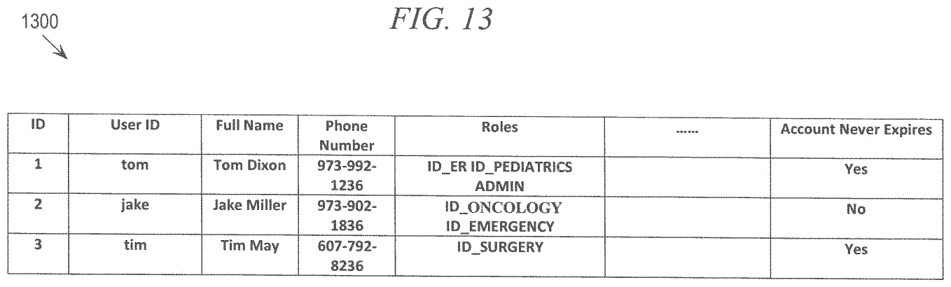

FIG. 13 depicts an example user table 1300 that specifies various types of user data. More specifically, in user table 1300, each row corresponds to a user and each column specifies a value for a data type. The columns may correspond to the data types depicted in the user table schema 1200 of FIG. 12. In the example depicted in FIG. 13, the data types include a user ID, a full name, a phone number, roles, one or more other data types, and whether the account never expires. The full name is the full name of the user, the phone number is the phone number of the user and the account never expires specifies whether the account of the user never expires. The roles specify the roles, i.e., logical entities, assigned to the user. In the example depicted in FIG. 13, the user corresponding to the first row of the user table 1300 has assigned roles of “ID_ER”, “ID_PEDIATRICS” and “ADMIN,” which may correspond to the emergency room and pediatrics departments of a business organization, such as a medical provider. The assigned role of “ADMIN” may permit the user to have administrative privileges with respect to application server 104. This user will therefore be allowed to access images associated with the emergency room and pediatrics departments in the business organization, and is also allowed to perform various administrative functions on application server 104. In contrast, the user corresponding to the third row of the user table 1300 has a single assigned role of “ID_SURGERY,” which may correspond to a surgery department within a business organization, such as a medical provider.

User data may be stored on application server 104, for example, in user data 176 on storage 168. Alternatively, user data may be stored remotely with respect to application server 104 and accessed by image management application 164, for example, via network 112. User data 176 may be managed by image management application 164 and according to one embodiment, image management application 164 provides a user interface that allows users, such as an administrator, to define and update user data. FIG. 14 depicts an example GUI 1400 for specifying user data. In the example depicted in FIG. 14, the GUI 1400 provides a window 1402 that allows a user to specify roles, i.e., logical entities, for a user. In this example, the roles of “ID_EMERGENCY” and “ID_PEDIATRICS” have already been defined for user “amber” and additional roles may be specified.

### VIII. MANAGING ACCESS TO WORKFLOWS USING ROLES

According to one embodiment, access to workflows to process images acquired using mobile devices is managed using roles. The term “workflow” is used herein to refer to a process for processing images acquired by mobile devices and the processes may be provided, for example, by image management application 164. Example processes include, without limitation, processes for approving, rejecting and updating images, and viewing historical views of images, as described herein. Users are authorized to access particular workflows, as specified by user data. When a particular user requests access to a particular process for processing images acquired by mobile devices, a determination is made, based upon the user data for the user, whether the user is authorized to access the particular process to process images acquired by mobile devices. The user is granted or not granted access based upon the determination.

Further access control may be provided using roles. More specifically, user data and roles may be used to limit access by a user to a particular workflow and particular images. For example, as described in more detail hereinafter, a request for a user to process a particular image using a particular workflow (or a request to access the particular workflow to process the particular image) may be verified based upon both whether the user is authorized to access the particular workflow and whether the user is authorized to access the particular image. In addition, workflow levels may be used to manage access to particular functionality within a workflow. Thus, different levels of access granularity may be provided, depending upon a particular implementation.

A. Access Levels

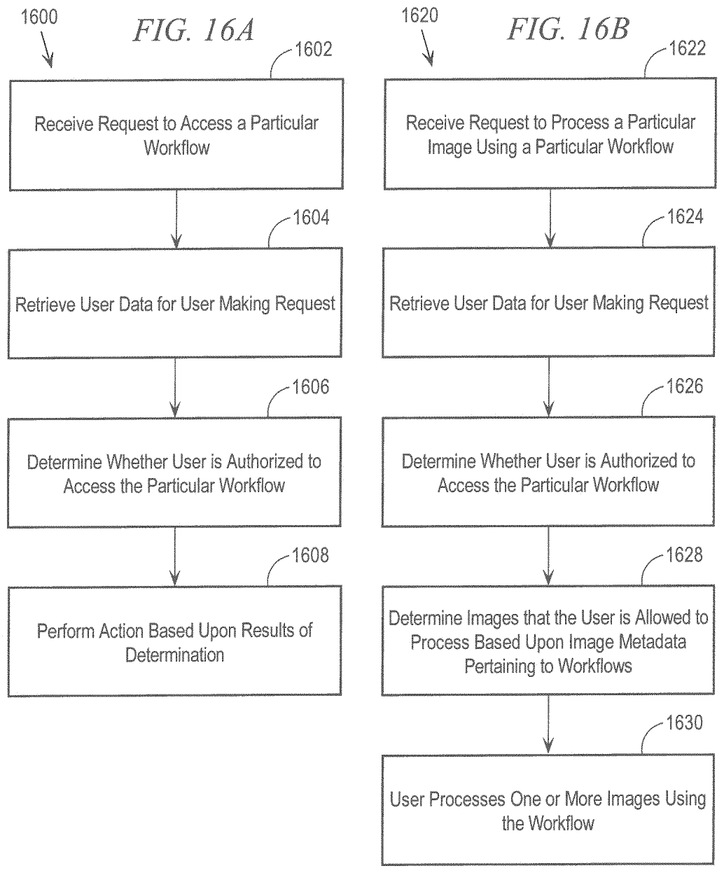

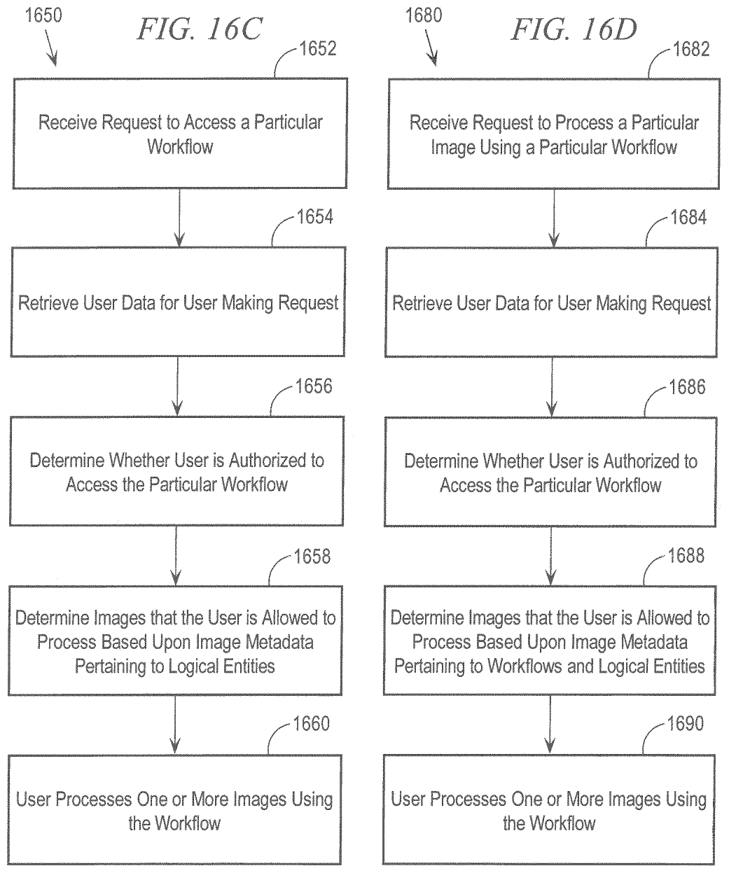

FIG. 15 is a table 1500 that depicts four example levels of access to workflows and images. The example levels of access depicted in FIG. 15 represent a hierarchy of access management, with the level of access control generally increasing from Level 1 to Level 4. In Level 1, a user is granted access to a particular workflow and is able to process any images with the particular workflow. For example, a user may be granted access to a process for viewing and approving or rejecting images, as previously described herein. This example process is used as an example workflow for describing FIG. 15 and FIGS. 16A-16D. For Level 1, the user's role, and more particularly the processes that the user is authorized to access, are used as the access criteria, as indicated by the user data 176 for the user. In this example, the user data 176 for the user must specify that the user is authorized to access the process for viewing and approving or rejecting images.

FIG. 16A is a flow diagram 1600 that depicts an approach for managing access to a workflow using the access criteria for Level 1. In step 1602, a request is received to access a particular workflow, which in the present example is the process for viewing and approving or rejecting images, as previously described herein. For example, a user of client device 110 may access a GUI provided by image management application 164 and request to access the process to view and approve or reject images. In step 1604, user data for the user making the request is retrieved. For example, image management application 164 may retrieve user data 176 for the user requesting to access the process provided by image management application 164 for viewing and approving or rejecting images. In step 1606, a determination is made whether the user is authorized to access the particular workflow, i.e., the process to view and approve or reject images. For example, image management application 164 may determine, based upon the user data 176 for the user, whether the user is authorized to access the process provided by image management application 164 for viewing and approving or rejecting images. The user data 176 for the user may specify by name, ID, etc., one or more processes that the user is authorized to access. In step 1608, one or more actions are performed based upon the results of the determination in step 1606. For example, the user may be granted or denied access to the process provided by image management application 164 for viewing and approving or rejecting images.

In Level 2, a user is granted access to a particular workflow and images that are particular to the workflow. Level 2 differs from Level 1 in that a user is not granted access to all images using the workflow, but only images that are particular to the workflow. For example, a user may be granted access to the process for viewing and approving or rejecting images, but only with respect to images that are particular to the particular workflow. For Level 2, the user's role and image metadata, pertaining to associated workflows, are used as access criteria. More specifically, the user's data must specify that the user is authorized to access the particular workflow and also the metadata for the images must specify that the images are associated with the particular workflow. In this example, the user data 176 for the user must specify that the user is authorized to access the process for viewing and approving or rejecting images and the metadata for the images must specify that the images are associated with the process for viewing and approving or rejecting images. Access is not allowed for images that are not associated with the particular workflow.

FIG. 16B is a flow diagram 1620 that depicts an approach for managing access to a workflow using the access criteria for Level 2. In step 1622, a request is received to access the process for viewing and approving or rejecting images. In step 1624, user data for the user making the request is retrieved and in step 1626, a determination is made whether the user is authorized to access the process to view and approve or reject images, as previously described herein. Assuming that the user is authorized to access the process to view and approve or reject images, then in step 1628, a determination is made of the images that the user is allowed to process using the process to view and approve or reject images. For Level 2, this includes examining image metadata to identify images that are associated with the process to view and approve or reject images. In step 1630, the user processes one or more of the available images using the process provided by image management application 164 for viewing and approving or rejecting images.

In Level 3, a user is granted access to a particular workflow and images that are particular to logical entities that the user is allowed to access. For example, a user may be granted access to a process for viewing and approving or rejecting images, but only with respect to images that are particular to a particular logical entity, such as a department within a business organization, that the user is authorized to access. For Level 3, the user's role and image metadata, pertaining to logical entities, are used as access criteria. More specifically, the user's data must specify that the user is authorized to access the particular workflow and a particular logical entity, e.g., a particular department of a business organization. Also, the metadata for the images must specify that the images are associated with the specified logical entity. In this example, the user data 176 for the user must specify that the user is authorized to access the process for viewing and approving or rejecting images and is authorized to access images for the particular department of the business organization. The metadata for the images must specify that the images are associated with the department within the business organization. Unlike Level 2, the images are not required to be associated with the workflow, i.e., the process for viewing and approving or rejecting images. Access is not allowed, however, for images that are not associated with the particular logical entity, i.e., the department within the business organization, that the user is authorized to access.

FIG. 16C is a flow diagram 1650 that depicts an approach for managing access to a workflow using the access criteria for Level 3. In step 1522, a request is received to access the process for viewing and approving or rejecting images. In step 1654, user data for the user making the request is retrieved and in step 1656, a determination is made whether the user is authorized to access the process to view and approve or reject images, as previously described herein. Assuming that the user is authorized to access the process to view and approve or reject images, then in step 1658, a determination is made of the images that the user is allowed to process using the process to view and approve or reject images. For Level 3, this includes examining the user data for the user to determine one or more logical entities assigned to the user. Image metadata is also examined to identify images that are associated with the one or more logical entities assigned to the user. For example, suppose that the user is assigned to a particular department within a business organization. In this example, the user is allowed to use the particular process to process images that are associated with the particular department within the business organization. Note that the images are not required to be associated with the workflow, i.e., the process for viewing and approving or rejecting images. In step 1630, the user processes one or more of the available images using the process provided by image management application 164 for viewing and approving or rejecting images.

In Level 4, a user is granted access to a particular workflow and images that are particular to both the particular workflow and logical entities that the user is allowed to access. For example, a user may be granted access to the process for viewing and approving or rejecting images, but only with respect to images that are particular to both the process for viewing and approving or rejection images and a logical entity, such as a department within a business organization, that is assigned to the user. For Level 4, the user's role and image metadata pertaining to associated workflows and logical entities are used as access criteria. More specifically, the user's data must specify that the user is authorized to access the particular workflow and one or more logical entities. The metadata for the images must specify that the images are associated with both the particular workflow and the one or more logical entities assigned to the user. Access is not allowed for images that are not associated with both the particular workflow and the one or more logical entities assigned to the user.

FIG. 16D is a flow diagram 1680 that depicts an approach for managing access to a workflow using the access criteria for Level 4. In step 1682, a request is received to access the process for viewing and approving or rejecting images. In step 1684, user data for the user making the request is retrieved and in step 1686, a determination is made whether the user is authorized to access the process to view and approve or reject images, as previously described herein. Assuming that the user is authorized to access the process to view and approve or reject images, then in step 1688, a determination is made of the images that the user is allowed to process using the process to view and approve or reject images. For Level 4, this includes examining the user data for the user to determine one or more logical entities assigned to the user. Image metadata is also examined to identify images that are associated with both the particular workflow, i.e., the process to view and approve or reject images, and the one or more logical entities assigned to the user. In step 1690, the user processes one or more of the available images using the process provided by image management application 164 for viewing and approving or rejecting images.

The foregoing examples are depicted and described in the context of accessing a particular workflow, i.e., a process for processing images acquired by mobile device 102, but embodiments are not limited to these example processes and are applicable to any types of processes. In addition, the approach is applicable to workflows implemented by other processes implemented application server 104 and also remote to application server 104. In this context, image management application 164 may act as a gatekeeper to processes executing remote to image management application 164.

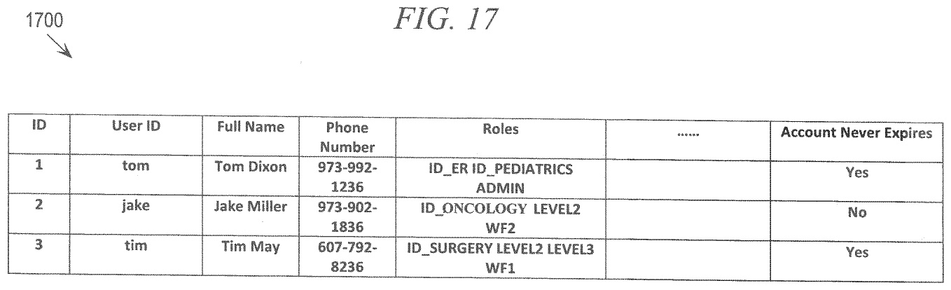

FIG. 17 depicts an example user table 1700 that specifies various types of user data. More specifically, in user table 1700, each row corresponds to a user and each column specifies a value for a data type. The columns may correspond to the data types depicted in the user table schema 1200 of FIG. 12. In the example depicted in FIG. 17, the data types include a user ID, a full name, a phone number, roles, one or more other data types, and whether the account never expires. The full name is the full name of the user, the phone number is the phone number of the user and the account never expires specifies whether the account of the user never expires. The roles specify the roles, i.e., logical entities and workflows, assigned to the user. In the example depicted in FIG. 17, the user corresponding to the first row of the user table 1700 has assigned roles of “ID_ER”, “ID_PEDIATRICS” and “ADMIN,” which may correspond to the emergency room and pediatrics departments of a business organization, such as a medical provider. The assigned role of “ADMIN” may permit the user to have administrative privileges with respect to application server 104. This user will therefore be allowed to access images associated with the emergency room and pediatrics departments in the business organization, and is also allowed to perform various administrative functions on application server 104. In contrast, the user corresponding to the third row of the user table 1300 has a single assigned role of “ID_SURGERY,” which may correspond to a surgery department within a business organization, such as a medical provider. The user corresponding to the first row of the user table 1700 does not have any assigned workflows, but the user corresponding to the second row of user table 1700 is assigned a workflow identified as “WF2” and the user corresponding to the third row of user table 1700 is assigned a workflow identified as “WF1”. In addition, the user data in user table 1700 specifies levels within workflows. Specifically, the user corresponding to the second row of user table 1700 is assigned “Level 2” of the workflow identified as “WF2” and the user corresponding to the third row of user table 1700 is assigned “Level 3” a workflow identified as “WF1”. The use of levels within workflows provides additional granularity with respect to managing access to workflows, as described in more detail hereinafter.

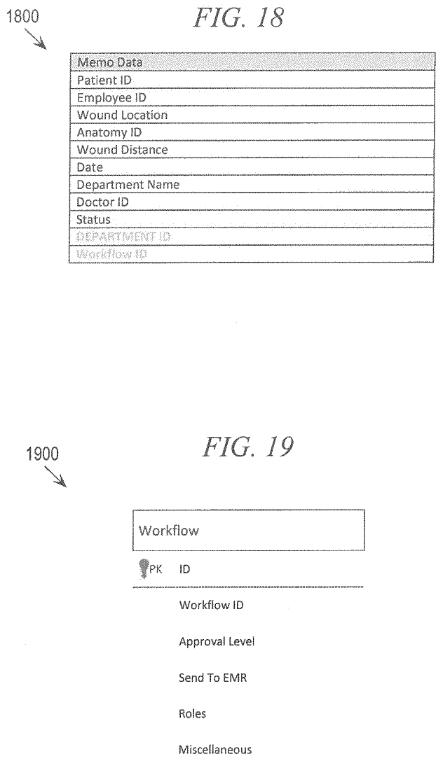

FIG. 18 depicts a table 1800 of example types of memorandum data that may be included in the metadata for an image. Although embodiments are described in the context of example types of memorandum data for purposes of explanation, embodiments are not limited to any particular types of memorandum data. In the example table 1800 depicted in FIG. 18, the memorandum data is in the context of images of a human wound site and includes a patient ID, an employee ID, a wound location, an anatomy ID, a wound distance, i.e., a distance between the camera 122 and the wound site, a date, a department name, a doctor ID, a status, a logical entity in the form of a department ID and a workflow identified by a workflow ID. The department ID field of the memorandum data depicted in FIG. 18 may specify any number of departments. For example, the department ID field may specify an emergency room department as “ID_ER” or a pediatrics department as “ID_Pediatrics.” The workflow ID field of the memorandum data depicted in FIG. 18 may specify any number of workflows. For example, the workflow ID field may specify a first workflow by “WF1” and a second workflow by “WF2”. The workflow ID field may also specify workflow levels, for example, by “Level 3” or “Level 2”.

FIG. 19 depicts an example workflow schema 1900 that defines an example data schema for workflows. In this example, the workflow data includes a workflow ID, an approval level, a send to EMR data value, roles and miscellaneous data values. The workflow ID is data that uniquely identifies a workflow. The approval level is data that indicates a level of approval required to use the workflow. The send to EMR data value indicates whether the results of the workflow should be sent to EMR system 106. The roles data value indicates one or more logical entities assigned to the workflow. For example, a workflow may be assigned to a particular department within a business organization. The miscellaneous data values may be other miscellaneous data associated with a workflow and the particular data values may vary, depending upon a particular implementation.

B. Workflow Levels

According to one embodiment, a workflow may have any number of workflow levels, where each workflow level represents a part of the workflow process. Workflow levels provide additional granularity for managing access to workflows because users may be given selective access to some workflow levels within a workflow, but not other workflow levels in the same workflow. For example, as previously described herein with respect to FIG. 17, user data may define the workflows and workflow levels assigned to particular users and the workflows and/or workflow levels assigned to users may be changed over time, e.g., by an administrator.

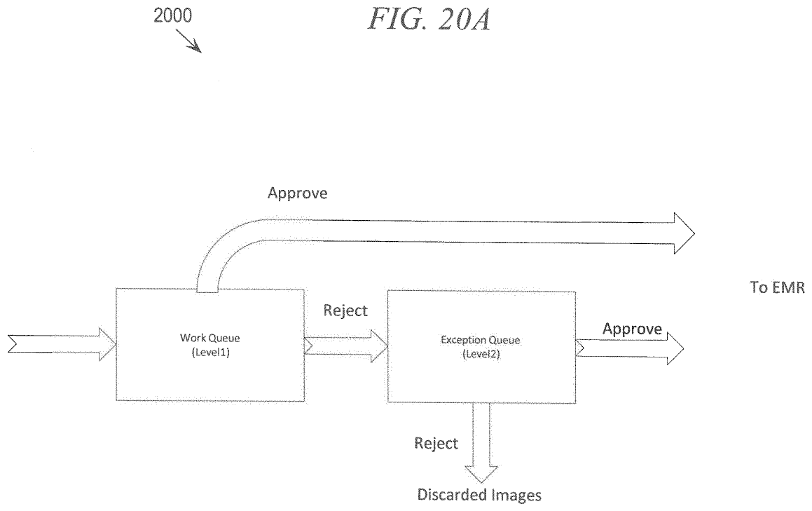

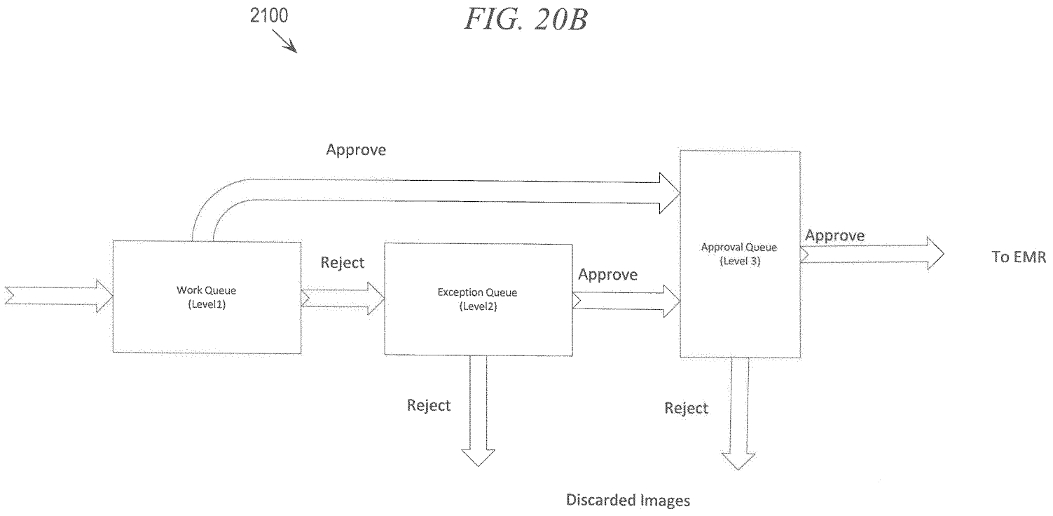

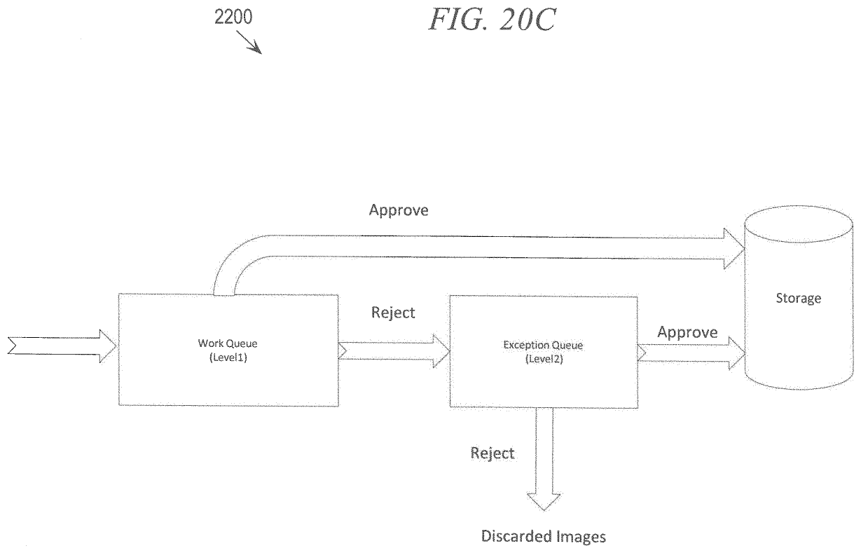

FIG. 20A depicts an example workflow 2000 for processing images. At Level 1 of workflow 2000, an image from a Work Queue is evaluated and either approved or rejected. For example, as previously described herein, image management application 164 may provide a graphical user interface that allows a user to view, from a Work Queue, images and their associated metadata, and approve or reject the images. Approved images are provided to an external system, such as EMR system 106. Rejected images are provided to an Exception Queue at Level 2 of workflow 2000 for further evaluation and/or correction. For example, an image and/or the metadata for an image may be changed or updated to correct any identified errors or to provide any missing or incomplete information. Images that are again rejected at Level 2 of workflow 200 are discarded, while images that are approved are provided to an external system, such as EMR system 106. Different levels of access may be required for Level 1 and Level 2 of workflow 200. For example, a first level of access may be required to approve or reject images in the Work Queue at Level 1, while a second and higher level of access may be required to reject or approve images in the Exception Queue at Level 2. The higher level of access may be required for Level 2, since images rejected at Level 2 are discarded.

FIG. 20B depicts an example workflow 2100 that includes all of the elements of workflow 2000 of FIG. 20A, and also includes an additional Approval Queue at Level 3 of workflow 2100. In workflow 2100, images that are approved either at the Work Queue at Level 1, or the Exception Queue at Level 2, are transmitted to an Approval Queue at Level 3. Images approved at the Approval Queue at Level 3 are transmitted to EMR system 106 and images that are rejected are discarded. The additional Approval Queue at Level 3 of workflow 2100 provides an additional level of approval that is useful in many situations, for example, when images contain sensitive information, for regulatory compliance, legal compliance, etc. A user authorized to provide the second approval of images at the Approval Queue at Level 3, may be specially-designated personnel, senior personnel, or other users authorized to provide the approval of images that will result in approved images being transmitted to EMR 106. The use of workflow levels provides great flexibility in the processing of images. For example, a first user having a first level of authority may be given access to the Work Queue at Level 1, but not the Except Queue at Level 2 or the Approval Queue at Level 3. A second user having a second level of authority may be given access to the Work Queue at Level 1 and the Except Queue at Level 2, but not the Approval Queue at Level 3. A third user having a third (and highest) level of authority may be given access to the Work Queue at Level 1, the Exception Queue at Level 2 and also the Approval Queue at Level 3. Users with access to the Approval Queue at Level 3 are not necessarily given access to the Work Queue at Level 1 or the Exception Queue at Level 2 and the access provided to users may be configured in a wide variety of ways, depending upon a particular implementation. The use of workflow levels provides a flexible and extensive approach that allows for multiple levels of access granularity. FIG. 20C depicts an example workflow 2200 that is the same as workflow 2000 of FIG. 20A, except that approved images are provided to storage, for example storage 168, instead of to EMR system 106.

### IX. IMPLEMENTATION MECHANISMS

Although the flow diagrams of the present application depict a particular set of steps in a particular order, other implementations may use fewer or more steps, in the same or different order, than those depicted in the figures.

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

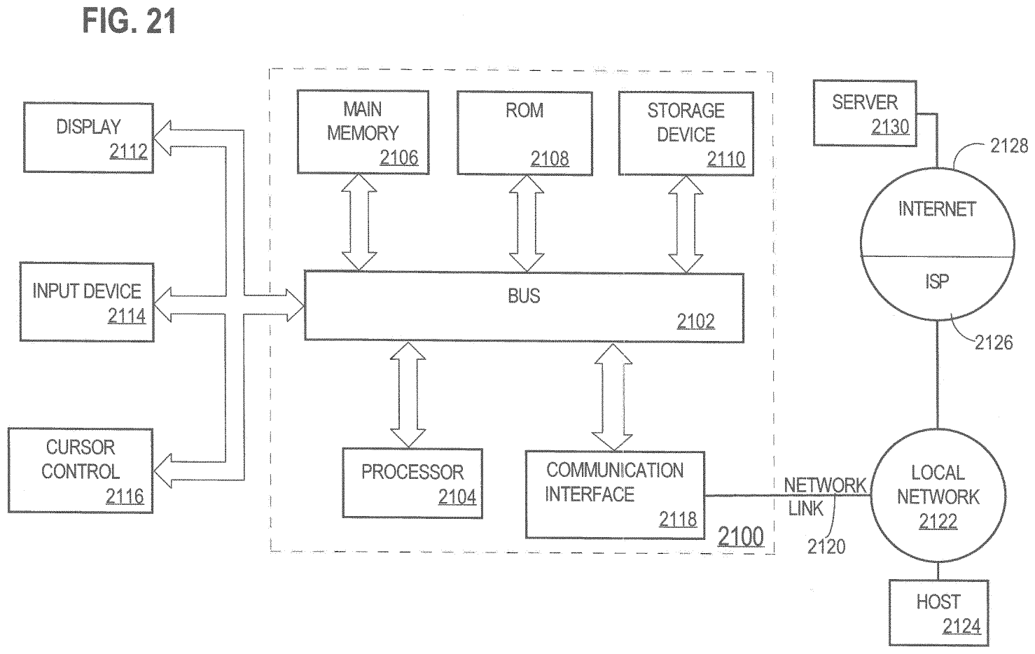

FIG. 21 is a block diagram that depicts an example computer system 2100 upon which embodiments may be implemented. Computer system 2100 includes a bus 2102 or other communication mechanism for communicating information, and a processor 2104 coupled with bus 2102 for processing information. Computer system 2100 also includes a main memory 2106, such as a random access memory (RAM) or other dynamic storage device, coupled to bus 2102 for storing information and instructions to be executed by processor 2104. Main memory 2106 also may be used for storing temporary variables or other intermediate information during execution of instructions to be executed by processor 2104. Computer system 2100 further includes a read only memory (ROM) 2108 or other static storage device coupled to bus 2102 for storing static information and instructions for processor 2104. A storage device 2110, such as a magnetic disk or optical disk, is provided and coupled to bus 2102 for storing information and instructions.

Computer system 2100 may be coupled via bus 2102 to a display 2112, such as a cathode ray tube (CRT), for displaying information to a computer user. Although bus 2102 is illustrated as a single bus, bus 2102 may comprise one or more buses. For example, bus 2102 may include without limitation a control bus by which processor 2104 controls other devices within computer system 2100, an address bus by which processor 2104 specifies memory locations of instructions for execution, or any other type of bus for transferring data or signals between components of computer system 2100.

An input device 2114, including alphanumeric and other keys, is coupled to bus 2102 for communicating information and command selections to processor 2104. Another type of user input device is cursor control 2116, such as a mouse, a trackball, or cursor direction keys for communicating direction information and command selections to processor 2104 and for controlling cursor movement on display 2112. This input device typically has two degrees of freedom in two axes, a first axis (e.g., x) and a second axis (e.g., y), that allows the device to specify positions in a plane.

Computer system 2100 may implement the techniques described herein using customized hard-wired logic, one or more ASICs or FPGAs, firmware and/or program logic or computer software which, in combination with the computer system, causes or programs computer system 2100 to be a special-purpose machine. According to one embodiment, those techniques are performed by computer system 2100 in response to processor 2104 processing instructions stored in main memory 2106. Such instructions may be read into main memory 2106 from another computer-readable medium, such as storage device 2110. Processing of the instructions contained in main memory 2106 by processor 2104 causes performance of the functionality described herein. In alternative embodiments, hard-wired circuitry may be used in place of or in combination with software instructions to implement the embodiments. Thus, embodiments are not limited to any specific combination of hardware circuitry and software.

The term “computer-readable medium” as used herein refers to any medium that participates in providing data that causes a computer to operate in a specific manner. In an embodiment implemented using computer system 2100, various computer-readable media are involved, for example, in providing instructions to processor 2104 for execution. Such a medium may take many forms, including but not limited to, non-volatile media and volatile media. Non-volatile media includes, for example, optical or magnetic disks, such as storage device 2110. Volatile media includes dynamic memory, such as main memory 2106. Common forms of computer-readable media include, without limitation, a floppy disk, a flexible disk, hard disk, magnetic tape, or any other magnetic medium, a CD-ROM, any other optical medium, a RAM, a PROM, and EPROM, a FLASH-EPROM, any other memory chip, memory cartridge or memory stick, or any other medium from which a computer can read.

Various forms of computer-readable media may be involved in storing instructions for processing by processor 2104. For example, the instructions may initially be stored on a storage medium of a remote computer and transmitted to computer system 2100 via one or more communications links. Bus 2102 carries the data to main memory 2106, from which processor 2104 retrieves and processes the instructions. The instructions received by main memory 2106 may optionally be stored on storage device 2110 either before or after processing by processor 2104.

Computer system 2100 also includes a communication interface 2118 coupled to bus 2102. Communication interface 2118 provides a communications coupling to a network link 2120 that is connected to a local network 2122. For example, communication interface 2118 may be a modem to provide a data communication connection to a telephone line. As another example, communication interface 2118 may be a local area network (LAN) card to provide a data communication connection to a compatible LAN. Wireless links may also be implemented. In any such implementation, communication interface 2118 sends and receives electrical, electromagnetic or optical signals that carry digital data streams representing various types of information.

Network link 2120 typically provides data communication through one or more networks to other data devices. For example, network link 2120 may provide a connection through local network 2122 to a host computer 2124 or to data equipment operated by an Internet Service Provider (ISP) 2126. ISP 2126 in turn provides data communication services through the world wide packet data communication network now commonly referred to as the “Internet” 2128. Local network 2122 and Internet 2128 both use electrical, electromagnetic or optical signals that carry digital data streams.

Computer system 2100 can send messages and receive data, including program code, through the network(s), network link 2120 and communication interface 2118. In the Internet example, a server 2130 might transmit a requested code for an application program through Internet 2128, ISP 2126, local network 2122 and communication interface 2118. The received code may be processed by processor 2104 as it is received, and/or stored in storage device 2110, or other non-volatile storage for later execution.

In the foregoing specification, embodiments have been described with reference to numerous specific details that may vary from implementation to implementation. Thus, the sole and exclusive indicator of what is, and is intended by the applicants to be, the invention is the set of claims that issue from this application, in the specific form in which such claims issue, including any subsequent correction. Hence, no limitation, element, property, feature, advantage or attribute that is not expressly recited in a claim should limit the scope of such claim in any way. The specification and drawings are, accordingly, to be regarded in an illustrative rather than a restrictive sense.

### X. OVERVIEW OF A DOCUMENT INTEGRATION PROCESS

In an embodiment, an approach for integrating electronic documents and digital images with data records is presented. The approach is applicable to service provider systems that process vast amounts of documents and images on a daily basis. For example, the approach may be implemented in organizations that provide healthcare services, consulting services, legal services, real estate services, education services communications services, storage services, and the like. Although throughout the disclosure references are made to healthcare services, the disclosure is not limited to applications specific to healthcare. Similarly, although some examples described in the disclosure refer to healthcare services, the examples are not to be viewed as limiting the approach to merely healthcare applications.

A process of integrating electronic documents and digital images implemented in healthcare systems allows a processing of massive amounts of documents, such as patient identification documents, results of laboratory tests, X-rays, faxes and notes from physicians and nurses, disclosures and authorizations obtained from patients, and the like. While some documents and images may be provided as hardcopies, others may be represented in a digital form as text files, image files or combinations of text and image files. The digital files may include electronic documents and digital images.

Electronic documents and digital images may be provided to healthcare facilities from different sources. For example, some of the images may be acquired by scanning devices and transmitted in a digital form as digital images from the scanning devices to one or more processing computers hosted by a healthcare facility. Other digital images may be received via from fax machines, MFP devices, scanners, copiers, and other machines configured to communicate data electronically. Other images may be communicated to the processing computers as data transferred from file servers and other specialized computers.

In an embodiment, processing of the received electronic documents and digital images is performed at a computer server hosted by a service provider. A computer server may be an application server configured as a virtual server on a cloud system, a physical server maintained by a service provider, or any other server accessible to the service provider.

In an embodiment, a processing of the received electronic documents and digital images is automated and performed by one or more management applications executed by one or more computers hosted at a healthcare facility. The management applications may be configured to process the received digital data and associate the received data with for example, corresponding patient records.

An association between a received digital image and a corresponding patient record may be determined based on for example, metadata that is associated with the received digital image. The metadata may be provided from different sources and using a variety of methods, as it is described below.

Based on metadata associated with a received digital image, a determination is made whether the received digital image may be associated with any patient record of a plurality of patient records. Once a corresponding patient record is identified, the received image data may be associated with the identified patient record.

If metadata associated with a received digital image is insufficient to determine a patient record for the received digital image, or if no metadata is associated with the received digital image, then a graphical user interface may be generated to assist a user in providing additional information about the received digital image and to assist in associating the received digital image with some patient record.

In an embodiment, a processing of the received digital data using management applications executed at an application server includes verification and validation of the received data. For example, a received digital image may be automatically analyzed to determine the type of the image. The image may also be automatically validated to determine whether contents of the image is for example useful, and if the image is valid, then the image may be automatically associated with a corresponding patient record or the system may assist a user in associating the image with a corresponding patient record.

In an embodiment, a presented approach at least partially eliminates a need for a time consuming and complex manual processing of received digital images. The approach allows automatically analyzing the received images, verifying and validating the images, determining how the images are to be classified, and assigning the images with corresponding patient records.

A presented approach may be integrated with any electronic medical record (EMR) system. For example, a process of analyzing received digital images and associating the received digital images to patient records may be implemented in one or more application servers that communicate with one or more EMR systems. An EMR system may be updated based on the output generated by the application servers in a pseudo real time, and thus the processing of the received digital images and updating the EMR systems may occur almost simultaneously.

### XI. WORKFLOW OF A DOCUMENT INTEGRATION PROCESS

A document integration process may be implemented in various applications, including healthcare-related applications, service provider applications, and the like. Although the disclosure makes various references to the healthcare-related applications, the provided examples are not viewed as limiting with respect to the applicability of the presented approach.

In an embodiment, a document integration process is performed by executing one or more management applications configured to manage and integrate digital images and electronic documents. The management applications may be configured to process the received images to allow associating the images with for example, the corresponding patient records.

One or more management applications may be executed on one or more application servers hosted at healthcare facilities or available to healthcare service providers. An application server may be a computer server configured as a virtual server on a cloud system, a physical server maintained by a service provider, or any other server accessible to a service provider.

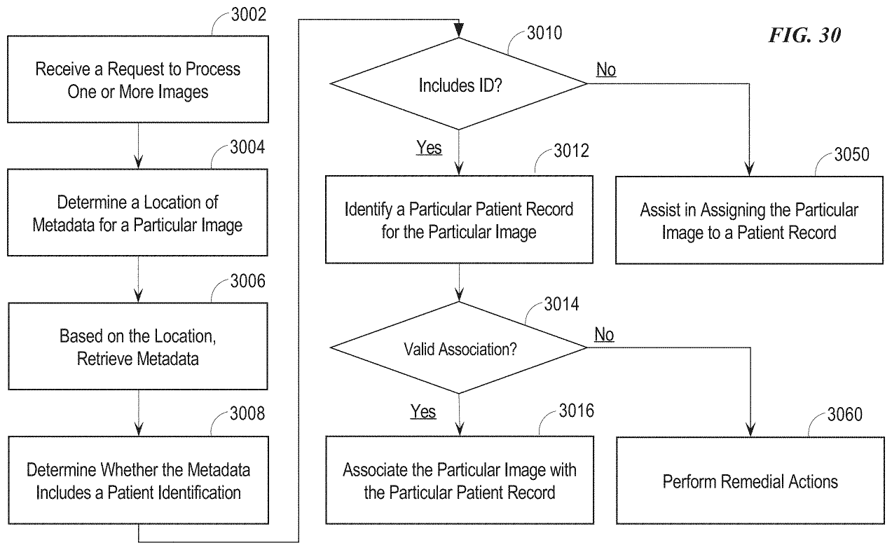

FIG. 30 depicts an example workflow for a document integration process. In step 3002, an image management application receives a request to process one or more images acquired by one or more devices. The images may be provided to a healthcare facility from different sources, such as scanning devices, MFP devices, copiers, and other machines configured to communicate data electronically. For example, an image may be received from an MFP device as a fax. The fax may be transmitted to a file server accessible to a management application executed on an application server hosted by the healthcare provider. Alternatively, the fax may be stored in a data folder that is accessible to the management application, or stored on a storage device accessible to the application server.

In step 3004, a particular image, of the one or more images, is validated to determine whether the particular image may be associated with any patient record. In this step, a location of metadata for the particular image is determined.

Metadata for a particular image may be included in the particular image itself or may be stored on a server and indicated using a reference, such as a uniform resource locator (URL). Various methods for storing metadata are described in the following sections.

If metadata for a particular image is included in the particular image itself, then determining a location of metadata for the particular image may include determining where in the particular image the metadata is present. If metadata for a particular image is stored on a server, then determining a location of metadata for the particular image may include determining a URL pointing to the location.

In step 3006, metadata is retrieved from an identified location. For example, the metadata may be extracted from the particular image or retrieved from a location indicated by an URL.

In step 3008, metadata associated with a particular image is examined to determine whether the metadata includes any form of identification of the patient from a plurality of patients. A patient's identification may include a patient record, a social security number of the patient for whom the image was received, and the like.

If it is determined in step 3010 that metadata associated with a particular image includes some form of identification of the patient, then step 3012 is executed. For example, if metadata associated with a particular image includes a patient medical record number (MRN), then the patient MRN may be used as a patient's identification. Otherwise, step 3050 is executed.

In step 3012, a particular patient record for a particular image is identified from a plurality of patient records. A particular patient record may be identified based on a patient's identification determined in step 3008. For example, a particular patient record may be identified based on a patient's MRN if such is provided in the metadata associated with the particular image.

In step 3014, it is determined whether a particular image may be associated with a particular patient record. To assist in determining whether an association between the particular image and the particular patient record can be made, a GUI may be displayed for a user. The GUI may include a display of the particular image and information about the particular patient record. By examining the displayed information, a user may determine whether associating the particular image with the particular patient record is desirable.

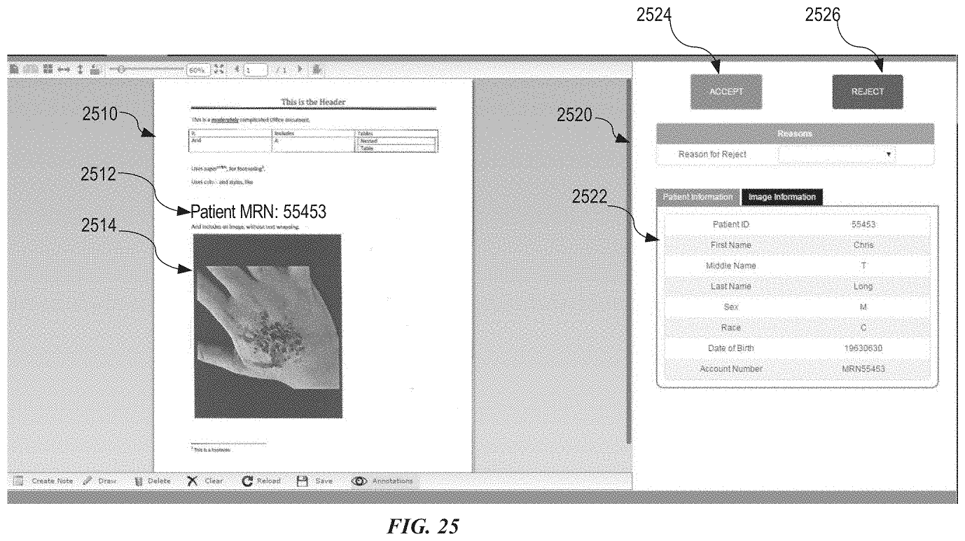

This step is also referred to as validation of a potential association between a particular image and a particular patient record. This step provides a safety measure for assigning received images with patient records. This step is desirable to make sure that the assignments between the images and patient's records are correct. Implementation examples of this step are described in FIG. 25.

If it is determined in step 3014 that an association between a particular image and a particular patient record is desired, the step 3016 is performed. Otherwise, step 3060 is performed.

In step 3016, a particular image is associated with a particular patient record. Associating a particular image with a particular patient record may include displaying a GUI for a user. The GUI may display contents of the particular image, information about the particular patient record, and interactive elements that allow the user to associate the particular image with the particular patient record. This may be implemented using various interactive objects, buttons or icons. Implementation examples of associating a particular image with a particular patient record are described in FIG. 25.

Associating a particular image with a particular patient record may include storing the particular image in association with the particular patient record and making the particular image available upon accessing the particular patient record. For example, if a particular image is a photograph depicting a wound on a patient's arm and the particular image is associated with a particular patient record, then, once the particular image is associated with the particular patient record, a physician or a nurse who has access to the particular patient record may be able to download and display the particular image depicting the wound.

The GUI may also allow the user to reject a suggested association between the particular image and the particular patient record. For example, the GUI may include a display of the contents of the particular image, information about the particular patient record, and interactive elements that allow a user to reject a suggested association between the particular image and the particular patient record.

In addition, if it is determined in step 3014 that an association between a particular image and a particular patient record is undesired (or would be incorrect), then in step 3060, one or more remedial actions are performed. For example, a GUI may be displayed for a user to present the particular image and a portal for reviewing patient records, and to assist the user in finding another patient record with which the particular image may be associated. Alternatively, a GUI may be displayed for a user to allow generating a new patient record and assigning the particular image with the new patient record. Furthermore, a GUI may be displayed to allow the user to discard the particular image if the user determines that the particular image cannot be assigned with any patient record.

If it is determined in step 3010 that metadata associated with a particular image does not include or otherwise provide sufficient information about any patient record, then, in step 3050, a GUI is displayed to assist a user in determining whether the received particular image may be associated with any patient record. The GUI may display the contents of the particular image and a search utility that may assist a user in searching the patient records. Using the GUI, the user may inspect the contents of the received fax and the associated metadata, and if the image and the metadata are approved as valid or legitimate, then the user may search the patient records to determine a corresponding patient record to which the image may be assigned. If a corresponding patient record for the received fax is found, then the user may manually assign the received fax with the corresponding patient record. If the record is not found, then the user may try to examine the metadata associated with the received image and try to determine whether the received image can in any way be associated with any of the patient records.

If an association between a received image and a corresponding patient record is verified by an authorized person or a manager, then the association may be integrated with for example, an EMR system. This may be accomplished by providing for example, an indication of the association to the EMR system that the received image has been associated with the particular patient record. Furthermore, the received image and the indication of the association may be transmitted to an EMR system of a healthcare service provider.

### XII. EXAMPLE DOCUMENT INTEGRATION PROCESS

Processing of digital images that may take place at a healthcare provider facility may be illustrated using the following example. Upon receiving, at an MFP device, a fax communication (also referred herein to as a fax), the MFP device may initiate a filing workflow for processing of the received fax. The filing workflow may include transmitting the received fax to a management application executed on an application server hosted by the healthcare provider. The transmission may also include transmitting the received fax to a data folder that is accessible to the management application executed on the application server, or storing the received fax on a storage device accessible to the application server and sending, to the management application, an URL identifying the location at which the received fax has been stored.

A received fax may include metadata or an indication where the metadata associated with the received fax may be found. The metadata may be extracted or otherwise retrieved and used to provide some identification for the received fax. The identification may include a patient record, a social security number of the patient for whom the fax communication was received, and the like. The metadata and any other related information may be determined by processing the received fax or the retrieved metadata using an optical character recognition (OCR) reader, a quick response (QR) code reader, and the like.

A received fax and/or associated metadata may be processed according to a data integration workflow specific to the processing of fax communications. The data integration workflow may include validating of the received fax, inspecting of the received fax and/or the associated metadata, modifying contents of the metadata, and the like.

A data integration workflow may be implemented as an interactive process in which corresponding contents are displayed for a user in a GUI, and the user may inspect the contents and perform one or more actions with respect to the contents. For example, a received fax and the associated metadata may be verified or inspected by an authorized person or a manager, and if the fax and the metadata are approved as valid or legitimate, then a management application may be invoked to determine a corresponding patient record to which the received fax may be assigned. If a corresponding patient record for the received fax is found, then the indication of the association, the fax and/or metadata may be transmitted to an EMR system that a healthcare provider hosts or utilizes.

In an embodiment, an association between a received fax and a corresponding patient record may be verified by an authorized person or a manager. If the association is approved, then an indication of the association, the received fax and/or the associated metadata may be transmitted to an EMR system of a healthcare service provider.

An association between a received fax and a corresponding patient record may be represented by including an indicator of the corresponding patient record in the metadata associated with the received fax. An indicator may be an alphanumerical string representing a patient identifier, a patient social security number, a patient record identifier, and the like. Alternatively, an indicator may include an URL indicating a location at which the information about the corresponding patient record is stored.

However, if a corresponding patient record cannot be determined based on the received fax document and/or the associated metadata, then a GUI may be displayed for a user or an authorized person to assist the user to determine the corresponding patient record. The GUI may be configured to assist the user in navigating through a library of patient records and performing searches of patient records. The GUI may also be configured to allow the user to provide additional information about the received fax, modify the contents of the received fax and/or modify the associated metadata, and the like.

### XIII. ARRANGEMENTS FOR ACQUIRING AND MANAGING DIGITAL IMAGES

A document integration process may be implemented using a variety of hardware-software-based arrangements. Examples of the arrangements may include two groups of arrangements: the arrangements in which the digital images are received from MFP devices and the arrangements in which the digital images are received from various servers, digital cameras, and the like.

Each of the two groups may be further divided into respective subgroups. For example, the arrangements in which the images are received from an MFP device may include the arrangements in which the images are transmitted from the MFP device to an FTP based server using for example, an FTP-based data transfer, and the arrangements in which the images are transmitted from the MFP device to corresponding data folders or data directories.

Similarly, the arrangements in which the images are received from a hardware-implemented server may include the arrangements in which the images are transmitted from the server to an FTP-server using for example, an FTP-based data transfer, and the arrangements in which the images are transmitted from the server to corresponding data folders or data directories.

### XIV. EXAMPLE ARRANGEMENTS FOR ACQUIRING AND MANAGING DIGITAL IMAGES RECEIVED FROM MULTIFUNCTION PERIPHERAL DEVICES

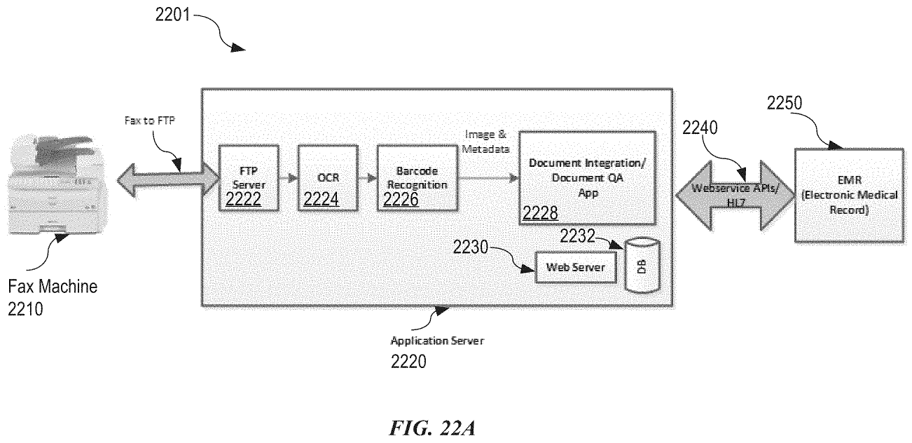

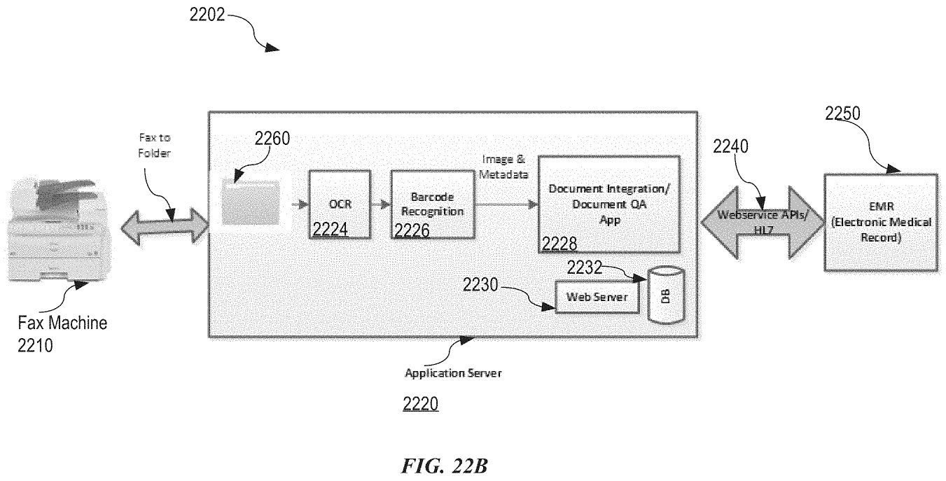

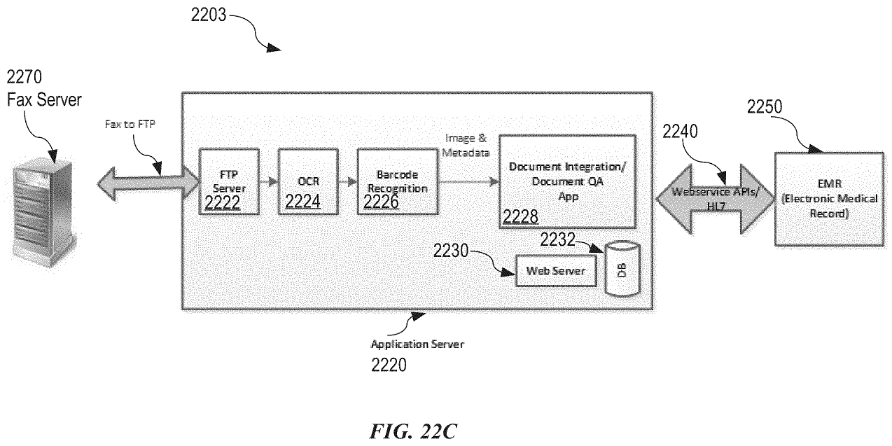

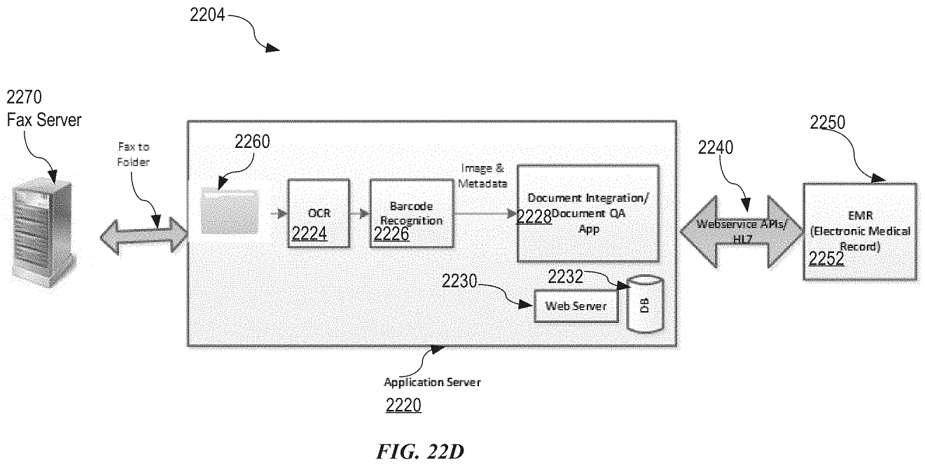

In an embodiment, a document integration process is implemented in arrangements in which the digital images are received from MFP devices. Examples of such arrangements may include the arrangements in which the images are transmitted from the MFP device to an FTP server, and the arrangements in which the images are transmitted from the MFP device and stored in corresponding data folders or data directories. An example of the arrangements wherein the images are transmitted to an FTP server is described in FIG. 22A. An example of the arrangements where the images are stored in corresponding data folders or data directories is described in FIG. 22B.

A. File-Transfer-Based Arrangements

In an embodiment, a process of acquiring and managing of digital images is implemented in arrangements in which the digital images are received from one or more MFP devices and transmitted from the MFP devices to FTP servers via FTP-based communications connections.

FIG. 22A is a block diagram that depicts an arrangement 2201 for acquiring and managing digital images received from an MFP device 2210 and transmitted to a file server, such as for example, an FTP server 2222. In arrangement 2201, MFP device 2210 is communicatively coupled with an application server 2220, which is communicatively coupled with one or more EMR systems 2250.

In arrangement 2201, MFP device 2210 is any type of a MFP configured to receive and process any type of electronic data. For example, MFP device 2210 may be configured to receive electronic emails, receive fax communications, generate digital images by photocopy hardcopies of documents, generate digital images by scanning hardcopies of documents, generate digital images by printing hardcopies documents into a PDF data files, and the like. Depending on the implementation, MFP device 2210 may be configured to perform all the above listed functions, or may be configured to perform a subset of the above listed functions. For example, in some implementations, MFP device 2210 may be configured to receive and transmit faxes, and in such implementations, MFP device 2210 may be viewed as a fax machine. In other implementations, MFP device 2210 may be configured to scan hardcopies of documents, and in such implementations, MFP device 2210 may be viewed as a scanner. Other configuration of MFP device 2210 may also be implemented.

Application server 2220 may include one or more components that are configured to receive and process digital image data. Application server 2220 may include for example, an FTP server 2222, an optical character recognition (OCR) component 2224, a bar recognition component 2226, and a document integration and processing component 2228. Application server 2220 may also include one or more web servers 2230 and/or may communicate with one or more external web servers (not depicted in FIG. 22A). Furthermore, application server 2220 may include one or more data storages, such as for example, a database 2232.

FTP server 2222 is a computer system configured to receive and transmit data communications in compliance with the FTP network protocol over a computer network. FTP server 2222 may be configured to execute one or more FTP client applications and one or more FTP server applications that facilitate data transmission. In alternative embodiments, server 2222 may be configured to transmit data communications in compliance with other data communications protocols, such as the transmission control protocol (TCP), the Internet protocol (IP), the TCP/IP, and the like.

OCR component 2224 is a component configured to perform an electronic conversions of images that are typed, handwritten or printed into machine-encoded texts. In healthcare-related implementations, OCR component 2224 may be configured to process digital images containing medical laboratory test results, medical receipts, medical invoices, billing documents, and the like into machine-encoded texts.

Bar recognition component 2226 is a component configured to perform an electronic conversion of images depicting barcodes having optical machine-readable representation of encoded data. In healthcare-related implementations, bar recognition component 2226 may be configured to process barcodes representing encoded identifiers. The encoded identifiers may include the identifiers of patient identifications, identifiers of medical tests, identifiers of medical records, identifiers of hospital departments, identifiers of documents, and the like.

In an embodiment, bar recognition component 2226 is configured to decode one or more types of barcodes that include encoded representations of data. The barcodes may include quick response (QR) codes, radio-frequency identification (RFID) codes, and the like.

Document integration and processing component 2228 is configured to determine whether a received digital image may be association with any patient record. Component 2228 may also be configured to associate the received image with a particular patient record if such an association is possible. Furthermore, component 2228 may be configured to assist a user of application server 2220 in reviewing the received digital image, metadata associated with the digital image, patient records, and modify such if it is necessary.

Web server 2230 is any type of a server configured to support the acquiring and managing of digital images. For example, web server 2230 may be used to store metadata associated with the digital images. Web server 2230 may also be used to store copies of patient records, and the like. Web server 2230 may be implemented as part of application server 2220, as it is depicted in FIG. 22C. In other implementations, web server 2230 may be an external server that is communicatively coupled with application server 2220.

In an embodiment, MFP device 2210 transmits a fax, or other digital communications, to FTP server 2222 via a communications connection configured to transfer electronic data in compliance with a communications protocol, such as the FTP.

Upon receiving from MFP device 2210 a fax, or other digital communications, FTP server 2222 pre-processes the received digital image. For example, FTP server 2222 may determine whether the digital image is to be transmitted to OCR component 2224 and/or barcode recognition component 2226 for further processing.

If a received digital image is transmitted to OCR component 2224, then upon receiving the digital image, OCR component 2224 may convert the image into machine-encoded text. In healthcare-related implementations, OCR component 2224 may be configured to process a digital image containing one or more of a patient identifier, a medical laboratory test result, a medical receipt, a medical invoice, billing document, and the like into machine-encoded text. The resulting encoded text may include alphanumerical strings corresponding to one or more of a patient identifier, a medical laboratory test result, a medical receipt, a medical invoice, billing document, and the like. The resulting text may be further processed by barcode recognition component 2226. The resulting alphanumerical strings may be referred to as metadata. The metadata may be stored in association with the received image, or may be stored at a location identified using a URL pointing to a location on webserver 2230 or database 2232.

If a received digital image is transmitted to barcode recognition component 2226, then upon receiving the digital data, barcode recognition component 2226 may convert one or more barcodes depicted in the digital image into one or more alphanumerical strings. In healthcare-related implementations, bar recognition component 2226 may be configured to process barcodes representing encoded identifiers, such as identifiers of patient identifications, identifiers of medical tests, identifiers of medical records, identifiers of hospital departments, identifiers of documents, and the like. The resulting alphanumerical strings may be referred to as metadata. The metadata may be stored in association with the received image, or may be stored at a location identified using a URL pointing to a location on webserver 2230 or database 2232.

A received fax and associated metadata, or an indication where the metadata associated with the received fax may be found, may be transmitted to document integration and processing component 2228, also referred to as a component 2228.

Component 2228 may receive or otherwise retrieve the digital image and the associated metadata and based on the received information, determine if the received digital image may be associated with any patient record. For example, component 2228 may try to determine whether the digital image or the metadata includes a patient record, a social security number of the patient for whom the fax communication was received, and the like.

Component 2228 may also provide tools that a user may use to validate, inspect, and/or modify contents of the received fax, the associated metadata, and/or the association between the received image and a particular patient record. Component 2228 may also be configured to generate a GUI for a user, and display the contents of the image, the metadata and the associations in the GUI. The GUI may allow the user to inspect the contents and perform one or more actions on the contents. For example, a user may validate and inspect the received fax, the associated metadata and the association between the received fax and a corresponding patient record.

If a corresponding patient record cannot be determined for a received fax document and based on the associated metadata, then component 2228 may generate and cause displaying for a user a GUI configured to assist the user in navigating through a library of patient records and perform searches of patient records. The GUI may also be configured to allow the user to provide additional information about the received fax, modify the contents of the received fax and/or modify the associated metadata, and the like.

Component 2228 may also be configured to access one or more web services and/or one or more applications that are configured to communicate with one or more EMR systems 2250. For example, in a healthcare service provider implementations, application server 2220 may be configured to communicate the results of the document integration process to one or more EMR systems 2250 using a web service and/or specialized application program interface 2240. The web service or the specialized application program interface 2240 may be compatible with any of the international standards know in the healthcare community to transfer clinical and administrative data between software applications used by various healthcare providers. Examples of such standard may include a health level-7 (HL7) standard, which implements the seventh layer of the Open System Interconnect (OSI) model and which is typically used by healthcare providers such as hospitals, medical clinics, and the like.

In an embodiment, processing of the received digital data as depicted in FIG. 22A allows automatically analyzing the received images, automatically determining how the received digital image is to be classified, and automatically assigning the received digital image with a corresponding patient record. Furthermore, the presented approach may be integrated with any EMR system and allow an automatic update of one or more EMR systems.

B. Folder-Based Arrangements

In an embodiment, a process of acquiring and managing of digital images is implemented in arrangements in which the digital images are received from one or more MFP devices and stored in a data folder or a file folder.

FIG. 22B is a block diagram that depicts an arrangement 2202 for acquiring and managing digital images received from an MFP device 2210 and stored in a data folder 2260.

In arrangement 2202, MFP device 2210 is any type of a MFP configured to receive and process any type of electronic data. Examples of MFP devices 2210 are described in FIG. 22A.

Application server 2220 may include one or more components such as for example, an optical character recognition (OCR) component 2224, a bar recognition component 2226, a document integration and processing component 2228, one or more web servers 2230, and one or more data storages, such as for example, a database 2232. Examples of the above listed devices are described in FIG. 22A.

Application server 2220 may also include one or more file servers configured to provide electronic file folders, such as an electronic file folder 2260.

In an embodiment, electronic file folder 2260 is an electronic directory virtually or otherwise organized on an electronic disk. Electronic file folder 2260 may be configured to store data items such as digital images, data files, text files, and the like. Electronic file folder 2260 is also referred herein to as a folder 2260.

In an embodiment, MFP device 2210 receives a fax or other digital communications. Upon receiving the fax, MFP device 2210 may establish a communications connection with application server 2220, access folder 2260 maintained by application server 2220, and store the received fax in folder 2260. Folder 2260 may be identified by a folder name, a URL pointing to the folder 2260, or any other identifier. For example, MFP device 2210 may be provided with a name of folder 2260, access folder 2260 having the provided name, and initiate an FTP-based transfer of the fax to the folder 2260.

Once the received fax is stored in folder 2260, document integration and processing component 2228 executed on application server 2220 pre-processes the received fax. For example, component 2228 may determine whether the digital image is to be transmitted to OCR component 2224 and/or barcode recognition component 2226 for further processing. Processing by OCR component 2224 and barcode recognition component 2226 is described in FIG. 22A.

Results generated by OCR component 2224 and barcode recognition component 2226 are referred to herein as alphanumerical strings or metadata. The metadata may be stored in association with the received image, or may be stored at a location identified using a URL pointing to a location on webserver 2230 or database 2232.

Further processing of the received fax and associated metadata may be performed by component 2228, and is described in detail in FIG. 22A.

In an embodiment, processing of the received digital data described in FIG. 22B allows automatically analyzing the received images and stored in a file folder maintained by application server 2220. Furthermore, the processing described in FIG. 22B allows automatically determining how the received digital image is to be classified, and automatically assigning the received digital image with a corresponding patient record and integrate the results with one or more EMR systems.

### XV. EXAMPLE ARRANGEMENTS FOR ACQUIRING AND MANAGING DIGITAL IMAGES RECEIVED FROM SERVERS

In an embodiment, presented document integration processes are implemented in arrangements in which the digital images are received from data servers. Examples of such arrangements may include the arrangements in which the images are transmitted from a server to an FTP server, and the arrangements in which the images are transmitted from a server and stored in corresponding data folders or data directories. An example of the arrangements wherein the images are transmitted to an FTP server is described in FIG. 22C. An example of the arrangements where the images are stored in corresponding data folders or data directories is described in FIG. 22D.

A. File-Transfer-Based Arrangements

In an embodiment, a process of acquiring and managing of digital images is implemented in arrangements in which the digital images are received from a server and transmitted from the server to FTP servers via FTP-based communications connections.

FIG. 22C is a block diagram that depicts an arrangement 2203 for acquiring and managing digital images received from any type of server, including a fax server 2270, and transmitted to a file server, such as for example, an FTP server 2222. In arrangement 2203, fax server 2270 is communicatively coupled with an application server 2220, which is communicatively coupled with one or more EMR systems 2250.

Application server 2220 and some of its components are described in detail in FIG. 22A.

In an embodiment, fax server 2270 transmits a fax, or other digital communications, to FTP server 2222 via a communications connection configured to transfer electronic data in compliance with a communications protocol, such as the FTP.

Upon receiving a fax, or other digital communications, FTP server 2222 pre-processes the received digital image. For example, FTP server 2222 may determine whether the digital image is to be transmitted to OCR component 2224 and/or barcode recognition component 2226 for further processing. The OCR processing and the barcode processing are described in detail in FIG. 22A.

Results generated by OCR component 2224 and barcode recognition component 2226 are referred to herein as alphanumerical strings or metadata. The metadata may be stored in association with the received image, or may be stored at a location identified using a URL pointing to a location on webserver 2230 or database 2232. Further processing of the received fax and associated metadata may be performed by component 2228, as described in detail in FIG. 22A.

In an embodiment, a processing of the received digital data as described in FIG. 22C allows automatically analyzing the received images transmitted to FTP server 2222 maintained by application server 2220. Furthermore, the processing described in FIG. 22C allows automatically determining how the received digital image is to be classified, and automatically assigning the received digital image with a corresponding patient record and integrate the results with one or more EMR systems.

B. Folder-based Arrangements

In an embodiment, a process of acquiring and managing of digital images is implemented in arrangements in which the digital images are received from a computer server and stored in a data folder or a file folder.

FIG. 22D is a block diagram that depicts an arrangement 2204 for acquiring and managing digital images received from any type of server, including a fax server 2270, and stored in any type of electronic data folder, including a data folder 2260.

Application server 2220 and its components are described in detail in FIG. 22A.

Application server 2220 may include one or more file servers configured to provide electronic file folders, such as an electronic file folder 2260.

In an embodiment, electronic file folder 2260 is an electronic directory virtually or physically organized on an electronic disk. Electronic file folder 2260 may be configured to store data items such as digital images, data files, text files, and the like. Electronic file folder 2260 is also referred herein to as a folder 2260.

In an embodiment, fax server 2270 receives a fax or other digital communications. Upon receiving the fax, fax server 2270 may establish a communications connection with application server 2220, access folder 2260 maintained by application server 2220, and store the received fax in folder 2260. Folder 2260 may be identified by a folder name, a URL pointing to the folder 2260, or any other identifier. Various method of accessing folder 2260 and storing the received fax in folder 2260 are described in FIG. 22C.

Once the received fax is stored in folder 2260, document integration and processing component 2228 executed on application server 2220 pre-processes the received fax. For example, component 2228 may determine whether the digital image is to be transmitted to OCR component 2224 and/or barcode recognition component 2226 for further processing. Processing by OCR component 2224 and barcode recognition component 2226 is described in FIG. 22A.

Results generated by OCR component 2224 and barcode recognition component 2226 are referred to herein as alphanumerical strings or metadata. The metadata may be stored in association with the received image, or may be stored at a location identified using a URL pointing to a location on webserver 2230 or database 2232. Further processing of the received fax and associated metadata may be performed by component 2228, as described in detail in FIG. 22A.

In an embodiment, a processing of the received digital data as described in FIG. 22D allows automatically analyzing the received images and stored in a file folder maintained by application server 2220, automatically determining how the received digital image is to be classified, and automatically assigning the received digital image with a corresponding patient record and integrate the results with one or more EMR systems.

### XVI. METADATA

Metadata may be any type of data that describes other data. For example, metadata associated with a digital image may be any type of data that describes the digital image, contents of the digital image, and the like.

Metadata may include a summary of the basic information about other data and thus allow the processing of the other data easily or conveniently. For example, metadata associated with a fax image or included in a fax image may provide context for the fax. The context may include information specific to the fax, such as a summary of the contents of the fax, a name of a person who sent the fax, a date when the fax was sent, and the like.

In healthcare-related implementations, metadata included in or associated with a digital image may provide a description of the contents represented in the digital image. For example, if a digital image represents results of a laboratory test, then metadata included in the digital image or associated with the digital image may include a medical record number (MRN) or a name of the patient for whom the test was performed, a type of the test, a name of the healthcare provider that provides an insurance coverage to the patient, a name of the department that ordered the test, and the like.

Metadata may be represented in a variety of forms. For example, metadata may be represented as barcodes, QR codes, RFID codes, alphanumeric strings, pictures, logos, and other forms of encoded information.

Metadata may be retrieved from one or more sources. Some metadata may be included in a digital image itself. In other cases, metadata may be attached to a digital image or stored separately from a digital image. This may be implemented using URLs pointing to the locations of the metadata is stored. This also may be implemented using names of electronic folders at which the metadata is stored. The URLs and the names of the electronic folders may be either included in the digital images, or stored at known locations on a server.

A. Example Metadata Represented as Barcodes and Alphanumerical Strings

In an embodiment, a digital image includes metadata encoded and represented using different techniques and approaches. For example, a digital image may include some metadata that is encoded as barcodes and some other metadata that is represented as alphanumeric strings. Furthermore, one digital image may include some metadata that is encoded as RFID barcodes, other metadata that is encoded as QR codes, and yet other metadata that is represented using alphanumerical strings. Other combination of different representations of metadata may also be implemented.

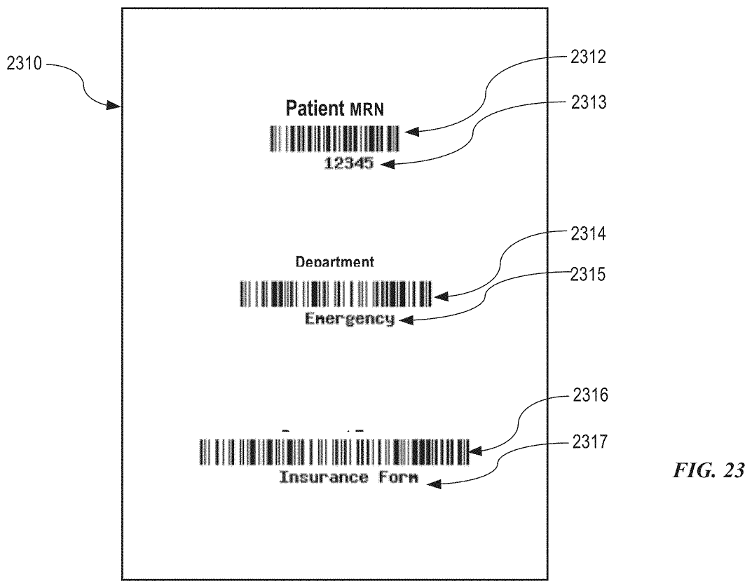

FIG. 23 is an example digital image 2310 that includes metadata represented as barcodes and metadata represented as alphanumeric strings. Digital image 2310 may be any type of digital image provided to an application server responsible for integrating the image with another data system. For example, digital image 2310 may be a cover page of a fax that also includes test results of tests performed for a particular patient. Digital image 2310 may also be a cover page of a fax that also includes a copy of the insurance identification card that was issued to a particular patient. Digital image 2310 may also be any other electronic document or a digital image provided to a healthcare service provider.

In the depicted example, digital image 2310 includes metadata represented as barcodes and metadata represented as alphanumerical string. The metadata represented using barcodes include a patient MRN barcode 2312, a department barcode 2314, and a document type barcode 2316. In the depicted example, patient MRN barcode 2312 is an RFID barcode that encodes a particular medical registration number of a patient, department barcode 2314 is an RFID barcode that encodes the name of the emergency department, and document type barcode 2316 is an RFID barcode that encodes the type of the insurance form. Other types of encoding the metadata may also be used.

Metadata represented in FIG. 23 using alphanumeric strings include a patient MRN number 2313, a department name “Emergency” 2315, and an “Insurance Form” document type 2317. The examples shown in FIG. 23 are provided to merely illustrate non-limiting examples of various types of metadata that may be useful in healthcare-related implementations.

B. Example Metadata Represented as Alphanumerical Strings

In an embodiment, a digital image includes metadata represented as alphanumeric strings. Other representations of metadata may also be implemented.

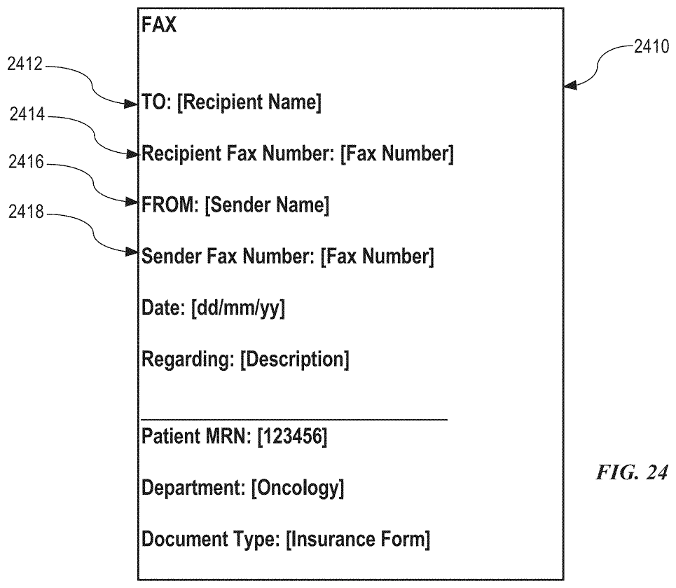

FIG. 24 is an example of a fax cover sheet 2410 containing metadata represented as alphanumeric strings. Fax cover sheet 2410 may be a cover page of a fax that also includes test results of tests performed for a particular patient.

In the depicted example, fax cover sheet 2410 includes metadata represented as alphanumerical string. The examples of the metadata included in fax cover sheet 2410 include a fax recipient name 2412, a recipient fax number 2414, a fax sender name 2416, and a sender fax number 2416, and others. The other examples may include a date when the fax was sent, a description of the contents of the fax, a patient MRN number, a name of the department that ordered the test, a type of the included document, and the like. The examples shown in FIG. 24 are provided to merely illustrate non-limiting examples of various types of metadata that may be useful in healthcare-related implementations.

### XVII. VERIFICATION AND VALIDATION OF AN IMAGE INTEGRATION

In an embodiment, an automatic process of integrating digital images with patient records maintained by EMR systems is subjected to verification and validation. Verification and validation are often independent procedures that are used together to check whether the outcome generated by the process meet the requirements and specification set forth in the process and whether the outcome generate by the process fulfills the set forth purpose.

Verification is usually intended to check whether the outcome of the process meets a set of design specification, while validation is usually intended to check whether the outcome of the process meets the needs of the user. For example, if executing a process of integrating a particular image with a particular patient record results in generating a particular association between the particular image and the particular patient record, then the verification allows determining whether the particular association meets the requirements for generating associations, while validation allows determining whether integrating the particular association into an EMR system would be useful to patients and medical personnel.

In an embodiment, a process of integrating an image with patient records includes determining whether a patient record may be found for the image, whether the image may be associated with the patient record, whether an association between the image and the patient record is valid and whether the association is to be included in an EMR system. The above steps are illustrated in FIG. 25-29 described below.

A. Association Validation

In an embodiment, a process of integrating an image with patient records includes verifying whether a patient record may be found for the image and if so, validating whether an association between the image and the patient record is to be included in an EMR system.

FIG. 25 depicts an example GUI that allows a user to review an image and metadata and determine whether the image is to be associated with a patient record. The GUI depicted in FIG. 25 is one of many examples of the GUI configured to allow the user to review an image, metadata and a suggested association between the image and a patient record.

In an embodiment, to assist a user in determining whether an association between a particular image and a particular patient record can be made, a GUI is displayed for the user. The GUI may include a first display portion 2510, in which a content of the particular image is displayed, and a second display portion 2520, in which information about a particular patient record is displayed. By examining the displayed information, a user may determine whether associating the particular image with the particular patient record is desirable or valid.

In the depicted example, first display portion 2510 shows a display of metadata 2512 and an image 2514. Metadata 2512 is associated with image 2514 showing a picture of a hand with a wound. Metadata 2512 includes a MRN number of a patient, and indicates that image 2514 is to be associated with a patient record of the patient whose MRN number is 55453.

Metadata 2512 may be used to search patient records maintained by an EMR system. In the depicted example, metadata 2522 includes a MRN number 55453. That number may be used to generate a search query, and the search query containing the number 55453 may be used to retrieve a patient record that is associated with the MRN number 55453. Information 2522 of the patient record associated with the MRN number 55453 may be displayed in second display portion 2520.

Second display portion 2520 shows a display of a patient record 2522 and an interactive accept button 2524 and an interactive reject button 2526. Patient record 2522 may include various information about the patient. For example, patient record 2522 may include a MRN number of the patient, a first name of the patient, a middle name of the patient, a last name of the patient, a date of birth, and the like.

Interactive accept button 2524 may be used to accept a suggested association between image 2514 displayed in first display portion 2510 and a patient record whose information 2522 is displayed in second display portion 2520. For example, a user may inspect the image 2514 displayed in first display portion 2510 and information 2522 displayed in second display portion 2520, and determine whether image 2514 is to be associated with patient record 2522. If the user determines that these two are to be associated, then the user may select interactive accept button 2524 to cause associating image 2514 with patient record 2522, and integrating the association with an EMR system.

However, if a user determines that image 2514 is not be associated with patient record 2522, then the user may select interactive reject button 2526 to reject an association between image 2514 and patient record 2522.

B. Image Validation

In an embodiment, a process of integrating an image with patient records includes verifying whether a patient record may be found for the image and if not, determining whether the image is to be accepted or discarded.

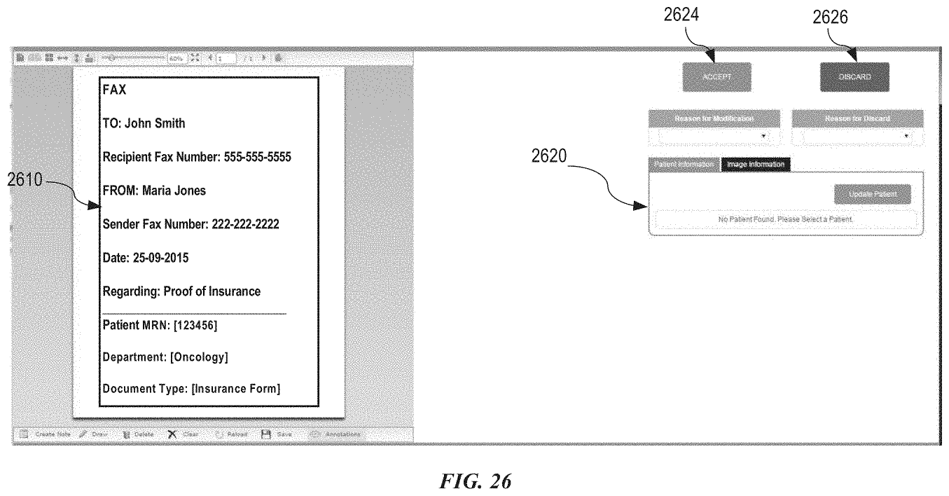

FIG. 26 depicts an example GUI that allows a user to review an image and metadata and determine whether the image is to be accepted. The GUI depicted in FIG. 26 is one of many examples of the GUI configured to allow the user to review an image, metadata and determine if the image is to be accepted or discarded.

In an embodiment, to assist a user in determining whether an image is to be accepted or discarded, a GUI is displayed for the user. The GUI may include a first display portion 2610, in which a content of the particular image is displayed, and a second display portion 2620, in which information indicating whether a particular patient record for the image has been found. By examining the displayed information, a user may determine whether the image is to be accepted or discarded.

In the depicted example, first display portion 2610 shows a fax. The fax includes various types of information, including a name of the patient, a MRN number 123456 of a patient, various phone numbers, a date, and the like. The depicted MRN number 123456 indicates that the fax is to be associated with a patient record of the patient whose MRN number is 123456. The MRN number 123456 may be used to generate a search query, and the search query containing the number 123456 may be used to retrieve a patient record that is associated with the MRN number 123456. However, if the search executed on the patient records has not returned any patient record that an indication of that may be displayed in second display portion 2620.

In the example depicted in FIG. 26, second display portion 2620 shows a display indicating that no patient record was found based on metadata included in the fax displayed in first display portion 2610. Second display portion 2620 also displays an interactive accept button 2624 and an interactive discard button 2626.

Interactive accept button 2624 may be used to accept the fax displayed in first display portion 2610 even if a corresponding patient record cannot be found. For example, a user may inspect the fax displayed in first display portion 2610, and determine whether the fax is to be accepted for further processing or discarded. If the user determines that the fax is to be accepted for further processing, then the user may select interactive accept button 2624 to cause further processing of the fax. The further processing may for example, allow the user to create a new patient record, correct metadata included in the fax and re-execute the process of integrating the fax with patient records.

However, if a user determines that the fax displayed in first display portion 2610 is not be further processed, then the user may select interactive discard button 2626 to discard the fax. In some implementation, that may cause displaying another GUI to allow the user to double check whether the fax is indeed to be discarded.

The above example may be expanded to any type of digital images and electronic documents. For example, a GUI may display in first display portion a picture of a wound and a URL pointing to metadata associated with the picture. If an association between the picture and any patient record cannot be determined, then a user may either select interactive accept button 2624 to accept the picture for further processing, or select interactive discard button 2626 to discard the picture.

C. Patient Record Verification

In an embodiment, a process of integrating an image with patient records includes verifying whether a patient record may be found for the image.

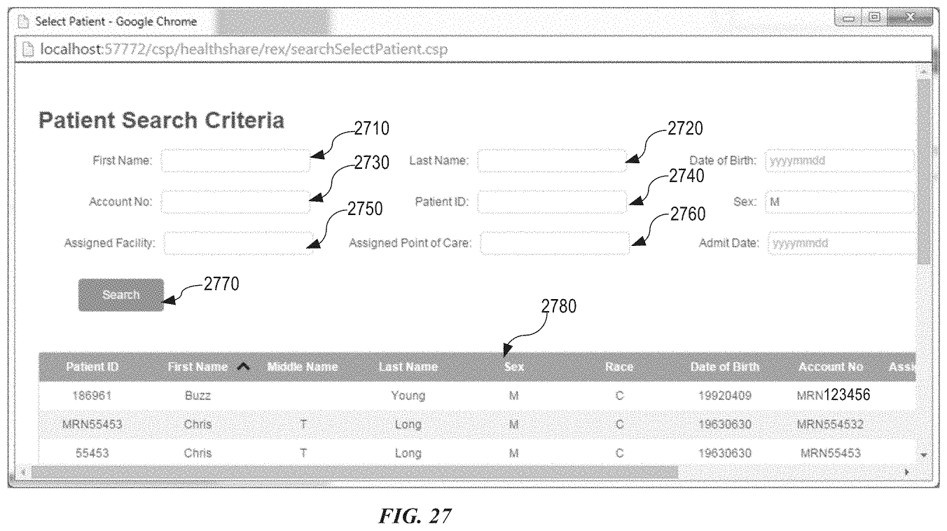

FIG. 27 depicts an example GUI that allows a user to search patient records to determine a patient record for an image. The GUI depicted in FIG. 27 is one of many examples of the GUI configured to allow the user to search patient records maintained by an EMR system.

In an embodiment, to assist a user in determining a patient record that could be associated with a received image, a GUI is displayed for the user. The GUI may include a plurality of text boxes for entering search keywords and phrases for a search query to be executed on a body of the patient records.

In the depicted example, a GUI includes a first name box 2710 to which a user may enter a first name of the patient whose record is sought. The GUI may also include a last name box 2720 to which a user may enter a last name of the patient whose record is sought. Furthermore, the GUI may include other text boxes, such as an account number box 2730, a patient identification box 2740, an assigned facility box 2750, an assigned point of care 2760, and the like.

A user may fill out one or more of the boxes displayed in the GUI depicted in FIG. 27. If at least one box is filled with a keyword, a user may select an interactive search button 2770 to initiate execution of a search query containing the keywords entered by the user. In response to executing the search query on a body of the patient records, one or more matching search results may be returned to the user.

In the example depicted in FIG. 27, in response to a search query, a matching search results 2780 are displayed in a GUI. The matching results include one or more patient records that match the keywords included in the search query. A user may browse the search results and select a particular patient record to be associated with a received image.

This may be illustrated using the following example. Referring again to FIG. 26, metadata included in the depicted fax indicates that a patient MRN is 123456; however, a patient record corresponding to “123456” could not be found in a database of the patient records. If a user selected interactive accept button 2624, the user could receive a display of the GUI depicted in FIG. 27. The user could enter into the GUI the search criteria for searching the database of the patient records. For example, the user could enter some information about the patients that might have the MRN number “123456.” In response to providing the search criteria, the user could receive search results 2780 depicted in FIG. 27. The user may notice that MRN numbers listed in the database of the patient records include a prefix “MRN.” That could help the user to identify a patient whose MRN number is “MRN123456.”

D. Image Validation When A Patient Record Has Been Verified

In an embodiment, a process of integrating an image with patient records includes validating the image when a patient record for the image was verified.

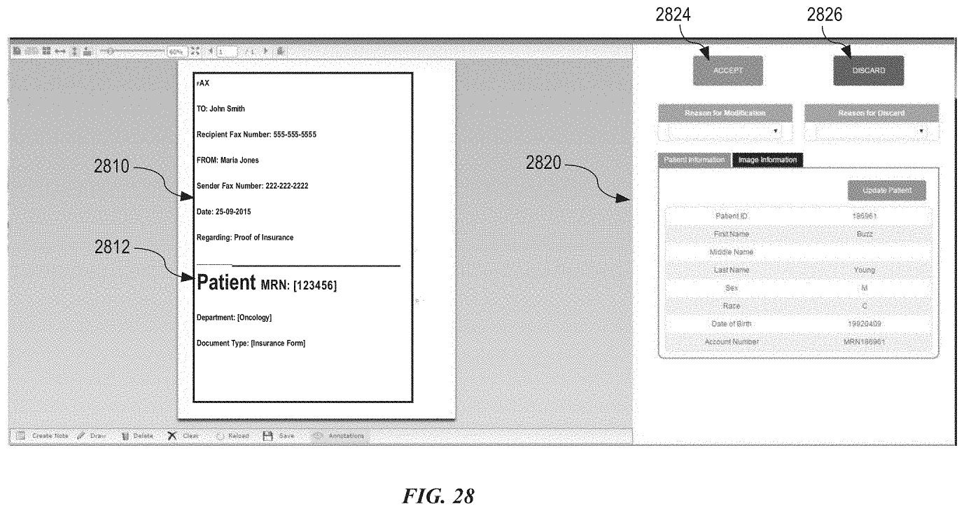

FIG. 28 depicts an example GUI that allows a user to validate an image if a patient record for the image was verified. The GUI depicted in FIG. 28 is one of many examples of the GUI configured to allow the user to review an image, metadata and determine if the image is to be accepted or discarded when a patient record for the image has been verified.

In an embodiment, a GUI is displayed for a user to assist the user in determining whether an image for which a corresponding patient record has been verified is to be accepted or discarded. The GUI may include a first display portion 2810, in which a content of the particular image is displayed, and a second display portion 2820, in which a corresponding patient record is displayed. By examining the displayed information, a user may determine whether the image is to be accepted or discarded even if the corresponding patient record has been verified. For example, a user may determine that the image is to be discarded because, even though a corresponding patient record for the image has been found, the quality of the image is unacceptable.

In the depicted example, first display portion 2610 shows a fax. The fax includes various types of information, including a MRN number 123456 of a patient. Some other information is also included in the fax; however, that information may be difficult to read or discern.

The depicted MRN number 123456 indicates that the fax is to be associated with a patient record of the patient whose MRN number is 123456. The MRN number 123456 may be used to generate a search query, and the search query containing the number 123456 may be used to retrieve a patient record that is associated with the MRN number 123456. The patient record corresponding to the MRN 123456 may be displayed in second display portion 2820.

However, even if a corresponding patient record is found for the received fax, a user may be concerned with the quality of the fax. For example, if some information is illegible, then associating that fax with the corresponding patient record may be undesirable. A user may use interactive button displayed in second display portion 2620 to determine whether the received fax is to be associated with the corresponding patient record or discarded.

Second display portion 2620 may also display an interactive accept button 2824 and an interactive discard button 2826.

Interactive accept button 2824 may be used to accept the fax displayed in first display portion 2610 even if a corresponding patient record has been verified. For example, a user may inspect the fax displayed in first display portion 2810, and determine that the fax is to be accepted and associated with the corresponding patient record even though the quality of the fax is insufficient. If the user determines that the fax is to be accepted and associated with the corresponding patient record, then the user may select interactive accept button 2824 to cause for example, associating the fax with the corresponding patient record.

However, if a user determines that the fax displayed in first display portion 2810 is not be associated with the corresponding patient record, then the user may select interactive discard button 2826 to discard the fax. In some implementation, that may cause displaying another GUI to allow the user to double check whether the fax is indeed to be discarded.

The above example may be expanded to any type of digital images and electronic documents. Further, the example may facilitate providing different types of metadata, and the like.

E. Metadata Modification

In an embodiment, a process of integrating an image with patient records includes augmenting or adding content of metadata associated with the image.

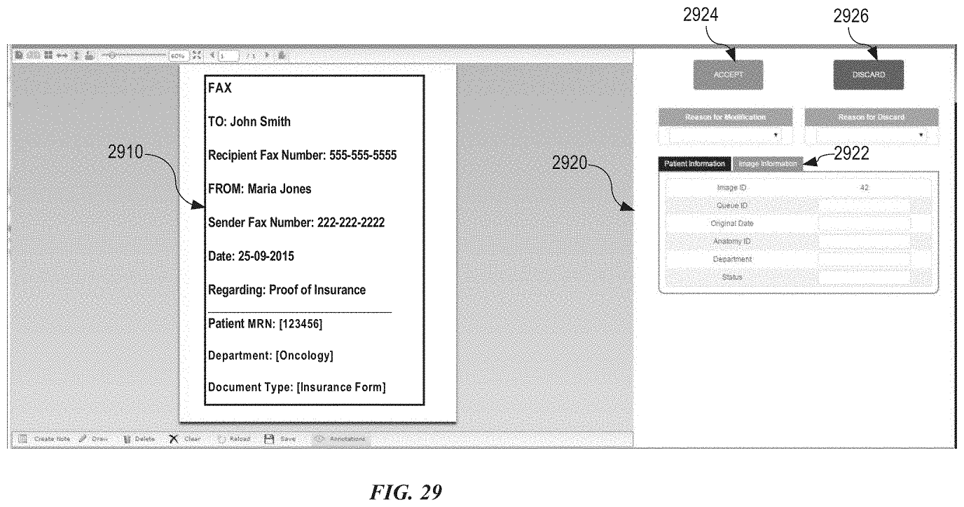

FIG. 29 depicts an example GUI that allows a user to augment metadata associated with the image. The GUI depicted in FIG. 29 is one of many examples of the GUI configured to allow the user to review an image, metadata and augment the metadata associated with the image.

In an embodiment, a GUI is displayed for a user to assist the user in augmenting metadata associated with the image. The GUI may include a first display portion 2910, in which a content of the particular image is displayed, and a second display portion 2920, in which a sub-GUI for augmenting the metadata associated with the image is displayed. Second display portion 2920 may also include interactive buttons, such as an interactive-accept button 2924 and an interactive-discard button 2926.

In the depicted example, first display portion 2910 shows a fax. The fax includes various types of information, including a MRN number 123456 of a patient. The MRN number 123456 may be used to generate a search query, and the search query containing the number 123456 may be used to retrieve a patient record that is associated with the MRN number 123456. The search results may be displayed in second display portion 2820.

However, if a user selects an interactive tab 2922, an additional GUI may be displayed to assist the user in augmenting metadata associated with the image. Using the additional GUI, the user may provide additional information that further describes the received image. In the example depicted in FIG. 29, the additional GUI is labelled as a “image information” GUI. The image information GUI includes several text boxes to which the user may insert for example, an image identifier, a queue identifier, an original date when the image was acquired, an identifier of the anatomy part depicted in the image, an identifier of the department, an identifier of the status, and the like. If some of that information has been already included in the metadata associated with the image, then the user may overwrite it with the new information. If some of that information has not been already included in the metadata associated with the image, then by providing that information to the GUI 2922, the user may augment the metadata associated with the image.

Interactive accept button 2624 may be used to accept the fax displayed in first display portion 2910. For example, a user may inspect the fax displayed in first display portion 2910, augment the metadata associated with the image, and determine that the fax is to be accepted. If the user determines that the fax is to be accepted, then the user may select interactive accept button 2924 to cause associating the image with the corresponding patient record.

However, if a user determines that the fax displayed in first display portion 2910 is not be further processed, then the user may select interactive discard button 2926 to discard the fax.

### XVIII. OVERVIEW OF A METADATA ASSIGNMENT PROCESS

In an embodiment, an approach for assigning metadata to digital images and electronic documents is presented. The approach has especial applicability in service provider systems that process massive amounts of documents. The approach may have a particular applicability in healthcare provider systems that process vast amounts of documents, such as patient identification documents, results of laboratory tests, X-rays, faxes and notes from physicians and nurses, disclosures and authorizations obtained from patients, and the like. These documents may be represented in a digital form as text files, image files or combinations of text and image files. The digital files may include electronic documents and digital images.

In an embodiment, an approach for assigning metadata to digital images and electronic documents allows associating digital images with metadata that in some way describes the digital images. For example, the approach allows associating a digital image includes results of a laboratory test performed for a patient with metadata that provides some details about the image.

Contents included in metadata to be associated with a digital image may depend on a type of the image. For example, if a received image includes results of a laboratory test performed for a patient, then metadata to be associated with the image may include an identifier of the patient, an identifier of an employee who performed the test, an identifier of a department at which the test was performed, a type of the document, an identifier of the document, and the like.

Metadata to be associated with received digital images may be used to associate the received images with patient records maintained by EMR systems. For example, if a received image depicts a photograph of a wound on a patient's hand, then metadata associated with the image may provide identification of the patient, and other information that can be helpful in identifying a patient record of the patient and associating the image with the identified patient record. Once the image and the metadata are associated with the patient record and ported to an EMR system, the image and the associated information are easily accessible to healthcare providers.

In an embodiment, metadata to be associated with a received image is provided via a GUI. The GUI allows entering the metadata to a computer system, associating the metadata with the image, and making the image and the metadata accessible to an EMR system. The GUI may also be configured to verify and validate the image, the metadata and a suggested association between the image and the metadata. Furthermore, the GUI may be configured to modify the metadata and the associations between the images and the metadata.

In an embodiment, an approach allows providing contents of metadata from various devices and computer configurations. For example, metadata may be captured using stationary image capturing devices such as scanners and desktop computers, and portable devices such as cameras, smart phones, tablets, and the like.

Metadata may be provided to a system in many forms. For example, metadata represented as a barcode included in a hardcopy of the document may be scanned using a scanner, and an electronic representation of the barcode may be transmitted from the scanner to an application server implementing the approach. According to another example, an electronic representation of metadata may be transmitted from the scanner to a file transfer protocol server, or sent to a computer server as an email or an email attachment. Furthermore, an electronic representation of metadata may be transmitted directly to a data folder maintained by a computer server.

In an embodiment, metadata may be displayed for a user in a variety of devices. For example, metadata may be displayed on stationary computer devices and on portable devices. The metadata may be shown in a GUI generated and displayed on any type of computer device, and may be modified by the user via the GUI.

A request to associate an image with metadata may be generated automatically or manually by a user. Examples of situations when the request to associate an image with metadata is generated include the following situations: the image and the metadata comprise an indication of a same patient; the image and the metadata are stored in a same file directory; a first file containing the image and a second file containing the metadata have a same file name but different file extensions; the image and the metadata are received in a same electronic communication; the image and the metadata are retrieved from a same storage location; first information indicating that the image and the metadata belong to a same patient is received; second information indicating that the image and the metadata belong to a same patient record is received; and a request to associate the image and the metadata is received.

A GUI may be configured to allow the user to verify and validate received digital images and associated images. For example, a user may examine a received image and retrieved metadata to determine whether the metadata corresponds to the image and whether the metadata is to be associated with the image. This may be accomplished by examining identifiers depicted in the image with identifiers included in the metadata and determining whether they match. For example, if a received image includes a fax that has a cover page indicating that the fax pertains to a particular patient, but the metadata received for the received image indicates that the fax pertains to a patient other than the particular patient, then a user may invalidate the metadata, modify the metadata, or simply reject the metadata.

A GUI may also be configured verify and validate an association between a received image and retrieved metadata. Both, the image may and the metadata may be displayed for the user and allow the user to accept the association, reject the association, or modify the association.

In an embodiment, a GUI allows a user to accept an association between a received image and metadata, and cause sending the image, the metadata and the association to an EMR system.

### XIX. WORKFLOW OF A METADATA ASSIGNMENT PROCESS

A metadata assignment process may be implemented in various service provider applications, including healthcare-related applications, and the like. The process may be performed by executing one or more management applications configured to create, retrieve and assign metadata to the received electronic documents and digital images. The management applications may be executed on one or more application servers hosted at healthcare facilities or available to healthcare service providers. Alternatively, the management applications may be implemented on end-point devices, such as MFPs, or on client devices. An application server may be a computer server configured as a virtual server on a cloud system, a physical server maintained by a service provider, or any other server accessible to a service provider.

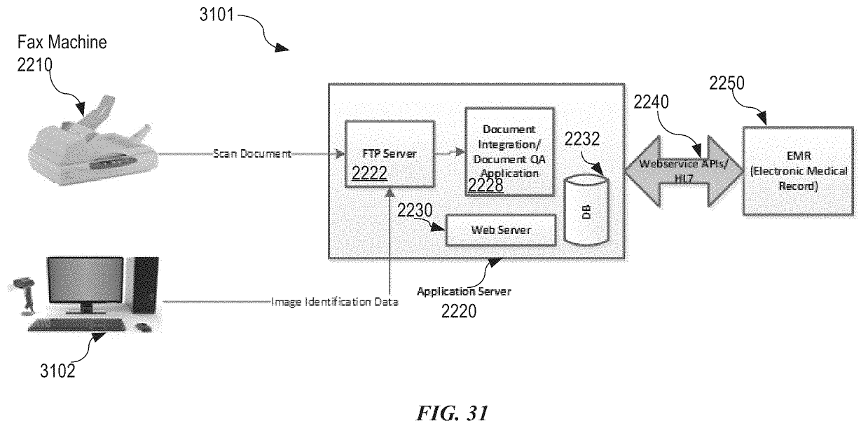

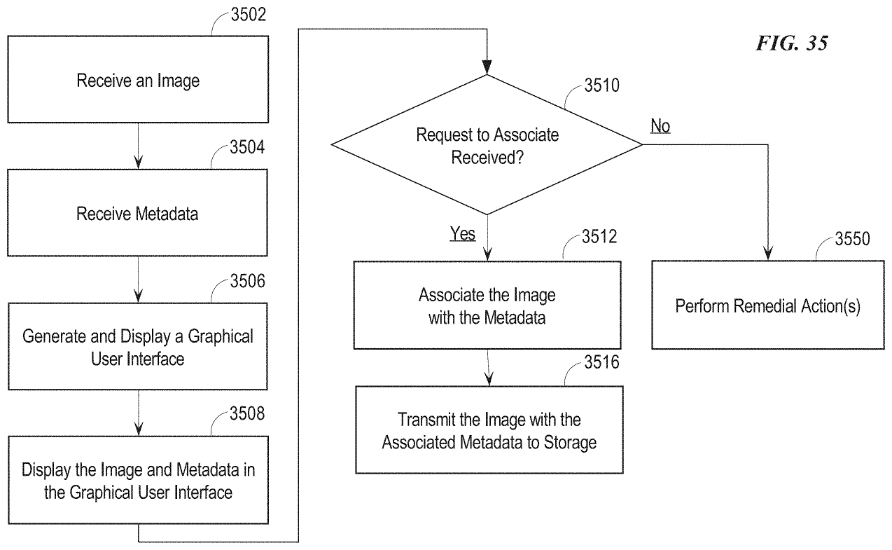

FIG. 35 depicts an example workflow for a metadata assignment process. In step 3502, a management application receives an image acquired by one or more devices. The image may be received from any source, and may be transmitted to a management application using various communications protocols and media. Examples are provided in FIG. 31-34.

In step 3504, a management application receives metadata for an image. At this point, it is not unknown whether the metadata corresponds to the image received in step 3502. The metadata may be received from any location and an address of the location may be indicated using various methods. For example, a location of the metadata may be indicated using a URL, a name of a data folder maintained on a computer server, a name of a data file stored on a computer server, a name of a drive defined in a storage system or a cloud system, and the like.

In step 3506, a graphical user interface is generated and displayed on a computer device for a user. The graphical user interface may have one or more display portions. For example, the graphical user interface may include a first portion for displaying an image, a second portion for displaying metadata, and a third portion for displaying one or more first interactive objects and elements for processing the image and the metadata.

In step 3508, a managing application displays contents of the image and contents of metadata in a GUI. For example, contents of the image may be displayed in a first portion of the GUI, and contents of the metadata may be displayed in a second portion of the GUI.

In addition, one or more interactive buttons may be displayed in the GUI. For example, one of the interactive buttons may be labelled as “Accept” to indicate that a received image is to be associated with the received metadata. Another interactive button may be labeled as “Reject” to indicate that a received image is not to be associated with the received metadata. Other interactive buttons, such a textbox for providing “Reasons for Modification,” or a textbox for providing “Reasons for Discarding,” and the like, may also be displayed.

In step 3510, a management application determines whether a received image is to be associated with received metadata. This may be performed as a verification step, in which the received image and the received metadata are inspected and/or processed.

Processing of an image and metadata may include verifying whether the image is to be associated with the metadata. Verification may include determining whether the image and the metadata are to be associated with the same patient record, with the same patient, and the like.

Determining whether a received image is to be associated with received metadata may be performed automatically or manually. An automatic approach would include, for example, a management application inspecting the content of the image and the content of the metadata, and determining whether both the image and the metadata include some indicia specifying that the image is to be associated with the metadata. If such indicia are present, then the management application may generate a request or recommendations for associating the image with the metadata.

Determining whether a received image is to be associated with received metadata may also be performed manually by a user. For example, a user may examine a display of the content of the image and the content of the metadata, and determine whether the image and the metadata are to be associated with each other. If the user determines that the two are to be associated, then a user may select one of the interactive buttons provided in a GUI to generate a request to associate the image with the metadata.

An automatic and/or manual determination of whether a received image is to be associated with received metadata may be performed based on various factors. For example, a management application or a user may determine that a received image is to be associated with received metadata if the image and the metadata comprise an indication of a same patient, if the image and the metadata are stored in a same file directory, or if a first file containing the image and a second file containing the metadata have a same file name but different file extensions. A management application or a user may determine that a received image is to be associated with received metadata if for example, the image and the metadata are received in a same electronic communication, or the image and the metadata are retrieved from a same storage location, or first information indicating that the image and the metadata belong to a same patient is received, or second information indicating that the image and the metadata belong to a same patient record is received. The determination may also be based on receiving a request to associate the image and the metadata from other users, other computer applications, and the like. For example, when system receives an image, a unique token may be generated, and sent along with metadata to be used by the association process.

In 3510, it is determined whether a request to associate a received image with a received metadata is received. The request may be received from a user or from a management application. For example, upon inspecting contents of the received image and contents of the received metadata, a user may determine that the image and the metadata are to be associated, and thus generate a request to associate the received image and the received metadata. This may also be performed automatically, as described above.

If a request to associate a received image with received metadata is received, then in step 3512, the image is associated with the metadata, and the association of the image and the corresponding metadata is in step 3516 transmitted to for example, an EMR system. An association between the image and the metadata may be created by generating additional metadata indicating the association, and adding the additional metadata to the image file or to the metadata file. The association may also be saved in a separate file and an URL to the file may be included in the image file or the metadata file.

However, if a request to associate a received image with received metadata is not received or an explicit request not to associate the received image with the received metadata is received, then in step 3550 one or more remedial actions are performed. For example, a user may be prompted to provide reasons for disallowing an association between the received image and the received metadata. A user may also be prompted to provide one or more additional interactive buttons or textboxes for providing additional information about the image and/or the metadata.

A user may also be prompted to provide a first user input specifying a location for storing a received image in association with received metadata. Alternatively, a user may be provided with the information about the location at which the received image is to be stored in association with the metadata. For example, a user may be prompted to provide a name of the server, such as a name of the EMR server, or a name of the directory on the server to which the association between the image and the corresponding metadata is to be transmitted. Alternatively, a user may be provided with a URL, which the user may use to transmit the association between the image and the corresponding metadata.

### XX. EXAMPLE METADATA ASSIGNMENT PROCESS

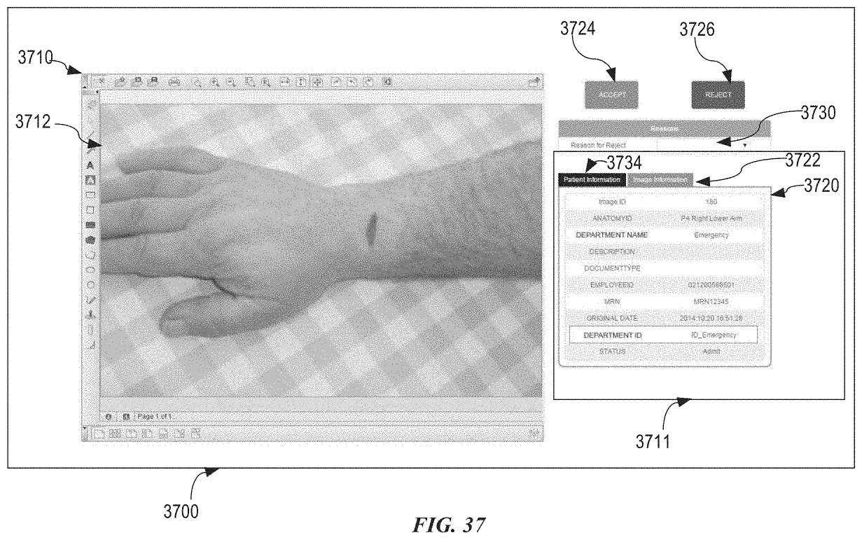

A process of assigning metadata to an image implemented at a healthcare provider facility may be illustrated using an example depicted in FIG. 37.

FIG. 37 depicts an example interface 3700 for interactively assigning metadata to images. Interface 3700 may be implemented as a GUI 370, as depicted in FIG. 37. GUI 3700 may include a first portion 3710 and a second portion 3711. First portion 3710 may be used to display a received image 3712. Second portion 3711 may be used to display a header 3722 explaining the contents displayed in second portion 3711 and received metadata 3720.

Furthermore, GUI 3700 may include one or more interactive buttons. For example, GUI 3700 may include an “Accept” button 3724, a “Reject” button 3726, and the like.

Moreover, GUI 3700 may include one or more textbox for providing alphanumerical information to a management application. For example, GUI 3700 may include a “Reasons for Reject” textbox 3730 for providing an explanation as to why a user determined that a received image is not to be associated with received metadata.

Upon receiving an image, a management application may cause displaying GUI 3700. Contents of the received image 3712 may be displayed in first portion 3710 of GUI 3700.

Upon receiving metadata, a management application may cause displaying contents of metadata 3720 in second portion 3711 of GUI 370. Additional buttons displayed in GUI 3700 may include “Accept” button 3724 to cause an association between a received image and received metadata, and “Reject” button 3726 to reject associating the received image with the received metadata.

Furthermore, “Reason to Reject” textbox 3730 may be displayed in GUI 3700 to allow a user to provide additional information about reasons for not associating a received image with received metadata.

A received image may be a fax communication, a file attachment, a scan image, and the like. The received image may include metadata or an indication of a patient identification. The identification may include a patient record, a social security number of the patient for whom the image was received, and the like. The identification may be determined by processing the received image using an OCR reader, a bar code reader, a QR code reader, and the like. For example, a received image may include an imprinted bar code. The bar code may be scanned using a bar code, converted to an alphanumerical string, and the alphanumerical string may be displayed in first portion 3710 of GUI 3700.

Received metadata may be stored in a particular data file stored on a server, in a particular file directory, or at a particular URL. Received metadata may include a patient record, a social security number of the patient for whom the image was received, and the like. The metadata may be determined by processing encoded information using an OCR reader, a QR code reader, and the like. For example, a laboratory technician who take a photograph of hand of a patient may generate a bar code that represents information about the patient whose hand is depicted in the photograph, and store the bar code at a particular URL.

Receiving metadata for an image may include providing a name of the server, a file directory on the server, or an URL of a location on a server at which the metadata is stored, and downloading the metadata from the specified location.

A metadata assignment process may be implemented as an interactive process in which corresponding contents are displayed for a user in GUI 3700, and the user may inspect the contents and perform one or more actions on the contents. For example, a received image and received metadata may be verified or inspected by an authorized person or a manager, and if the image and the metadata are to be associated with each other, a user may select “Accept” interactive button 3724 to cause a management application to associate the image with the corresponding metadata. For example, if image information 3722 displayed in second portion 3711 of GUI 3700 indicates that a patient MRN that corresponds to a patient MRN associated with patient information 3734 displayed in second portion 3711 of GUI 3700 (or in first portion 3710 of GUI 3700), then a user may select “Accept” interactive button 3724 to cause a management application to associate the received image with the received metadata because the MRN numbers match.

If an association between a received image and received metadata is requested or if the association is approved, then an indication of the association, the received image and/or the associated metadata may be transmitted to an EMR system of a healthcare service provider.

However, if a user determines that a received image is not to be associated with received metadata, then a user may select “Reject” interactive button 3726 to cause a management application not to associate the received image with the received metadata. For example, if image information 3722 displayed in second portion 3711 of GUI 3700 indicates that a patient MRN that is different from a patient MRN associated with patient information 3734 displayed in second portion 3711 of GUI 3700 (or in first portion 3710 of GUI 3700), then a user may select “Reject” interactive button 3726 to cause a management application not to associate the received image with the received metadata because the MRN numbers do not match.

In an embodiment, if an association between a received image and received metadata is rejected, then a user may be prompted to provide reasons for rejecting the association. For example, a user may be prompted to provide the reasons in “Reasons for Reject” textbox 3730. In textbox 3730, the user may type in an explanation for rejecting the association, provide suggestions for verifying the image and/or the metadata, or refer the image for further review or evaluation.

### XXI. ARRANGEMENTS FOR ASSIGNING METADATA TO IMAGES

A process of assigning metadata to an image may be implemented using a variety of hardware-software-based arrangements. Examples of the arrangements may include arrangements in which the digital images are received from MFP devices, cameras, or any other image configured to transmit digital images, and in which metadata is captured using desktop applications, tablets, portable devices, cameras, and the like.

The arrangements may further be divided into several groups. One group may include the arrangements in which received images and received metadata are transmitted to an FTP-based server using for example, an FTP-based data transfer. Another group may include the arrangements in which received images and received metadata are transmitted using an email transfer protocol to an email server. Other groups may include the arrangements in which received images and received metadata are transmitted using the FTP protocol to a file directory stored on a server. Other arrangements may also be implemented.

A. Example Arrangements For Providing Metadata for Images Using a Desktop Computer

In an embodiment, a process of assigning metadata to images is implemented using arrangements in which the metadata is captured or otherwise provided to a management application using a desktop computer.

FIG. 31 depicts a block diagram that depicts an arrangement 3101 for capturing metadata for images using a desktop computer. In the depicted example, arrangement 3101 includes an image providing device, such as a MFP device 2210, and a desktop computer 3102. Digital images are received from an MFP device 2210 and transmitted to a file server, such as for example, an FTP server 2222. In arrangement 3101, MFP device 2210 is communicatively coupled with an application server 2220, which is communicatively coupled with one or more EMR systems 2250.

In arrangement 3101, MFP device 2210 is any type of a MFP configured to receive and process any type of electronic data. MFP device 2210 is described in detail in FIG. 22A.

Application server 2220 may include one or more components that are configured to receive and process digital image data and metadata. Application server 2220 may include for example, an FTP server 2222, and a document integration and processing component 2228. Application server 2220 may also include one or more web servers 2230 and/or may communicate with one or more external web servers (not depicted in FIG. 22A). Furthermore, application server 2220 may include one or more data storages, such as for example, a database 2232. Application server 2220 is described in detail in FIG. 22A.

Desktop computer 3102 is any type of a computing device configured to receive and transmit digital data. Desktop computer 3102 may be a personal computer, a lap top, or any other computing device.

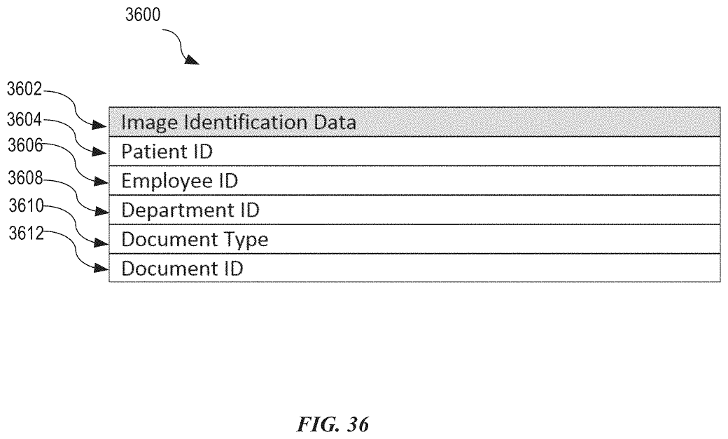

In an embodiment, desktop computer 3102 is configured to execute a computer application allowing capturing metadata to be associated with an image received from MFP device 2210. This may be accomplished by launching a computer application on desktop computer 3102 that in turn generates a GUI that allows a user to enter metadata for a corresponding image. Examples of metadata items are described in FIG. 36.

Entering metadata for a corresponding image may be accomplished by allowing a user to type in metadata items using a GUI displayed on a display device. For example, a user may type in information about a patient name, a patient medical record, and the like. The metadata items may be saved in a metadata file stored on FTP server 2222.

Metadata for a corresponding image may also be provided using scanning devices. For example, a user of desktop computer 3102 may use a scanner to scan a bar code associated with an image, and transmit the scanned code to FTP server 2222. A user may also use a bar code reader to scan and decode a bar code associated with an image, and transmit the decoded bar code to FTP server 2222. A user may also use a QR code reader to scan and decode a QR code associated with an image, and transmit the decoded bar code to FTP server 2222.

Metadata for a corresponding image may also use a camera attached to desktop computer 3102 to capture an image, and then use an OCR application to translate the captured image into an alphanumerical text, display the alphanumerical text on a display device and allow a user to determine metadata for the captured image. For example, if a captured image is a photograph of an injured hand, as depicted in FIG. 37, a user may use an OCR application to translate a portion of the captured image into a text including a patient record of the patient whose hand is depicted in the captured image, and manually enter the patient record into a GUI used to provide the metadata for the captured image. Once the metadata is provided, the metadata may be transmitted to FTP server 2222.

Document integration and processing component 2228 is configured to determine whether a received digital image may be association with received metadata. This may be accomplished by executing a management application described in detail in FIG. 35.

Furthermore, component 2228 may be configured to assist a user of application server 2220 in reviewing received digital images, received metadata and possible associations between the received digital images and the received metadata. Details of the process for associating an image with metadata is described in FIG. 35.

B. Example Arrangements For Providing Metadata For Images Using a Portable Device

In an embodiment, a process of assigning metadata to images is implemented using arrangements in which the metadata is captured or otherwise provided to a management application using one or more portable devices, such as tablets, smart phones, and the like.

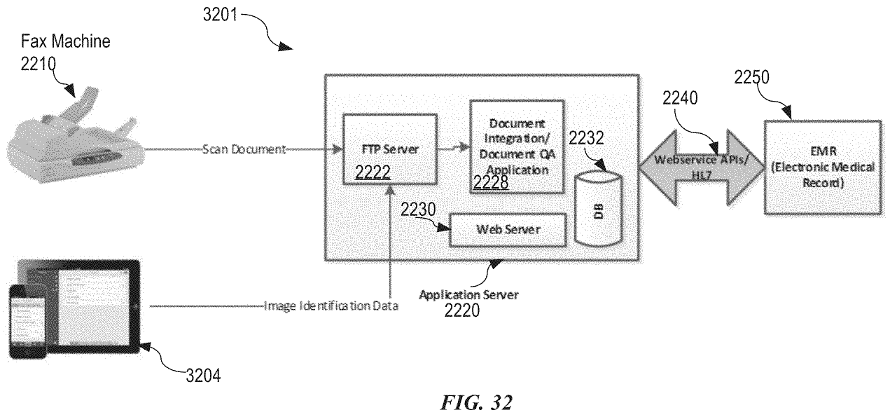

FIG. 32 depicts a block diagram that depicts an arrangement 3201 for capturing metadata for images using a portable device. In the depicted example, arrangement 3201 includes an image providing device, such as a MFP device 2210, and one or more portable devices 3204. Digital images are received from an MFP device 2210 and transmitted to a file server, such as for example, an FTP server 2222. In arrangement 3201, MFP device 2210 is communicatively coupled with an application server 2220, which is communicatively coupled with one or more EMR systems 2250.

In arrangement 3201, MFP device 2210 is any type of a MFP configured to receive and process any type of electronic data. MFP device 2210 is described in detail in FIG. 22A.

Application server 2220 may include one or more components that are configured to receive and process digital image data and metadata, and that are described in detail in FIG. 22A.

One or more portable devices 3204 include any type of portable computing devices configured to receive and transmit digital data. For example, one or more portable devices 3204 may include a laptop, a tablet, a smart phone, and the like.

In an embodiment, a portable device 3204 is configured to execute a computer application allowing capturing metadata to be associated with an image received from MFP device 2210. This may be accomplished by launching a computer application on portable device 3204 that in turn generates a GUI that allows a user to enter metadata for a corresponding image. Examples of metadata items are described in FIG. 36.

Entering metadata for a corresponding image may be accomplished by allowing a user to type in metadata items using a GUI displayed on a display device. For example, a user may type in information about a patient name, a patient medical record, and the like. The metadata items may be saved in a metadata file stored on FTP server 2222.

Metadata for a corresponding image may also be provided using scanning devices. Examples of providing metadata via a scanner are described in FIG. 31.

Metadata for a corresponding image may also use a camera attached to desktop computer 3102 to capture an image, and then use an OCR application to translate the captured image into an alphanumerical text, display the alphanumerical text on a display device and allow a user to determine metadata for the captured image. Examples of providing metadata via a camera are described in FIG. 31.

Document integration and processing component 2228 is configured to determine whether a received digital image may be association with received metadata. This may be accomplished by executing a management application described in detail in FIG. 35.

Furthermore, component 2228 may be configured to assist a user of application server 2220 in reviewing received digital images, received metadata and possible associations between the received digital images and the received metadata. Details of the process for associating an image with metadata is described in FIG. 35.

C. Example Arrangements for Communicating Images and Metadata as

Attachments

In an embodiment, images and corresponding metadata are transmitted using an electronic mail server to an email client module implemented in an application server. In the corresponding arrangements, the images and the metadata are transmitted using an email transfer protocol to the email server as attachments.

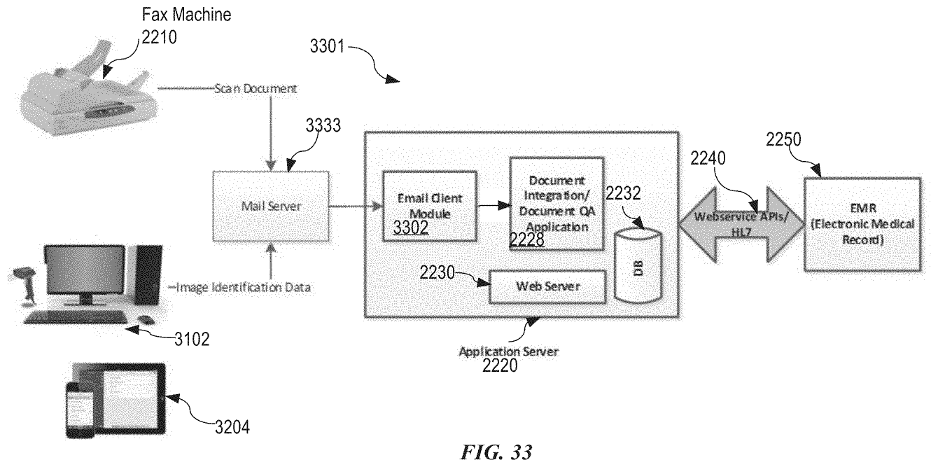

FIG. 33 depicts a block diagram that depicts an arrangement 3301 for transmitting images and metadata as electronic mail attachments. In the depicted example, arrangement 3301 includes an image providing device, such as a MFP device 2210, a desktop computer 3102, and/or one or more portable devices 3204. Digital images are received from an MFP device 2210 and transmitted to an email server 3333. In arrangement 3301, MFP device 2210 is communicatively coupled with an application server 2220, which is communicatively coupled with one or more EMR systems 2250.

In arrangement 3301, MFP device 2210 is any type of a MFP configured to receive and process any type of electronic data. MFP device 2210 is described in detail in FIG. 22A.

Application server 2220 may include one or more components that are configured to receive and process digital image data and metadata, and that are described in detail in FIG. 22A.

Desktop computer 3102 is any type of a computing device configured to receive and transmit digital data. Desktop computer 3102 is described in detail in FIG. 31.

One or more portable devices 3204 include any type of portable computing devices configured to receive and transmit digital data. One or more portable devices 3204 are described in detail in FIG. 32.

In an embodiment, desktop computer 3102 and a portable device 3204 are configured to execute a computer application allowing capturing metadata to be associated with an image received from MFP device 2210. The process of capturing metadata is described in FIGS. 31 and 32. Examples of metadata items are described in FIG. 36.

Metadata may be transmitted from desktop computer 3102 and/or portable devices 3204 as email attachments to email server 3333. Upon receiving the email with the attachment containing the metadata, email server 3333 may forward the email and the attachment to email client module 3302.

An image captured by MFP device 2210 may also be transmitted from MFP device 2210 as email attachments to email server 3333. Upon receiving the email with the attachment containing the image, email server 3333 may forward the email and the attachment to email client module 3302.

Document integration and processing component 2228 is configured to determine whether a received digital image may be association with received metadata. For example, upon receiving an email containing an attachment with an image and an email containing an attachment with metadata, document integration and processing component 2228 may be used to determine whether the received image is to be associated with the received metadata. This may be accomplished by executing a management application described in detail in FIG. 35.

Furthermore, component 2228 may be configured to assist a user of application server 2220 in reviewing received digital images, received metadata and possible associations between the received digital images and the received metadata. Details of the process for associating an image with metadata is described in FIG. 35.

D. Example Arrangements for Transmitting Images and Metadata to a Data Folder

In an embodiment, images and corresponding metadata are transmitted to an electronic data file implemented in an application server. In the corresponding arrangements, the images and the metadata are transmitted using an email transfer protocol to an electronic data file implemented as a file directory, a file folder, and the like.

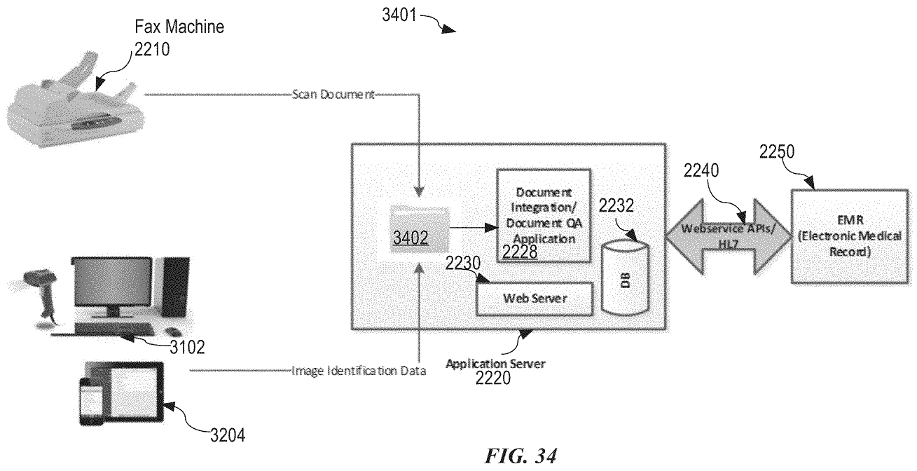

FIG. 34 depicts a block diagram that depicts an arrangement 3401 for transmitting images and metadata to an electronic data folder. In the depicted example, arrangement 3401 includes an image providing device, such as a MFP device 2210, a desktop computer 3102, and/or one or more portable devices 3204. Digital images are received from an MFP device 2210 and transmitted to an electronic data folder 3402. In arrangement 3401, MFP device 2210 is communicatively coupled with an application server 2220, which is communicatively coupled with one or more EMR systems 2250.

In arrangement 3401, MFP device 2210 is any type of a MFP configured to receive and process any type of electronic data. MFP device 2210 is described in detail in FIG. 22A.

Application server 2220 may include one or more components that are configured to receive and process digital image data and metadata, and that are described in detail in FIG. 22A.

Desktop computer 3102 is any type of a computing device configured to receive and transmit digital data. Desktop computer 3102 is described in detail in FIG. 31.

One or more portable devices 3204 include any type of portable computing devices configured to receive and transmit digital data. One or more portable devices 3204 are described in detail in FIG. 32.

In an embodiment, desktop computer 3102 and/or a portable device 3204 are configured to execute a computer application allowing capturing metadata to be associated with an image received from MFP device 2210. The process of capturing metadata is described in FIGS. 31 and 32. Examples of metadata items are described in FIG. 36.

Metadata may be transmitted from desktop computer 3102 and/or portable devices 3204 to electronic data folder 3402. Electronic data folder 3402 may be a shared data folder 3402 implemented in one or more servers, in a cloud system, and the like. Transfer of an image from MFP device 2210 to shared folder 3402 may be performed automatically via a secure or an unsecure connection established between MFP 2201 and application server 2220.

Upon receiving an indication that an image and corresponding metadata have been transmitted to shared folder 3402, document integration and processing component 2228 is invoked to facilitate association between the image and the metadata.

Document integration and processing component 2228 is configured to determine whether a received digital image may be association with received metadata. For example, upon receiving an email containing an attachment with an image and an email containing an attachment with metadata, document integration and processing component 2228 may be used to determine whether the received image is to be associated with the received metadata. This may be accomplished by executing a management application described in detail in FIG. 35.

Furthermore, component 2228 may be configured to assist a user of application server 2220 in reviewing received digital images, received metadata and possible associations between the received digital images and the received metadata. Details of the process for associating an image with metadata is described in FIG. 35.

### XXII. EXAMPLE METADATA

Metadata to be associated with an image may include any type of information that in some way describes a received image, a person for whom the image was taken or who is depicted in the image, or any other characteristics of the image or the person that may be useful in identifying the image. Metadata may be represented using any known data structure, such as a data table, a data list, a data containers linked using pointers, and the like.

FIG. 36 depicts an example data structure 3600 used to store metadata information. Example data structure 3600 is provided herein to illustrate examples of data that may be used to characterize a digital image. Example data structure 3600 may be implemented as a data table, a list, and the like.

In an embodiment, example, data structure 3600 includes a metadata header 3602. Metadata header 3602 may include an alphanumerical string that provides a description of the data structure. In the depicted example, metadata header 3602 indicates that the metadata comprises “Image Identification Data.”

In health-care related applications, example data structure 3600 may also include information about a patient for whom a corresponding image was captured or who is depicted in the corresponding image. In the depicted example, data structure 3600 includes a patient identifier 3604 of a patient for whom a corresponding image was captured, an employee ID 3606 of an employee who captured the corresponding image, a department ID 3608 of a department that requested capturing the corresponding image or that requested laboratory tests depicted in the corresponding image, a document type 3610 of a document that comprises the metadata (or the corresponding image), a document ID 3612 of a document that comprises the metadata (or the corresponding image), and the like. In other implementations, other types of data may be stored in metadata structures.

### XXIII. EXAMPLE INTERFACE FOR INTERACTIVELY ASSIGNING METADATA TO IMAGES

A user interface configured to allow assigning metadata to images may be implemented in a variety of way. For example, the interface may be implemented as a GUI, as a questionnaire, and the like.

FIG. 37 depicts an example interface 3700 for interactively assigning metadata to images. As described above, GUI 3700 may include a first portion 3710 and a second portion 3711. First portion 3710 may be used to display a received image 3712. Second portion 3711 may be used to display a header 3722 explaining the contents displayed in second portion 3711 and received metadata 3720. GUI 3700 may also include one or more interactive buttons. For example, GUI 3700 may include an “Accept” button 3724, a “Reject” button 3726, and the like. Furthermore, GUI 3700 may include one or more textbox for providing alphanumerical information to a management application. For example, GUI 3700 may include a “Reasons for Reject” textbox 3730 for providing an explanation as to why a user determined that a received image is not to be associated with received metadata.

In an embodiment, contents of a received image 3712 is displayed in first portion 3710 of GUI 3700, and contents of metadata 3720 is displayed in second portion 3711 of GUI 370.

A received image may be a fax communication, a file attachment, a scan image, and the like. Received metadata may include a patient record, a social security number of the patient for whom the image was received, and the like. The metadata may be determined by processing encoded information using an optical character recognition (OCR) reader, a quick response (QR) code reader, and the like. For example, a laboratory technician who take a photograph of hand of a patient may generate a bar code that represents information about the patient whose hand is depicted in the photograph, and store the bar code at a particular URL.

A metadata assignment process may be implemented as an interactive process in which a received image and received metadata may be verified or inspected by an authorized person or a manager, and if the image and the metadata are to be associated with each other, a user may select “Accept” interactive button 3724 to cause a management application to associate the image with the corresponding metadata. If an association between a received image and received metadata is requested or if the association is approved, then an indication of the association, the received image and/or the associated metadata may be transmitted to an EMR system of a healthcare service provider.

However, if a user determines that a received image is not to be associated with received metadata, then a user may select “Reject” interactive button 3726 to cause a management application not to associate the received image with the received metadata. In an embodiment, if an association between a received image and received metadata is rejected, then a user may be prompted to provide reasons for rejecting the association. For example, a user may be prompted to provide the reasons in “Reasons for Reject” textbox 3730. In textbox 3730, the user may type in an explanation for rejecting the association, provide suggestions for verifying the image and/or the metadata, or refer the image for further review or evaluation.

A process of associating metadata with an image allows efficient processing of a vast amount of images and corresponding data. The approach allows automatically analyzing received images and received images, verifying and validating the received images and metadata, determining whether the received images and the metadata are to be associated, and if so, causing the determines associations.

A process of associating metadata with an image allows an integration of data from different sources with an EMR system. The process of associating the metadata with the image may allow processing the images and porting the determined associations between the images and the corresponding metadata to the EMR systems.

1.-20. (canceled)

21. A document integration processing system comprising:
one or more memory units;
one or more processors;
wherein the document integration processing system is configured to:
receive an electronic document, including one or more digital images, from a device;
translate the one or more digital images into a machine-encoded-text;
automatically extract one or more data items, including at least patient identification data, from the machine-encoded-text;
automatically associate the electronic document with a patient information record, of a plurality of patient information records, selected based on the at least patient identification data extracted from the machine-encoded text;
transmit the electronic document associated with the patient information record to one or more computer systems.

22. The document integration processing system of claim 21, further configured to:
automatically classify the electronic document into a type of document.

23. The document integration processing system of claim 22, further configured to:
automatically associate the one or more data items, including the at least patient identification data, with the electronic document;
wherein associating the one or more data items with the electronic document is performed based on and according to the type of document.

24. The document integration processing system of claim 21, wherein the electronic document includes at least one of: medical laboratory test results, medical receipts, medical invoices, or billing documents.

25. The document integration processing system of claim 21, further configured to:
receive the electronic document from the device of a plurality of different devices.

26. The document integration processing system of claim 25, wherein the plurality of different devices includes fax machines, MFP devices, scanners, copiers, and file servers.

27. A document integration processing method comprising:
receiving an electronic document, including one or more digital images, from a device;
translating the one or more digital images into a machine-encoded-text;
automatically extracting one or more data items, including at least patient identification data, from the machine-encoded-text;
automatically associating the electronic document with a patient information record, of a plurality of patient information records, selected based on the at least patient identification data extracted from the machine-encoded text;
transmitting the electronic document associated with the patient information record to one or more computer systems.

28. The document integration processing method of claim 27, further comprising:
automatically classifying the electronic document into a type of document.

29. The document integration processing method of claim 28, further comprising:
automatically associating the one or more data items, including the at least patient identification data, with the electronic document;
wherein associating the one or more data items with the electronic document is performed based on and according to the type of document.

30. The document integration processing method of claim 27, wherein the electronic document includes at least one of: medical laboratory test results, medical receipts, medical invoices, or billing documents.

31. The document integration processing method of claim 27, further comprising:
receiving the electronic document from the device of a plurality of different devices.

32. The document integration processing method of claim 31, wherein the plurality of different devices includes fax machines, MFP devices, scanners, copiers, and file servers.

33. One or more non-transitory computer-readable storage media storing one or more instructions which, when executed by one or more processors, cause the one or more processors to perform:
receiving an electronic document, including one or more digital images, from a device;
translating the one or more digital images into a machine-encoded-text;
automatically extracting one or more data items, including at least patient identification data, from the machine-encoded-text;
automatically associating the electronic document with a patient information record, of a plurality of patient information records, selected based on the at least patient identification data extracted from the machine-encoded text;
transmitting the electronic document associated with the patient information record to one or more computer systems.

34. The one or more non-transitory computer-readable storage media of claim 33, storing additional instructions for: automatically classifying the electronic document into a type of document.

35. The one or more non-transitory computer-readable storage media of claim 34, storing additional instructions for:
automatically associating the one or more data items, including the at least patient identification data, with the electronic document;
wherein associating the one or more data items with the electronic document is performed based on and according to the type of document.

36. The one or more non-transitory computer-readable storage media of claim 33, wherein the electronic document includes at least one of: medical laboratory test results, medical receipts, medical invoices, or billing documents.

37. The one or more non-transitory computer-readable storage media of claim 33, storing additional instructions for: receiving the electronic document from the device of a plurality of different devices.

38. The one or more non-transitory computer-readable storage media of claim 37, wherein the plurality of different devices includes fax machines, MFP devices, scanners, copiers, and file servers.
